(19) United States
(12) Patent Application Publication
(10) Pub. No.: US 2019/0127354 A1
(43) Pub. Date: May 2, 2019

(54) 2,4,6,7-TETRASUBSTITUTED QUINOLINE COMPOUNDS AS INHIBITORS OF DNA METHYLTRANSFERASES

(71) Applicant: FUNDACIÓN PARA LA INVESTIGACIÓN MÉDICA APLICADA, Pamplona (ES)

(72) Inventors: Xabier AGUIRRE ENA, Pamplona (ES); Julen OYARZABAL SANTAMARINA, Pamplona (ES); Felipe PRÓSPER CARDOSO, Pamplona (ES); Maria Obdulia RABAL GRACIA, Pamplona (ES); Edurne SAN JOSÉ ENÉRIZ, Pamplona (ES); Juan Antonio SÁNCHEZ ARIAS, Pamplona (ES)

(21) Appl. No.: 16/061,325

(22) PCT Filed: Dec. 13, 2016
(86) PCT No.: PCT/EP2016/080716

§ 371 Date: Jun. 11, 2018

(30) Foreign Application Priority Data
Dec. 14, 2015 (EP) 15382620.1

(51) Int. Cl. C07D 405/14; C07D 401/14; C07D 405/04; A61P 35/00

(52) U.S. Cl. CPC C07D 405/14; A61P 35/00; C07D 405/04; C07D 401/14

(57) ABSTRACT

It relates to the compounds of formula (I), or their pharmaceutically or veterinary acceptable salts, or their stereoisomers or mixtures of stereoisomers, wherein R is a radical selected from the group consisting of formula (A), formula (B), formula (C), formula (D), and formula (E), and R₁, R₂, and R₃ are as defined herein, which are inhibitors of one or more DNMTs selected from the group consisting of DNMT1, DNMT3A and DNMT3B. It also relates to pharmaceutical or veterinary compositions containing them, and to their use in medicine, in particular in the treatment and/or prevention of cancer, fibrosis and/or immunomodulation.

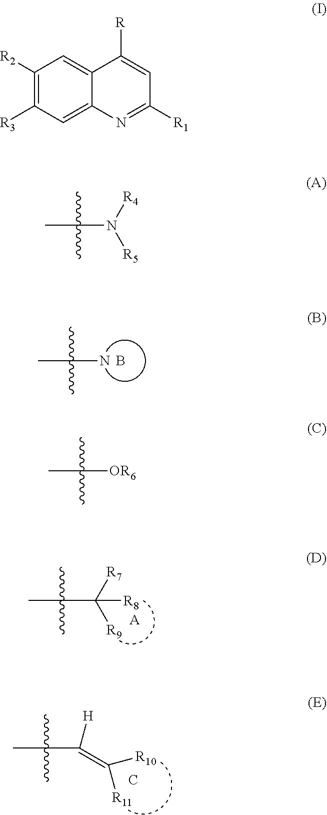

This application claims the benefit of European Patent Application EP15382620.1 filed on Dec. 14, 2015.

The present invention relates to 2,4,6,7-tetrasubstituted quinoline compounds, which are inhibitors of DNA methyltransferases. It also relates to pharmaceutical or veterinary compositions containing them, and to their use in medicine, in particular as anticancer agents, antifibrotic and immunomodulator agents.

### BACKGROUND ART

In recent years, it has been shown that cancer is a genetic and epigenetic disease, where epigenetic and genetic alterations interact reciprocally to drive cancer development. However, unlike genetic mutations, epigenetic changes are reversible, and as such, drugs that restore the epigenetic balance represent exciting potential therapeutic targets for cancer. Epigenetics refers to the heritable changes in gene expression patterns that occur independently of alterations in primary DNA sequence. The main epigenetic mechanisms are DNA methylation and covalent histone modifications, which play important roles in the regulation of transcription.

DNA methylation is an epigenetic modification that modulates gene expression without altering the DNA base sequence and plays a crucial role in cancer by silencing tumor suppressor genes. DNA methyltransferases (DNMTs) are the enzymes that catalyze DNA methylation. DNMT1 encodes the maintenance methyltransferase and DNMT3A and DNMT3B encode de novo methyltransferases.

DNMT1 and DNMT3A/3B are overexpressed in several types of cancer such as breast, gastric, pancreas, prostate, hepatocellular, ovarian, renal, retinoblastoma, glioma or diffuse large B-cell lymphoma. The DNA hypomethylating agents like Zebularine, decitabine and azacytidine inhibits cell proliferation and induce apoptosis in acute lymphoblastic leukemia, acute myeloid leukemia, hepatic carcinoma, lung, breast, gastric or cervical cancer among others (Vilas-Zornoza A. et al., PLoS ONE 2011, 6(2): p. e17012).

Decitabine has been currently approved for myelodysplastic syndrome by the US Food and Drug Administration. On the other hand, DNA methylation plays a key role in the pathogenesis of fibrosis (Neary, R. et al, Fibrogenesis & Tissue Repair 2015, 8:18). Further, DNA methyltransferase inhibition also accelerates the immunomodulation and migration of human mesenchymal stem cells (Lee S. et al., Scientific Reports 2015, 5:8020).

However, many efforts are made to develop new non-nucleoside inhibitors to overcome the limits of these azanucleosides, such as chemical instability and incorporation into DNA for activity.

G9a, also known as EHMT2, is a histone methyltransferase that mono- and dimethylates Lysine 9 of histone H3 (H3K9me1 and H3K9me2, respectively). G9a expression is high in many cancers compared with normal tissue. Cancer transcriptome analysis has revealed high expression in many tumors including hepatocellular, colon, prostate, lung bladder and invasive transitional cell carcinomas and in B cell chronic lymphocytic leukemia (Shankar S R. et al., Epigenetics 2013, 8(1): p. 16-22). Knockdown of G9a in both bladder and lung cancer cell lines caused growth suppression and apoptosis. Studies on prostate cancer further corroborate its role in carcinogenesis, where downregulation of G9a causes centrosome disruption, chromosomal instability, inhibition of cell growth and increased cellular senescence in cancer cells. In aggressive lung cancer, high levels of G9a correlate with poor prognosis with increased cell migration and invasion in vitro and metastasis in vivo. G9a is also overexpressed in pancreatic adenocarcinoma and inhibition of G9a induces cellular senescence in this type of cancer. In Acute Myeloid Leukemia mouse models, loss of G9a significantly delays disease progression and reduces leukemia stem cells frequency.

Interestingly, DNA methyltransferase-1 (DNMT1) physically interacts with G9a to coordinate DNA and histone methylation during cell division (Esteve P O. et al., Genes Dev 2006, 20:3089-3103) promoting transcriptional silencing of target genes (Tachibana M. et al., EMBO J 2008, 27:2681-2690; Auclair G. et al., Genome Research 2015). In this sense, reduction of both DNA and H3K9 methylation levels leads to reactivation of tumor suppressor genes and inhibits cancer cell proliferation (Wozniak R J. et al., Oncogene 2007, 26, 77-90; Sharma S. et al., Epigenetics Chromatin 2012. 5, 3 (2012).

Srimongkolpithak N., et al. (Med. Chem. Commun. 2014, 5, 1821-1828) reports the identification of 2,4-diamino-6,7-dimethoxyquinoline derivatives 41 and 42 as G9a inhibitors.

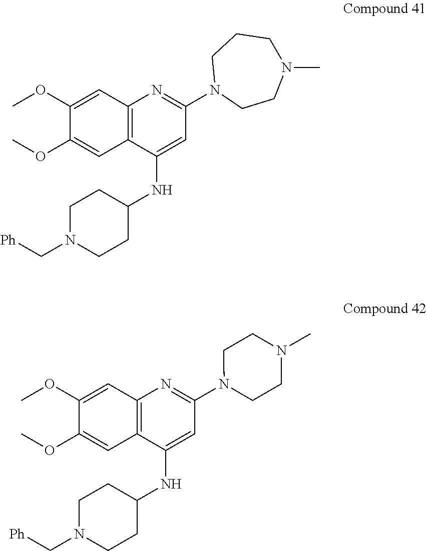

The selectivity of these compounds was examined in a methyltransferase enzyme panel including one DNA methyltransferase (DNMT). According to the authors, compounds 41 and 42 were found to be inactive against the DNA methyltransferase.

There is still a need of developing compounds which show improved activity in the treatment and/or prevention of cancer, fibrosis and immunomodulation.

### SUMMARY OF THE INVENTION

Inventors have found new compounds having a 2,4,6,7-tetrasubstituted quinoline core which are capable to inhibit one or more DNA methyltransferases (DNMTs, including DNMT1, DNMT3A and/or DNMT3B) as demonstrated by the examples of the invention. These compounds are therefore inhibitors of DNMTs and could be useful for the treatment and/or prevention of cancer, fibrosis and/or immunomodulation.

Further, some compounds of the invention are also capable to inhibit the histone methyltransferase G9a being dual inhibitors. Regarding their use in cancer, these compounds of the invention have the advantage that they are addressed to two different targets of those that, in in vitro tests, cell-based assays or in animal models, have proved useful for the treatment of cancer. The fact that these compounds of the present invention have an impact on two pathophysiological events, may lead to a more efficacious treatment.

Therefore, a first aspect of the invention relates to a compound of formula (I), or a pharmaceutically or veterinary acceptable salt thereof, or any stereoisomer or mixtures of stereoisomers, either of the compound of formula (I) or of any of its pharmaceutically or veterinary acceptable salts

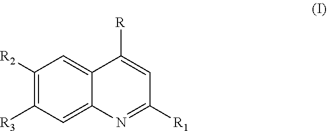

wherein

R is a radical selected from the group consisting of formula (A), formula (B), formula (C), formula (D), and formula (E):

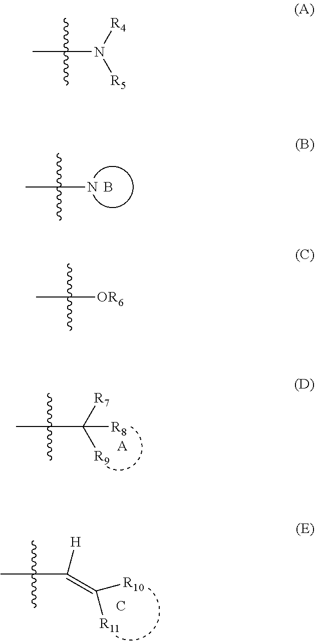

R₁ is a known ring system selected from the group consisting of:
(i) phenyl;
(ii) 5- or 6-membered heteroaromatic ring;
(iii) 3- to 7-membered saturated or partially unsaturated carbocyclic or heterocyclic monocyclic ring;
(iv) 3- to 7-membered saturated or partially unsaturated or aromatic carbocyclic or heterocyclic monocyclic ring, which is fused, bridged-fused or spiro-fused to a 3- to 7-membered saturated or partially unsaturated or aromatic carbocyclic or heterocyclic monocyclic ring;
(v) phenyl fused to a 6- to 14-membered saturated or partially unsaturated carbocyclic or heterocyclic bicyclic ring, wherein the rings of the bicyclic ring are spiro-fused; and
(vi) 5- to 6-membered heteroaromatic ring fused to a 6- to 14-membered saturated or partially unsaturated carbocyclic or heterocyclic bicyclic ring, wherein the rings of the bicyclic ring are spiro-fused;
wherein R₁ is optionally substituted with:
   a) one Cy¹ or one Cy², and/or
   b) one or more substituents R^{a}, and/or
   c) one or more substituents Z¹ optionally substituted with one or more substituents R^{a} and/or one Cy¹;
   wherein Cy¹ or Cy² are optionally substituted with one or more substituents independently selected from R^{a}, and Z² optionally substituted with one or more substituents R^{a};

R₂ is selected from the group consisting of R^{b}, halogen, —NO₂, —CN, —OR^{b}, —OC(O)R^{b′}, —OC(O)OR^{b′}, —OC(O)NR^{b′}R^{b′}, —NR^{b′}R^{b′}, —NR^{b′}C(O)R^{b′} —NR^{b′}C(O)OR^{b′}, —NR^{b′}C(O)NR^{b′}R^{b′}, —NR^{b′}S(O)₂R^{b′}, —NR^{b′}SO₂NR^{b′}R^{b′}, —SR^{b′}, —S(O)R^{b′}, —S(O)OR^{b′}, —SO₂R^{b′}, —SO₂(OR^{b′}), —SO₂NR^{b′}R^{b′}, —SC(O)NR^{b′}R^{b′} —C(O)R^{b′}, —C(O)OR^{b′}, —C(O)NR^{b′}R^{b′}, —C(O)NR^{b′}OR^{b′}, and —C(O)NR^{b′}SO₂R^{b′};

R₃ is selected from the group consisting of R^{c}, —OR^{d}, —OR^{e}, —NR^{b′}R^{d}, —NR^{b′}R^{e}, —NR^{f}COR^{d}, and —NR^{f}COR^{e};

R₄ and R₆ are independently selected from the group consisting of Cy¹, and Z¹ optionally substituted with one or more substituents R^{a} and/or one Cy³; wherein Cy¹ is optionally substituted with:
a) one Cy²; and/or
b) one or more substituents R^{a}, and/or
c) one or more substituents Z² optionally substituted with one or more substituents R^{a} and/or one Cy²;
wherein Cy² and Cy³ are optionally substituted with one or more substituents independently selected from R^{a}, and Z³ optionally substituted with one or more substituents R^{a};

R₅ is (C₁-C₆)alkyl optionally substituted with one or more halogen atoms or a 3- to 7-membered saturated or partially unsaturated carbocyclic monocyclic ring optionally substituted with one or more halogen atoms;

the dotted line means the presence or absence of a ring system A or C;

R₇ is absent or is selected from the group consisting of H, R^{a}, Cy¹, and Z¹ optionally substituted with one or more substituents R^{a} and/or one Cy³; wherein Cy¹ is optionally substituted with:
a) one Cy²; and/or
a) one or more substituents R^{a}, and/or
b) one or more substituents Z² optionally substituted with one or more substituents R^{a} and/or one Cy²;
wherein Cy² and Cy³ are optionally substituted with one or more substituents independently selected from R^{a}, and Z³ optionally substituted with one or more substituents R^{a};

R₈ and R₉ are independently selected from the group consisting of H, halogen, (C₁-C₆)alkyl optionally substituted with one or more halogen atoms, and a 3- to 7-membered saturated or partially unsaturated carbocyclic monocyclic ring optionally substituted with one or more halogen atoms; or alternatively

R₈ and R₉, together with the carbon atom to which they are attached, form a known ring system A comprising a 3- to 7-membered saturated or partially unsaturated carbocyclic or heterocyclic monocyclic ring, which is optionally fused, bridged-fused or spiro-fused to a 3- to 7-membered saturated or partially unsaturated or aromatic carbocyclic or heterocyclic monocyclic ring; wherein the ring system A is optionally substituted with:
a) one Cy¹; and/or
b) one or more substituents R^{a}, and/or
c) one or more substituents Z¹ optionally substituted with one or more substituents R^{a} and/or one Cy³;
wherein Cy¹ and Cy³ are optionally substituted with one or more substituents independently selected from R^{a}, and Z² optionally substituted with one or more substituents R^{a};

R₁₀ and R₁₁ are independently selected from the group consisting of H, and Z¹ optionally substituted with one or more substituents R^{a} and/or one Cy³; wherein Cy³ are optionally substituted with one or more substituents independently selected from R^{a}, and Z³ optionally substituted with one or more substituents R^{a}; or alternatively

R₁₀ and R₁₁, together with the carbon atom to which they are attached, form a known ring system C comprising a 3- to 7-membered saturated or partially unsaturated carbocyclic or heterocyclic monocyclic ring, which is optionally fused, bridged-fused or spiro-fused to a 3- to 7-membered saturated or partially unsaturated or aromatic carbocyclic or heterocyclic monocyclic ring; wherein the ring system C is optionally substituted with:
a) one Cy¹; and/or
b) one or more substituents R^{a}, and/or
c) one or more substituents Z¹ optionally substituted with one or more substituents R^{a} and/or one Cy³;
wherein Cy¹ and Cy³ are optionally substituted with one or more substituents independently selected from R^{a}, and Z² optionally substituted with one or more substituents R^{a};

B is a known ring system comprising a 3- to 7-membered saturated or partially unsaturated heterocyclic monocyclic ring, which is optionally fused, bridged-fused or spiro-fused to a 3- to 7-membered saturated or partially unsaturated or aromatic carbocyclic or heterocyclic monocyclic ring; wherein the ring system B is optionally substituted with:
a) one Cy¹; and/or
b) one or more substituents R^{a}, and/or
c) one or more substituents Z¹ optionally substituted with one or more substituents R^{a} and/or one Cy³;
wherein Cy¹ and Cy³ are optionally substituted with one or more substituents independently selected from R^{a}, and Z² optionally substituted with one or more substituents R^{a};

each R^{a} is independently selected from the group consisting of halogen, —NO₂, —CN, —OR^{b′}, —OC(Y)R^{b′}, —OC(Y)OR^{b}, —OC(Y)NR^{b′}R^{b′}, —NR^{b′}R^{b′}—NR^{b′}C(Y)R^{b′}, —NR^{b′}C(Y)OR^{b′}, —NR^{b′}C(Y)NR^{b′}R^{b′}, —NR^{b′}S(O)₂R^{b′}, —NR^{b′}SO₂NR^{b′}R^{b′}, —SR^{b′}, —S(O)R^{b′}, —S(O)OR^{b′}, —SO₂R^{b′}, —SO₂(OR^{b′}), —SO₂NR^{b′}R^{b′}, —SC(Y)NR^{b′}R^{b′}, —C(Y)R^{b′}, —C(Y)OR^{b′}, —C(Y)NR^{b′}R^{b′}, —C(Y)NR^{b′}OR^{b′}, and —C(O)NR^{b′}SO₂R^{b′};

each R^{b′} is independently H or R^{b};

each R^{b} is independently selected from the group consisting of (C₁-C₆)alkyl, (C₂-C₆)alkenyl, (C₂-C₆)alkynyl, (C₂-C₆)hydrocarbon chain having one or more double bonds and one or more triple bonds, and 3- to 7-membered saturated or partially unsaturated or aromatic carbocyclic or heterocyclic monocyclic ring, wherein each R^{b} is optionally substituted with one or more halogen atoms,

R^{c} is R^{d} or R^{g}; with the proviso that R^{c} is a moiety comprising at least one heteroatom selected from N, O, S, and F;

R^{d} is Cy¹ optionally substituted with:
a) one Cy²; and/or
b) one or more substituents R^{a}, and/or
c) one or more substituents Z¹ optionally substituted with one or more substituents R^{a} and/or one Cy²;
wherein Cy² is optionally substituted with one or more substituents independently selected from R^{a}, and Z² optionally substituted with one or more substituents R^{a};

R^{e} is a moiety comprising at least 4 carbon atoms which is selected from the group consisting of (C₁-C₁₂)alkyl, (C₂-C₁₂)alkenyl, (C₂-C₁₂)alkynyl, and (C₂-C₆)hydrocarbon chain having one or more double bonds and one or more triple bonds; wherein R^{e} is optionally substituted with one or more substituents R^{a} and/or one Cy³; wherein Cy³ is optionally substituted with:
a) one Cy⁴; and/or
b) one or more substituents R^{a}, and/or
c) one or more substituents Z³ optionally substituted with one or more substituents Ra and/or one Cy⁴;
wherein Cy⁴ is optionally substituted with one or more substituents independently selected from R^{a}, and Z⁴ optionally substituted with one or more substituents R^{a};

R^{f} is H or R;

R^{f} is selected from the group consisting of (C₁-C₆)alkyl, (C₂-C₆)alkenyl, (C₂-C₆)alkynyl, (C₂-C₆)hydrocarbon chain having one or more double bonds and one or more triple bonds, wherein each R^{f} is optionally substituted with one or more halogen atoms;

R^{g} is selected from the group consisting of (C₁-C₁₂)alkyl, (C₂-C₁₂)alkenyl, (C₂-C₁₂)alkynyl, and (C₂-C₆)hydrocarbon chain having one or more double bonds and one or more triple bonds; wherein R^{g} is optionally substituted with one or more substituents R^{a} and/or one Cy³; wherein Cy³ is optionally substituted with:
a) one Cy⁴; and/or
b) one or more substituents R^{a}, and/or
c) one or more substituents Z³ optionally substituted with one or more substituents R^{a} and/or one Cy⁴;
wherein Cy⁴ is optionally substituted with one or more substituents independently selected from R^{a}, and Z⁴ optionally substituted with one or more substituents R^{a};

Y is O, S, or NR^{b′};

Z¹, Z², Z³ and Z⁴ are independently selected from the group consisting of (C₁-C₁₂)alkyl, (C₂-C₁₂)alkenyl, (C₂-C₁₂)alkynyl, and (C₂-C₆)hydrocarbon chain having one or more double bonds and one or more triple bonds;

Cy¹ and Cy³ are independently a known ring system selected from the group consisting of phenyl; 5- or 6-membered heteroaromatic ring; 3- to 7-membered saturated or partially unsaturated carbocyclic or heterocyclic monocyclic ring; and 3- to 7-membered saturated or partially unsaturated or aromatic carbocyclic or heterocyclic monocyclic ring, which is fused, bridged-fused or spiro-fused to a 3- to 7-membered saturated or partially unsaturated or aromatic carbocyclic or heterocyclic monocyclic ring;

Cy², Cy⁴ are independently a known ring system selected from the group consisting of phenyl; 3- to 7-membered saturated or partially unsaturated carbocyclic or heterocyclic monocyclic ring; and 5- or 6-membered heteroaromatic ring;

wherein in the carbocyclic rings all ring members are carbon atoms; and in the heterocyclic and heteroaromatic rings one or more ring members are selected from N, O, and S; and wherein in all saturated or partially unsaturated rings one or two members of the rings are optionally C(O) and/or C(NH) and/or C[N(C₁-C₄)alkyl];

with the proviso that the compound of formula (I) is other than: 2,2,2-trifluoro-N-[4-(methoxymethyl)-6-methyl-2-(4-phenyl-1-piperazinyl)-7-quinolinyl]acetamide.

A second aspect of the invention relates to a compound of formula (I), or a pharmaceutically or veterinary acceptable salt thereof, or any stereoisomer or mixtures of stereoisomers, either of the compound of formula (I) or of any of its pharmaceutically or veterinary acceptable salts as defined in the first aspect, wherein R^{e} is a moiety comprising at least 5 carbon atoms.

A third aspect of the invention relates to a compound of formula (I), or a pharmaceutically or veterinary acceptable salt thereof, or any stereoisomer or mixtures of stereoisomers, either of the compound of formula (I) or of any of its pharmaceutically or veterinary acceptable salts

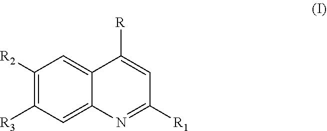

wherein

R is a radical selected from the group consisting of formula (A), formula (B), formula (C), formula (D), and formula (E):

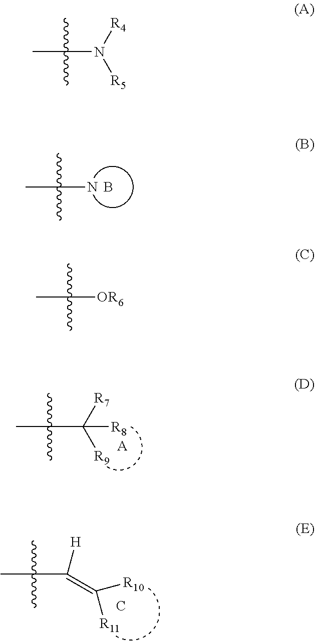

R₁ is a known ring system attached to the quinoline ring through a carbon atom, which is selected from the group consisting of:
(i) phenyl;
(ii) 5- or 6-membered heteroaromatic ring;
(iii) 3- to 7-membered saturated or partially unsaturated carbocyclic or heterocyclic monocyclic ring;
(iv) 3- to 7-membered saturated or partially unsaturated or aromatic carbocyclic or heterocyclic monocyclic ring, which is fused, bridged-fused or spiro-fused to a 3- to 7-membered saturated or partially unsaturated or aromatic carbocyclic or heterocyclic monocyclic ring;
(v) phenyl fused to a 6- to 14-membered saturated or partially unsaturated carbocyclic or heterocyclic bicyclic ring, wherein the rings of the bicyclic ring are spiro-fused; and
(vi) 5- to 6-membered heteroaromatic ring fused to a 6- to 14-membered saturated or partially unsaturated carbocyclic or heterocyclic bicyclic ring, wherein the rings of the bicyclic ring are spiro-fused;
wherein R₁ is optionally substituted with:
   a) one Cy¹ or one Cy², and/or
   b) one or more substituents R^{a}, and/or
   c) one or more substituents Z¹ optionally substituted with one or more substituents R^{a} and/or one Cy¹;
   wherein Cy¹ or Cy² are optionally substituted with one or more substituents independently selected from R^{a}, and Z² optionally substituted with one or more substituents R^{a};

R₂ is selected from the group consisting of R^{b}, halogen, —NO₂, —CN, —OR^{b}, —OC(O)R^{b′}, —OC(O)OR^{b′}, —OC(O)NR^{b′}R^{b′}, —NR^{b′}R^{b′}, —NR^{b′}C(O)R^{b′} —NR^{b′}C(O)OR^{b′}, —NR^{b′}C(O)NR^{b′}R^{b′}, —NR^{b′}S(O)₂R^{b′}, —NR^{b′}SO₂NR^{b′}R^{b′}, —SR^{b′} —S(O)R^{b′}, —S(O)OR^{b′}, —SO₂R^{b′}, —SO₂(OR^{b′}), —SO₂NR^{b′}R^{b′}, —SC(O)NR^{b′}R^{b′} —C(O)R^{b′}, —C(O)OR^{b′}, —C(O)NR^{b′}R^{b′}, —C(O)NR^{b′}OR^{b′}, and —C(O)NR^{b′}SO₂R^{b′};

R₃ is selected from the group consisting of —OR^{d} and —OR^{e};

R₄ and R₆ are independently selected from the group consisting of Cy¹, and Z¹ optionally substituted with one or more substituents R^{a} and/or one Cy³; wherein Cy¹ is optionally substituted with:
a) one Cy²; and/or
b) one or more substituents R^{a}, and/or
c) one or more substituents Z² optionally substituted with one or more substituents R^{a} and/or one Cy²;
wherein Cy² and Cy³ are optionally substituted with one or more substituents independently selected from R^{a}, and Z³ optionally substituted with one or more substituents R^{a};

R₅ is (C₁-C₆)alkyl optionally substituted with one or more halogen atoms or a 3- to 7-membered saturated or partially unsaturated carbocyclic monocyclic ring optionally substituted with one or more halogen atoms;

the dotted line means the presence or absence of a ring system A or C;

R₇ is absent or is selected from the group consisting of H, R^{a}, Cy¹, and Z¹ optionally substituted with one or more substituents R^{a} and/or one Cy³; wherein Cy¹ is optionally substituted with:
a) one Cy²; and/or
b) one or more substituents R^{a}, and/or
c) one or more substituents Z² optionally substituted with one or more substituents R^{a} and/or one Cy²;
wherein Cy² and Cy³ are optionally substituted with one or more substituents independently selected from R^{a}, and Z³ optionally substituted with one or more substituents R^{a};

R₈ and R₉ are independently selected from the group consisting of H, halogen, (C₁-C₆)alkyl optionally substituted with one or more halogen atoms, and a 3- to 7-membered saturated or partially unsaturated carbocyclic monocyclic ring optionally substituted with one or more halogen atoms; or alternatively

R₈ and R₉, together with the carbon atom to which they are attached, form a known ring system A comprising a 3- to 7-membered saturated or partially unsaturated carbocyclic or heterocyclic monocyclic ring, which is optionally fused, bridged-fused or spiro-fused to a 3- to 7-membered saturated or partially unsaturated or aromatic carbocyclic or heterocyclic monocyclic ring; wherein the ring system A is optionally substituted with:
a) one Cy¹; and/or
b) one or more substituents R^{a}, and/or
c) one or more substituents Z¹ optionally substituted with one or more substituents R^{a} and/or one Cy³;
wherein Cy¹ and Cy³ are optionally substituted with one or more substituents independently selected from R^{a}, and Z² optionally substituted with one or more substituents R^{a};

R₁₀ and R₁₁ are independently selected from the group consisting of H, and Z¹ optionally substituted with one or more substituents R^{a} and/or one Cy³; wherein Cy³ are optionally substituted with one or more substituents independently selected from R^{a}, and Z³ optionally substituted with one or more substituents R^{a}; or alternatively

R₁₀ and R₁₁, together with the carbon atom to which they are attached, form a known ring system C comprising a 3- to 7-membered saturated or partially unsaturated carbocyclic or heterocyclic monocyclic ring, which is optionally fused, bridged-fused or spiro-fused to a 3- to 7-membered saturated or partially unsaturated or aromatic carbocyclic or heterocyclic monocyclic ring; wherein the ring system C is optionally substituted with:
a) one Cy¹; and/or
b) one or more substituents R^{a}, and/or
c) one or more substituents Z¹ optionally substituted with one or more substituents R^{a} and/or one Cy³;
wherein Cy¹ and Cy³ are optionally substituted with one or more substituents independently selected from R^{a}, and Z² optionally substituted with one or more substituents R^{a};

B is a known ring system comprising a 3- to 7-membered saturated or partially unsaturated heterocyclic monocyclic ring, which is optionally fused, bridged-fused or spiro-fused to a 3- to 7-membered saturated or partially unsaturated or aromatic carbocyclic or heterocyclic monocyclic ring; wherein the ring system B is optionally substituted with:
a) one Cy¹; and/or
b) one or more substituents R^{a}, and/or
c) one or more substituents Z¹ optionally substituted with one or more substituents R^{a} and/or one Cy³;
wherein Cy¹ and Cy³ are optionally substituted with one or more substituents independently selected from R^{a}, and Z² optionally substituted with one or more substituents R^{a};

each R^{a} is independently selected from the group consisting of halogen, —NO₂, —CN, —OR^{b′}, —OC(Y)R^{b′}, —OC(Y)OR^{b}, —OC(Y)NR^{b′}R^{b′}, —NR^{b′}R^{b′}—NR^{b′}C(Y)R^{b′}, —NR^{b′}C(Y)OR^{b′}, —NR^{b′}C(Y)NR^{b′}R^{b′}, —NR^{b′}S(O)₂R^{b′}, —NR^{b′}SO₂NR^{b′}R^{b′}, —SR^{b′}, —S(O)R^{b′}, —S(O)OR^{b′}, —SO₂R^{b′}, —SO₂(OR^{b′}), —SO₂NR^{b′}R^{b′}, —SC(Y)NR^{b′}R^{b′}, —C(Y)R^{b′}, —C(Y)OR^{b′}, —C(Y)NR^{b′}R^{b′}, —C(Y)NR^{b′}OR^{b′}, and —C(O)NR^{b′}SO₂R^{b′};

each R^{b′} is independently H or R^{b};

each R^{b} is independently selected from the group consisting of (C₁-C₆)alkyl, (C₂-C₆)alkenyl, (C₂-C₆)alkynyl, (C₂-C₆)hydrocarbon chain having one or more double bonds and one or more triple bonds, and 3- to 7-membered saturated or partially unsaturated or aromatic carbocyclic or heterocyclic monocyclic ring, wherein each R^{b} is optionally substituted with one or more halogen atoms,

R^{d} is Cy¹ optionally substituted with:
a) one Cy²; and/or
b) one or more substituents R^{a}, and/or
c) one or more substituents Z¹ optionally substituted with one or more substituents R^{a} and/or one Cy²;
wherein Cy² is optionally substituted with one or more substituents independently selected from R^{a}, and Z² optionally substituted with one or more substituents R^{a};

R^{e} is a moiety comprising at least 4 carbon atoms which is selected from the group consisting of (C₁-C₁₂)alkyl, (C₂-C₁₂)alkenyl, (C₂-C₁₂)alkynyl, and (C₂-C₆)hydrocarbon chain having one or more double bonds and one or more triple bonds; wherein R^{e} is optionally substituted with one or more substituents R^{a} and/or one Cy³; wherein Cy³ is optionally substituted with:
a) one Cy⁴; and/or
b) one or more substituents R^{a}, and/or
c) one or more substituents Z³ optionally substituted with one or more substituents Ra and/or one Cy⁴;
wherein Cy⁴ is optionally substituted with one or more substituents independently selected from R^{a}, and Z⁴ optionally substituted with one or more substituents R^{a};

Y is O, S, or NR^{b′};

Z¹, Z², Z³ and Z⁴ are independently selected from the group consisting of (C₁-C₁₂)alkyl, (C₂-C₁₂)alkenyl, (C₂-C₁₂)alkynyl, and (C₂-C₆)hydrocarbon chain having one or more double bonds and one or more triple bonds;

Cy¹ and Cy³ are independently a known ring system selected from the group consisting of phenyl; 5- or 6-membered heteroaromatic ring; 3- to 7-membered saturated or partially unsaturated carbocyclic or heterocyclic monocyclic ring; and 3- to 7-membered saturated or partially unsaturated or aromatic carbocyclic or heterocyclic monocyclic ring, which is fused, bridged-fused or spiro-fused to a 3- to 7-membered saturated or partially unsaturated or aromatic carbocyclic or heterocyclic monocyclic ring;

Cy², Cy⁴ are independently a known ring system selected from the group consisting of phenyl; 3- to 7-membered saturated or partially unsaturated carbocyclic or heterocyclic monocyclic ring; and 5- or 6-membered heteroaromatic ring;

wherein in the carbocyclic rings all ring members are carbon atoms; and in the heterocyclic and heteroaromatic rings one or more ring members are selected from N, O, and S; and wherein in all saturated or partially unsaturated rings one or two members of the rings are optionally C(O) and/or C(NH) and/or C[N(C₁-C₄)alkyl].

A fourth aspect of the invention relates to a compound of formula (I), or a pharmaceutically or veterinary acceptable salt thereof, or any stereoisomer or mixtures of stereoisomers, either of the compound of formula (I) or of any of its pharmaceutically or veterinary acceptable salts as defined in the third aspect, wherein R^{e} is a moiety comprising at least 5 carbon atoms.

Another aspect of the invention relates to a pharmaceutical or veterinary composition which comprises an effective amount of a compound of formula (I) as defined above, or a pharmaceutically or veterinary acceptable salt thereof, or any stereoisomer or mixtures of stereoisomers, either of the compound of formula (I) or of its pharmaceutically or veterinary acceptable salt, together with one or more pharmaceutically or veterinary acceptable excipients or carriers.

Another aspect of the invention relates to a compound of formula (I) or a pharmaceutical or veterinary composition as defined above, for use in the treatment and/or prevention of cancer, fibrosis and/or immunomodulation. Thus, this aspect of the invention relates to the use of a compound of formula (I) as defined above, for the manufacture of a medicament for the treatment and/or prevention of cancer, fibrosis and/or immunomodulation; and may also be formulated as a method for the treatment and/or prevention of cancer, fibrosis and/or immunomodulation, comprising administering an effective amount of the previously defined compound of formula (I) as defined above, and one or more pharmaceutically or veterinary acceptable excipients or carriers, in a subject in need thereof, including a human.

### DETAILED DESCRIPTION OF THE INVENTION

All terms as used herein in this application, unless otherwise stated, shall be understood in their ordinary meaning as known in the art. Other more specific definitions for certain terms as used in the present application are as set forth below and are intended to apply uniformly through-out the specification and claims unless an otherwise expressly set out definition provides a broader definition.

The term “carbocyclic” ring system refers to a known ring system wherein all the ring members contain carbon atoms. The term “heterocyclic” ring system refers to a known ring system wherein one or more of the ring members, preferably 1, 2, 3, or 4 ring members, are selected from NH, N, O, and S, where chemically possible. The remaining ring members of the heterocyclic ring are independently selected from C, CH, CH₂, O, N, NH, and S. Unless otherwise specified, the “heterocyclic” ring system may be attached to the rest of the molecule through a C or a N atom of the ring system. Both the carbocyclic and heterocyclic rings can be saturated, partially unsaturated, or aromatic and may be unsubstituted or substituted as described herein, being the substituents placed on any available position. Thus, in a ring member of a carbocyclic ring that is CH or CH₂ or in a ring member of a heterocyclic ring that is CH, CH₂ or NH, one or more of the H atoms of these ring members may be substituted by another moiety as herein disclosed.

For the purposes of the present invention, in “fused” rings the fusion occurs through one bond which is common to two adjoining rings; in “bridged-fused” rings the fusion occurs through a sequence of atoms (bridgehead) which is common to two rings; and in “spiro-fused” rings, the fusion occurs through only one atom (spiro atom), preferably a carbon atom, which is common to two adjoining rings (including bridged rings).

The term “heteroaromatic” ring refers to a known aromatic ring system, wherein one or more of the ring members, preferably 1, 2, 3, or 4 ring members, are selected from NH, N, O, and S, where chemically possible. The remaining ring members of the heteroaromatic ring are independently selected from C, CH, O, N, NH, and S. The heteroaromatic ring may be unsubstituted or substituted as described herein, being the substituents placed on any available position. Thus, in a ring member of the heteroaromatic ring which is CH or NH the H atom may be substituted by another moiety, as herein disclosed.

The present invention also includes the tautomeric forms of the compounds of formula (I). The term “tautomeric isomers” means isomers, the structures of which differ in the position of an atom, generally a hydrogen atom, and of one or more multiple bonds, and which are capable of easily and reversibly changing from one to another. The tautomers are used indistinctly in the present application. Thus, as an example, a hydroxyphenyl group has to be considered equivalent to its tautomeric form: cyclohexa-2,4-dienone. All tautomers are to be considered equivalent for the purposes of the invention.

The term “known ring system” as used herein refers to a ring system which is chemically feasible and is known in the art and so intends to exclude those ring systems that are not chemically possible.

For the purposes of the present invention, in all saturated or partially unsaturated rings, one or two members of the rings are optionally C(O) and/or C(NH) and/or C[N(C₁-C₄)alkyl].

The term (C₁-Cₙ)alkyl refers to a saturated branched or linear hydrocarbon chain which contains from 1 to n carbon atoms and only single bonds. The term (C₂-Cₙ)alkenyl refers to an unsaturated branched or linear hydrocarbon chain which comprises from 2 to n carbon atoms and at least one or more double bonds. The term (C₂-Cₙ)alkynyl refers to a saturated branched or linear hydrocarbon chain which comprises from 2 to n carbon atoms and at least one or more triple bonds. For the purposes of the invention, the (C₂-Cₙ)hydrocarbon chain having one or more double bonds and one or more triple bonds is a branched or linear hydrocarbon chain which contains from 2 to n carbon atoms.

A halogen substituent means fluoro, chloro, bromo or iodo.

In the embodiments of the invention referring to the compounds of formula (I), where the substitution or unsubstitution of a certain group is not specified, e.g. either by indicating a certain substitution for that group or by indicating that the group is unsubstituted, it has to be understood that the possible substitution of this group is the one as in the definition of the formula (I).

Further, the expression “substituted as defined herein”, “substituted as previously defined”, “substituted as defined above” or any equivalent expression has to be understood that the possible substitution of this group is the one as in the definition of the formula (I).

“Protective group” (PG) refers to a grouping of atoms that when attached to a reactive group in a molecule masks, reduces or prevents that reactivity.

The expression “substituted with one or more” means that a group can be substituted with one or more, preferably with 1, 2, 3 or 4 substituents, provided that this group has enough positions susceptible of being substituted.

For the purposes of the invention, room temperature is 20-25 OC.

In the first and second aspects of the invention related to the compounds of formula (I), the compound of the invention is other than 2,2,2-trifluoro-N-[4-(methoxymethyl)-6-methyl-2-(4-phenyl-1-piperazinyl)-7-quinolinyl]acetamide (CAS RN: 866134-27-2), having the following chemical formula:

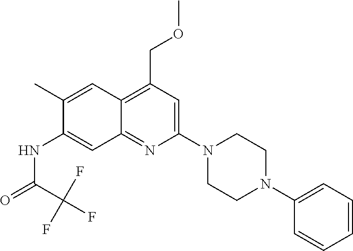 [SMILES: COCc1cc(N2CCN(c3ccccc3)CC2)nc2cc(NC(=O)C(F)(F)F)c(C)cc12]

This compound is a commercial product with no associated bibliographic references.

There is no limitation on the type of salt of the compounds of the invention that can be used, provided that these are pharmaceutically or veterinary acceptable when they are used for therapeutic purposes. The term “pharmaceutically or veterinary acceptable salts”, embraces salts commonly used to form alkali metal salts and to form addition salts of free acids or free bases.

The preparation of pharmaceutically or veterinary acceptable salts of the compounds of formula (I) can be carried out by methods known in the art. For instance, they can be prepared from the parent compound, which contains a basic or acidic moiety, by conventional chemical methods. Generally, such salts are, for example, prepared by reacting the free acid or base forms of these compounds with a stoichiometric amount of the appropriate pharmaceutically or veterinary acceptable base or acid in water or in an organic solvent or in a mixture of them. The compounds of formula (I) and their salts may differ in some physical properties but they are equivalent for the purposes of the present invention.

The compounds of the invention may be in crystalline form either as free solvation compounds or as solvates (e.g. hydrates) and it is intended that both forms are within the scope of the present invention. Methods of solvation are generally known within the art. In general, the solvated forms with pharmaceutically or veterinary acceptable solvents such as water, ethanol and the like are equivalent to the unsolvated form for the purposes of the invention.

Some compounds of the invention can have chiral centres that can give rise to various stereoisomers. As used herein, the term “stereoisomer” refers to all isomers of individual compounds that differ only in the orientation of their atoms in space. The term stereoisomer includes mirror image isomers (enantiomers), mixtures of mirror image isomers (racemates, racemic mixtures), geometric (cis/trans or syn/anti or E/Z) isomers, and isomers of compounds with more than one chiral center that are not mirror images of one another (diastereoisomers). The present invention relates to each of these stereoisomers and also mixtures thereof.

Diastereoisomers and enantiomers can be separated by conventional techniques such as chromatography or fractional crystallization. Optical isomers can be resolved by conventional techniques of optical resolution to give optically pure isomers. This resolution can be carried out on any chiral synthetic intermediates or on compounds of the invention. Optically pure isomers can also be individually obtained using enantiospecific synthesis.

In all embodiments of the invention referring to the compounds of formula (I), the pharmaceutically or veterinary acceptable salts thereof and the stereoisomers or mixtures of stereoisomers, either of any of the compounds of formula (I) or of any of their pharmaceutically acceptable salts are always contemplated even if they are not specifically mentioned.

In one embodiment, optionally in combination with one or more features of the various embodiments described above or below, the invention relates to a compound of formula (I) as previously described, wherein R is a radical selected from the group consisting of formula (A) (i.e. a compound (IA)) and formula (B) (i.e. a compound (IB)):

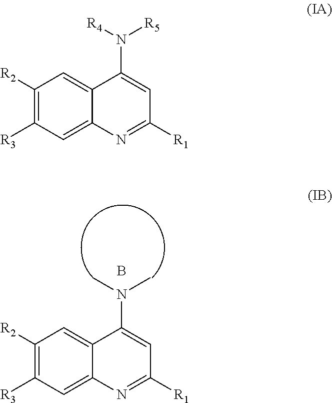

In another embodiment, optionally in combination with one or more features of the various embodiments described above or below, the invention relates to a compound of formula (I) as previously described, wherein R is a radical of formula (A) (i.e. a compound (IA)), and R₄ is Cy¹ optionally substituted with one or more substituents Z² optionally substituted as previously defined. More particularly, Cy¹ in R₄ is a known ring system selected from group consisting of 3- to 7-membered saturated or partially unsaturated carbocyclic or heterocyclic monocyclic ring; and 3- to 7-membered saturated or partially unsaturated carbocyclic or heterocyclic monocyclic ring, which is fused, bridged-fused or spiro-fused, more particularly bridged-fused or spiro-fused, to a 3- to 7-membered saturated or partially unsaturated carbocyclic or heterocyclic monocyclic ring, wherein Cy¹ is optionally substituted as previously defined. Even more particularly, Z² in R₄ is (C₁-C₆)alkyl optionally substituted with one or more substituents R^{a}.

In another embodiment, optionally in combination with one or more features of the various embodiments described above or below, the invention relates to a compound of formula (I) as previously described, wherein R is a radical of formula (A) (i.e. a compound (IA)), and R₄ is Z¹; more particularly Z¹ is (C₁-C₆)alkyl, optionally substituted as previously defined. Even more particularly, Z¹ in R₄ is substituted with Cy³, wherein Cy³ is optionally substituted as previously defined. Even more particularly, Cy³ in R₄ is a known ring system selected from phenyl; 5- or 6-membered heteroaromatic ring; 3- to 7-membered saturated or partially unsaturated carbocyclic or heterocyclic monocyclic ring; and 3- to 7-membered saturated or partially unsaturated carbocyclic or heterocyclic monocyclic ring, which is fused, bridged-fused or spiro-fused to a 3- to 7-membered saturated or partially unsaturated carbocyclic or heterocyclic monocyclic ring, wherein Cy³ is optionally substituted as previously defined. Even more particularly, Cy³ in R₄ is selected from 3- to 7-membered saturated or partially unsaturated carbocyclic or heterocyclic monocyclic ring; and 3- to 7-membered saturated or partially unsaturated carbocyclic or heterocyclic monocyclic ring, which is fused, bridged-fused or spiro-fused, more particularly bridged-fused or spiro-fused, to a 3- to 7-membered saturated or partially unsaturated carbocyclic or heterocyclic monocyclic ring, wherein Cy³ is optionally substituted as previously defined. Even more particularly, Cy³ is optionally substituted with one or more substituents Z³ optionally substituted as previously defined. More particularly, Z³ in R₄ is (C₁-C₆)alkyl optionally substituted with one or more substituents R^{a}.

In another embodiment, optionally in combination with one or more features of the various embodiments described above or below, the invention relates to a compound of formula (I) as previously described, wherein R is a radical of formula (A) (i.e. a compound (IA)), and R₄ is Z¹; more particularly Z¹ is (C₁-C₆)alkyl, optionally substituted with one or more halogen atoms.

In another embodiment, optionally in combination with one or more features of the various embodiments described above or below, the invention relates to a compound of formula (I) as previously described, wherein R is a radical of formula (A) (i.e. a compound (IA)), and R₅ is (C₁-C₆)alkyl optionally substituted with one or more halogen atoms, or a 3- to 7-membered saturated or partially unsaturated carbocyclic monocyclic ring optionally substituted with one or more halogen atoms. More particularly, R₅ is (C₁-C₆)alkyl optionally substituted with one or more halogen atoms, even more particularly is —CH₃.

In another embodiment, optionally in combination with one or more features of the various embodiments described above or below, the invention relates to a compound of formula (I) as previously described, wherein R is a radical of formula (A) (i.e. a compound (IA)) selected from the group consisting of the following moieties:

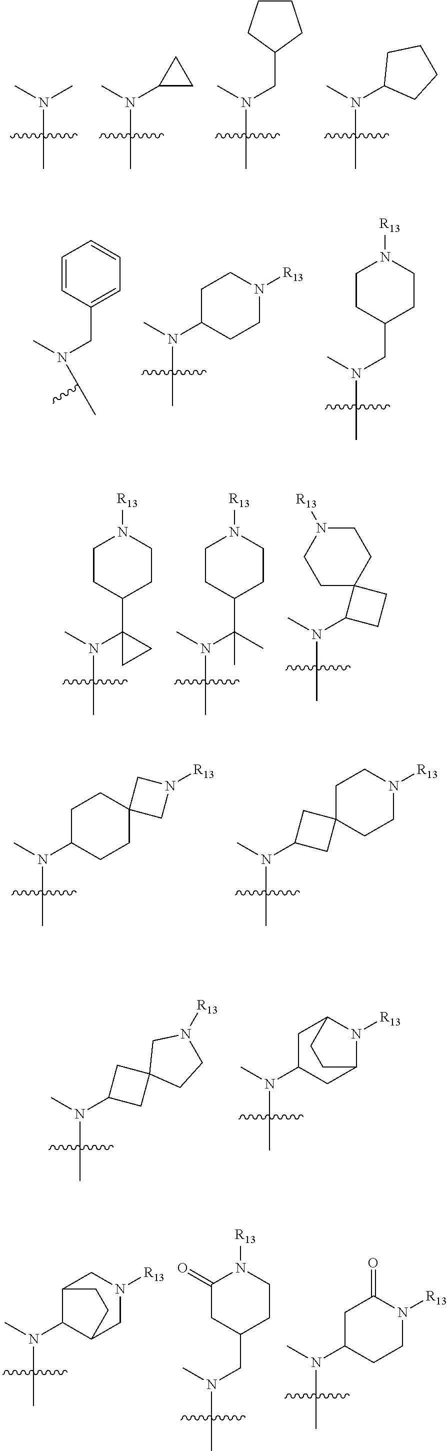

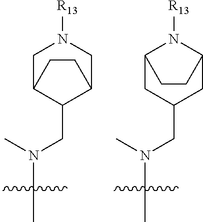

wherein R₁₃ is selected from the group consisting of H, methyl, isopropyl and cyclopropyl.

In another embodiment, optionally in combination with one or more features of the various embodiments described above or below, the invention relates to a compound of formula (I) as previously described, wherein R is a radical of formula (B) (i.e. a compound (IB)), and ring B is a known ring system comprising a 3- to 7-membered saturated or partially unsaturated heterocyclic monocyclic ring optionally substituted as previously defined or a 3- to 7-membered saturated or partially unsaturated carbocyclic or heterocyclic monocyclic ring, which is fused, bridged-fused or spiro-fused, more particularly bridged-fused or spiro-fused, to a 3- to 7-membered saturated or partially unsaturated carbocyclic or heterocyclic monocyclic ring optionally substituted as previously defined. More particularly, ring B is optionally substituted with: a) one or more substituents R^{a}, and/or b) one or more substituents Z¹; wherein Z¹ in ring B is optionally substituted as previously defined. Even more particularly, Z¹ in ring B is (C₁-C₆)alkyl optionally substituted with one or more substituents R^{a}.

In another embodiment, optionally in combination with one or more features of the various embodiments described above or below, the invention relates to a compound of formula (I) as previously described, wherein R is a radical of formula (B) (i.e. a compound (IB)) selected from the group consisting of the following moieties:

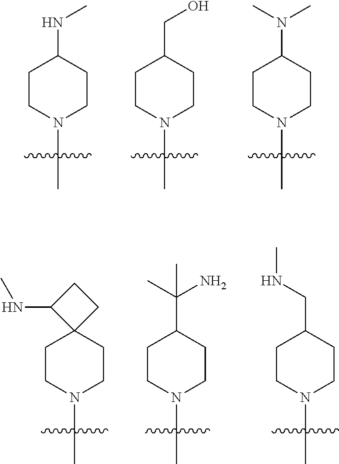

In another embodiment, optionally in combination with one or more features of the various embodiments described above or below, the invention relates to a compound of formula (I) as previously described, wherein R₁ is a known ring system selected from the group consisting of:
(i) phenyl;
(ii) 5- or 6-membered heteroaromatic ring;
(iii) 3- to 7-membered saturated or partially unsaturated carbocyclic or heterocyclic monocyclic ring; and
(iv) 3- to 7-membered saturated or partially unsaturated or aromatic carbocyclic or heterocyclic monocyclic ring, which is fused, bridged-fused or spiro-fused to a 3- to 7-membered saturated or partially unsaturated or aromatic carbocyclic or heterocyclic monocyclic ring;

wherein R₁ is optionally substituted as previously defined.

In another embodiment, optionally in combination with one or more features of the various embodiments described above or below, the invention relates to a compound of formula (I) as previously described, wherein R₁ is a known ring system selected from the group consisting of:
(ii) 5- or 6-membered heteroaromatic ring;
(iii) 3- to 7-membered saturated or partially unsaturated carbocyclic or heterocyclic monocyclic ring; more particularly 3- to 7-membered saturated or partially unsaturated heterocyclic monocyclic ring; even more particularly 3- to 7-membered saturated heterocyclic monocyclic ring;
(iv) 3- to 7-membered saturated or partially unsaturated or aromatic carbocyclic or heterocyclic monocyclic ring, which is fused, bridged-fused or spiro-fused to a 3- to 7-membered saturated or partially unsaturated or aromatic carbocyclic or heterocyclic monocyclic ring; more particularly 5- to 6-membered aromatic carbocyclic or heterocyclic monocyclic ring, which is fused to a 5- to 6-membered aromatic carbocyclic or heterocyclic monocyclic ring;

wherein R₁ is optionally substituted as previously defined.

In another embodiment, optionally in combination with one or more features of the various embodiments described above or below, the invention relates to a compound of formula (I) as previously described, wherein R₁ is a known ring system selected from the group consisting of:
(ii) 5- or 6-membered heteroaromatic ring; and
(iii) 3- to 7-membered saturated or partially unsaturated carbocyclic or heterocyclic monocyclic ring; more particularly 3- to 7-membered saturated or partially unsaturated heterocyclic monocyclic ring; even more particularly 3- to 7-membered saturated heterocyclic monocyclic ring;

wherein R₁ is optionally substituted as previously defined.

In another embodiment, optionally in combination with one or more features of the various embodiments described above or below, the invention relates to a compound of formula (I) as described in the first or second aspect, wherein R, is attached to the quinoline through a carbon atom.

In another embodiment, optionally in combination with one or more features of the various embodiments described above or below, the invention relates to a compound of formula (I) as previously described, wherein R₁ is a 5- to 6-membered heteroaromatic monocyclic ring, in particular attached to the quinoline through a carbon atom, and optionally substituted as previously defined. More particularly, R₁ is optionally substituted with one or more substituents Z¹, more particularly Z¹ is (C₁-C₁₂)alkyl, optionally substituted as previously defined.

In another embodiment, optionally in combination with one or more features of the various embodiments described above or below, the invention relates to a compound of formula (I) as previously described, wherein R₁ is selected from the group consisting of the following moieties:

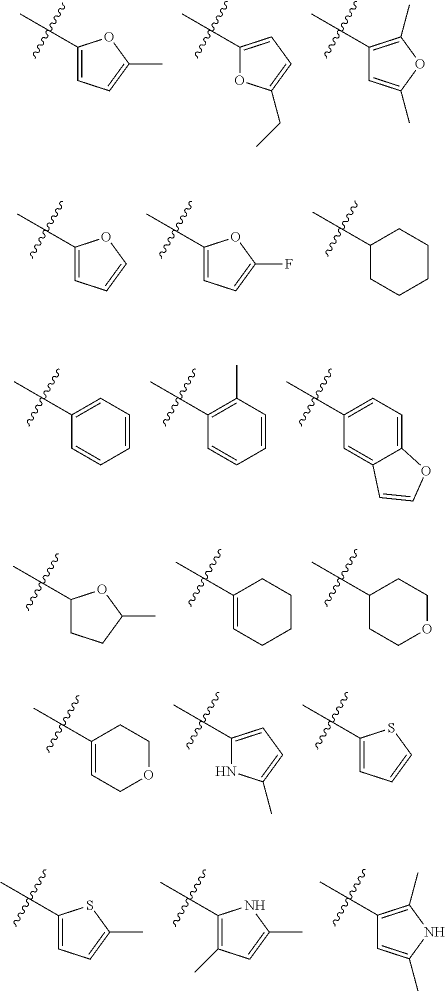

In another embodiment, optionally in combination with one or more features of the various embodiments described above or below, the invention relates to a compound of formula (I) as previously described, wherein R₂ is selected from halogen, —CN and —OR^{b′}, more particularly R₂ is selected from halogen and —OR^{b}; even more particularly R₂ is —OR^{b}; and even more particularly R^{b} in R₂ is (C₁-C₆)alkyl optionally substituted with one or more halogen atoms.

In another embodiment, optionally in combination with one or more features of the various embodiments described above or below, the invention relates to a compound of formula (I) as described in the first or second aspect, wherein R₃ is selected from the group consisting of —OR^{d}, —OR^{e}, —NR^{d}R^{b′}, and —NR^{e}R^{b′}. More particularly, R₃ is —OR^{d} or —OR^{e}, and even more particularly R^{d} or R^{e} in R₃ contains at least one N atom.

In another embodiment, optionally in combination with one or more features of the various embodiments described above or below, the invention relates to a compound of formula (I) as previously described, wherein R₃ is —OR^{e}. More particularly, R^{e} is (C₁-C₆)alkyl substituted as previously defined. Even more particularly R^{e} in —OR^{e} contains at least one N atom. Even more particularly, R^{e} is substituted with Cy³ as previously defined. Even more particularly, Cy³ in R^{e} is a known ring system selected from group consisting of a 3- to 7-membered saturated or partially unsaturated carbocyclic or heterocyclic monocyclic ring; and 3- to 7-membered saturated or partially unsaturated carbocyclic or heterocyclic monocyclic ring, which is fused, bridged-fused or spiro-fused, more particularly bridged-fused or spiro-fused, to a 3- to 7-membered saturated or partially unsaturated carbocyclic or heterocyclic monocyclic ring, wherein Cy³ is optionally substituted as previously defined. Even more particularly, Cy³ is a 3- to 7-membered saturated or partially unsaturated carbocyclic or heterocyclic monocyclic ring, and is optionally substituted as previously defined. Even more particularly, Cy³ is a 3- to 7-membered heterocyclic monocyclic ring and is optionally substituted with one or more substituents Z³ optionally substituted as previously defined. Even more particularly, Z³ in R^{e} is (C₁-C₆)alkyl substituted with one or more substituents R^{a}.

In another embodiment, optionally in combination with one or more features of the various embodiments described above or below, the invention relates to a compound of formula (I) as previously described, wherein R₃ is —OR^{d}, and R^{d} is Cy¹ optionally substituted with one or more substituents Z¹ optionally substituted as previously defined. More particularly, Cy¹ in R^{d} is a known ring system selected from group consisting of a 3- to 7-membered saturated or partially unsaturated carbocyclic or heterocyclic monocyclic ring; and 3- to 7-membered saturated or partially unsaturated carbocyclic or heterocyclic monocyclic ring, which is fused, bridged-fused or spiro-fused, more particularly bridged-fused or spiro-fused, to a 3- to 7-membered saturated or partially unsaturated carbocyclic or heterocyclic monocyclic ring, wherein Cy¹ is optionally substituted as previously defined. Even more particularly, Z¹ in R^{d} is (C₁-C₆)alkyl substituted with one or more substituents R^{a}.

In another embodiment, optionally in combination with one or more features of the various embodiments described above or below, the invention relates to a compound of formula (I) as described in the first or second aspect, wherein R₃ is —NR^{b′}R^{e}. More particularly, R^{e} is (C₁-C₆)alkyl substituted as previously defined. Even more particularly R^{e} in —NR^{b′}R^{e} contains at least one N atom.

Even more particularly, R^{e} is substituted with Cy³ as previously defined. Even more particularly, Cy³ in R^{e} is a known ring system selected from group consisting of a 3- to 7-membered saturated or partially unsaturated carbocyclic or heterocyclic monocyclic ring; and 3- to 7-membered saturated or partially unsaturated carbocyclic or heterocyclic monocyclic ring, which is fused, bridged-fused or spiro-fused, more particularly bridged-fused or spiro-fused, to a 3- to 7-membered saturated or partially unsaturated carbocyclic or heterocyclic monocyclic ring, wherein Cy³ is optionally substituted as previously defined. Even more particularly, Cy³ is a 3- to 7-membered saturated or partially unsaturated carbocyclic or heterocyclic monocyclic ring, and is optionally substituted as previously defined. Even more particularly, Cy³ is a 3- to 7-membered heterocyclic monocyclic ring, and is optionally substituted with one or more substituents Z³ optionally substituted as previously defined. Even more particularly, Z³ in R^{e} is (C₁-C₆)alkyl substituted with one or more substituents R^{a}.

In another embodiment, optionally in combination with one or more features of the various embodiments described above or below, the invention relates to a compound of formula (I) as described in the first or second aspect, wherein R₃ is —NR^{b′}R^{d}, and R^{d} is Cy¹ optionally substituted with one or more substituents Z¹ optionally substituted as previously defined. More particularly, Cy¹ in R^{d} is a known ring system selected from group consisting of a 3- to 7-membered saturated or partially unsaturated carbocyclic or heterocyclic monocyclic ring; and 3- to 7-membered saturated or partially unsaturated carbocyclic or heterocyclic monocyclic ring, which is fused, bridged-fused or spiro-fused, more particularly bridged-fused or spiro-fused, to a 3- to 7-membered saturated or partially unsaturated carbocyclic or heterocyclic monocyclic ring, wherein Cy¹ is optionally substituted as previously defined. Even more particularly, Z¹ in R^{d} is (C₁-C₆)alkyl substituted with one or more substituents R^{a}.

In another embodiment, optionally in combination with one or more features of the various embodiments described above or below, the invention relates to a compound of formula (I) as previously described, wherein R₃ is a moiety of formula (XIV):

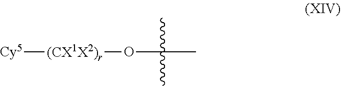

wherein

Cy⁵ is a 3- to 7-membered saturated or partially unsaturated or aromatic carbocyclic or heterocyclic monocyclic ring or a 3- to 7-membered saturated or partially unsaturated or aromatic carbocyclic or heterocyclic monocyclic ring, which is fused, bridged-fused or spiro-fused to a 3- to 7-membered saturated or partially unsaturated or aromatic carbocyclic or heterocyclic monocyclic ring, and Cy⁵ is optionally substituted with one or more substituents selected from halogen and (C₁-C₃)alkyl optionally substituted with one or more halogen atoms,

X¹ and X² are independently H or halogen, and

r is a value selected from 0 to 6.

More particularly, R₃ is a moiety of formula (XIV) wherein Cy⁵ is a 3- to 7-membered saturated or partially unsaturated carbocyclic or heterocyclic monocyclic ring or a 3- to 7-membered saturated or partially unsaturated carbocyclic or heterocyclic monocyclic ring, which is fused, bridged-fused or spiro-fused to a 3- to 7-membered saturated or partially unsaturated carbocyclic or heterocyclic monocyclic ring, and Cy₅ is optionally substituted with one or more substituents selected from halogen and (C₁-C₃)alkyl optionally substituted with one or more halogen atoms, X¹ and X² are independently H or halogen, and r is a value selected from 0 to 6.

More particularly, R₃ is a moiety of formula (XIV) wherein Cy⁵ is a 3- to 7-membered saturated heterocyclic monocyclic ring or a 3- to 7-membered saturated carbocyclic or heterocyclic monocyclic ring, which is spiro-fused to a 3- to 7-membered saturated carbocyclic or heterocyclic monocyclic ring, and Cy⁵ is optionally substituted as previously defined, X¹ and X² are H, and r is a value selected from 0 to 6.

In another embodiment, optionally in combination with one or more features of the various embodiments described above or below, the invention relates to a compound of formula (I) as previously described, wherein R₃ is selected from the group consisting of the following moieties:

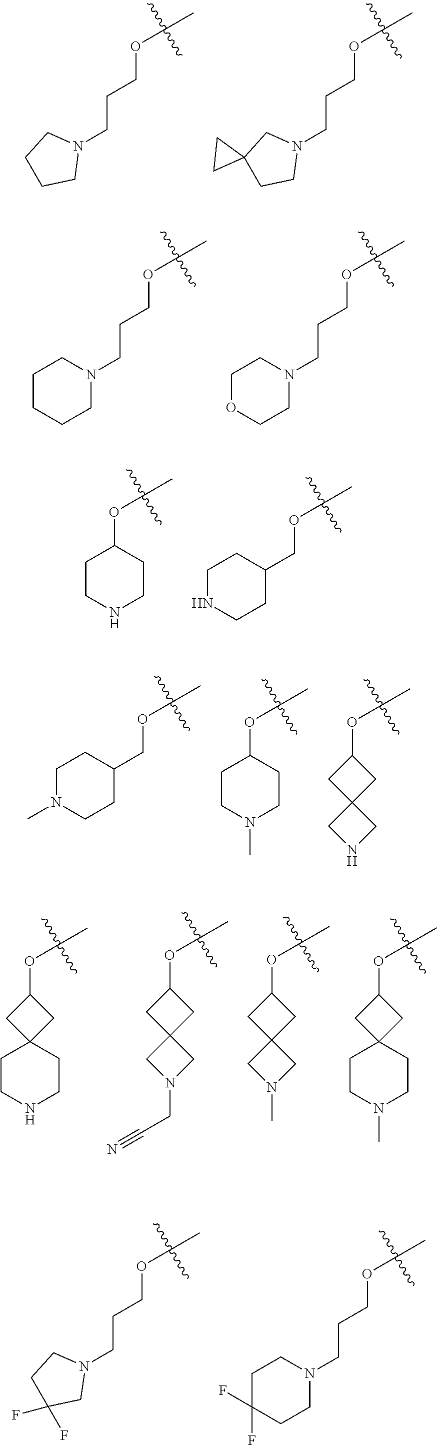

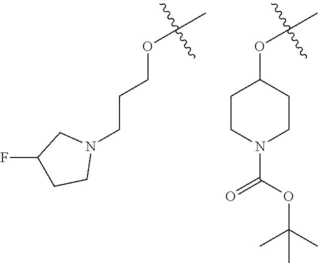 [SMILES: *OCCCN1CCC(F)C1 ; *OC1CCN(C(=O)OC(C)(C)C)CC1]

In another embodiment, optionally in combination with one or more features of the various embodiments described above or below, the invention relates to a compound of formula (I) as previously described, wherein R is a radical of formula (C) (i.e. a compound (IC)):

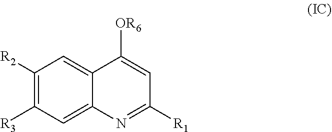

and R₆ is Cy¹ optionally substituted with one or more substituents Z² optionally substituted as previously defined. Even more particularly, Cy¹ in R₆ is a known ring system selected from group consisting of 3- to 7-membered saturated or partially unsaturated carbocyclic or heterocyclic monocyclic ring; and 3- to 7-membered saturated or partially unsaturated carbocyclic or heterocyclic monocyclic ring, which is fused, bridged-fused or spiro-fused, more particularly bridged-fused or spiro-fused, to a 3- to 7-membered saturated or partially unsaturated carbocyclic or heterocyclic monocyclic ring, wherein Cy¹ is optionally substituted as previously defined. Even more particularly, Z² is (C₁-C₆)alkyl optionally substituted with one or more substituents R^{a}.

In another embodiment, optionally in combination with one or more features of the various embodiments described above or below, the invention relates to a compound of formula (I) as previously described, wherein R is a radical of formula (C) (i.e. a compound (IC)), and R₆ is Z¹; more particularly, Z¹ is (C₁-C₆)alkyl, optionally substituted as previously defined. Even more particularly, Z¹ is substituted with Cy³, wherein Cy³ is optionally substituted as previously defined. Even more particularly, Cy³ is optionally substituted with one or more substituents Z³ optionally substituted as previously defined. Even more particularly, Cy³ in R₆ is a known ring system selected from group consisting of phenyl; 5- or 6-membered heteroaromatic ring; 3- to 7-membered saturated or partially unsaturated carbocyclic or heterocyclic monocyclic ring; and 3- to 7-membered saturated or partially unsaturated carbocyclic or heterocyclic monocyclic ring, which is fused, bridged-fused or spiro-fused to a 3- to 7-membered saturated or partially unsaturated carbocyclic or heterocyclic monocyclic ring, wherein Cy³ is optionally substituted as previously defined. Even more particularly, Cy³ in R₆ is selected from 3- to 7-membered saturated or partially unsaturated carbocyclic or heterocyclic monocyclic ring; and 3- to 7-membered saturated or partially unsaturated carbocyclic or heterocyclic monocyclic ring, which is fused, bridged-fused or spiro-fused, more particularly bridged-fused or spiro-fused, to a 3- to 7-membered saturated or partially unsaturated carbocyclic or heterocyclic monocyclic ring, wherein Cy³ is optionally substituted as previously defined. Even more particularly, Z³ in R₆ is (C₁-C₆)alkyl optionally substituted with one or more substituents R^{a}.

In another embodiment, optionally in combination with one or more features of the various embodiments described above or below, the invention relates to a compound of formula (I) as previously described, wherein R is a radical of formula (C) (i.e. a compound (IC)) selected from the group consisting of the following moieties:

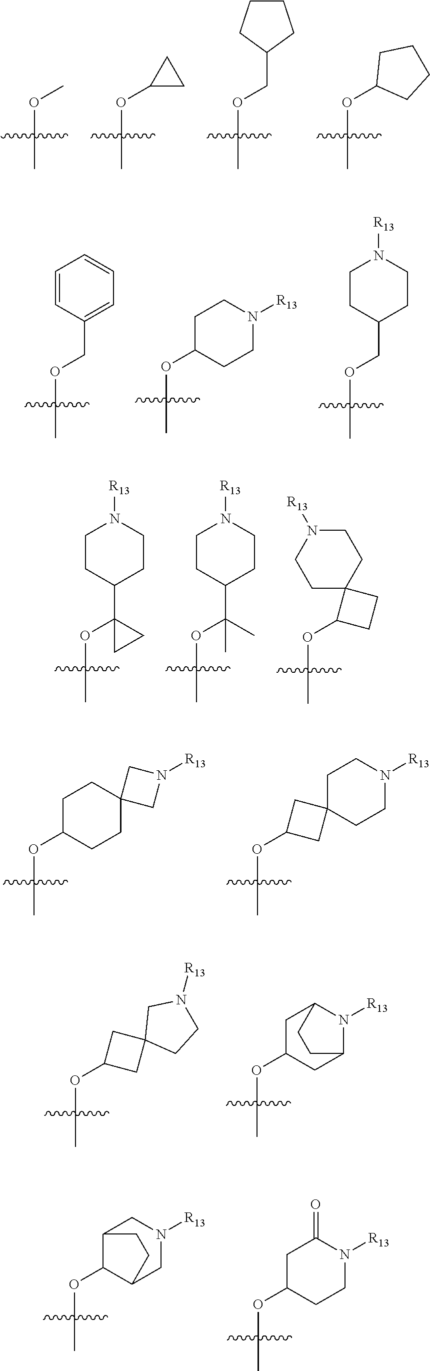

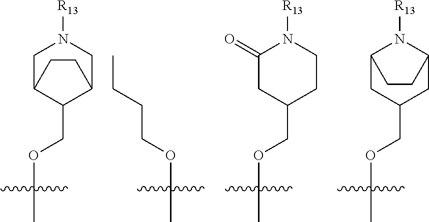

wherein R₁₃ is selected from the group consisting of H, methyl, isopropyl and cyclopropyl.

In another embodiment, optionally in combination with one or more features of the various embodiments described above or below, the invention relates to a compound of formula (I) as previously described, wherein R is a radical of formula (D) (i.e. a compound (ID)):

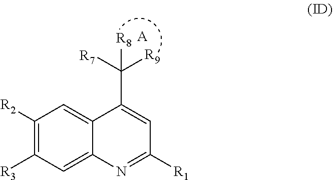

the dotted line means the absence of a ring system A; R₇ is Cy¹ optionally substituted with one or more substituents Z² optionally substituted as previously defined; and R₈ and R₉ are independently selected from the group consisting of H, halogen, (C₁-C₆)alkyl optionally substituted with one or more halogen atoms, and a 3- to 7-membered saturated or partially unsaturated carbocyclic monocyclic ring optionally substituted with one or more halogen atoms. Even more particularly, Cy¹ in R₇ is a known ring system selected from group consisting of 3- to 7-membered saturated or partially unsaturated carbocyclic or heterocyclic monocyclic ring; and 3- to 7-membered saturated or partially unsaturated carbocyclic or heterocyclic monocyclic ring, which is fused, bridged-fused or spiro-fused, more particularly bridged-fused or spiro-fused, to a 3- to 7-membered saturated or partially unsaturated carbocyclic or heterocyclic monocyclic ring, wherein Cy¹ is optionally substituted as previously defined. Even more particularly, Cy¹ in R₇ is 3- to 7-membered saturated or partially unsaturated heterocyclic monocyclic ring, wherein Cy¹ is optionally substituted as previously defined. Even more particularly, Z² in R⁷ is (C₁-C₆)alkyl optionally substituted with one or more substituents R^{a}. Even more particularly, R₈ and R₉ are independently selected from the group consisting of H, halogen, and (C₁-C₆)alkyl optionally substituted with one or more halogen atoms. Even more particularly, R₈ and R₉ are independently H or halogen. Even more particularly, R₈ and R₉ are independently H or F.

In another embodiment, optionally in combination with one or more features of the various embodiments described above or below, the invention relates to a compound of formula (I) as previously described, wherein R is a radical of formula (D) (i.e. a compound (ID)), the dotted line means the absence of a ring system A; R₇ is Z¹; more particularly, Z¹ is (C₁-C₆)alkyl, optionally substituted as previously defined; and R₈ and R₉ are independently selected from the group consisting of H, halogen, (C₁-C₆)alkyl optionally substituted with one or more halogen atoms, and a 3- to 7-membered saturated or partially unsaturated carbocyclic monocyclic ring optionally substituted with one or more halogen atoms. Even more particularly, Z¹ in R₇ is substituted with Cy³, wherein Cy³ is optionally substituted as previously defined. Even more particularly, Cy³ is optionally substituted with one or more substituents Z³ optionally substituted as previously defined. Even more particularly, Cy³ in R₇ is a known ring system selected from group consisting of phenyl; 5- or 6-membered heteroaromatic ring; 3- to 7-membered saturated or partially unsaturated carbocyclic or heterocyclic monocyclic ring; and 3- to 7-membered saturated or partially unsaturated carbocyclic or heterocyclic monocyclic ring, which is fused, bridged-fused or spiro-fused to a 3- to 7-membered saturated or partially unsaturated carbocyclic or heterocyclic monocyclic ring, wherein Cy³ is optionally substituted as previously defined.

Even more particularly, Cy³ in R₇ is selected from 3- to 7-membered saturated or partially unsaturated carbocyclic or heterocyclic monocyclic ring; and 3- to 7-membered saturated or partially unsaturated carbocyclic or heterocyclic monocyclic ring, which is fused, bridged-fused or spiro-fused, more particularly bridged-fused or spiro-fused, to a 3- to 7-membered saturated or partially unsaturated carbocyclic or heterocyclic monocyclic ring, wherein Cy³ is optionally substituted as previously defined. Even more particularly, Z³ in R₇ is (C₁-C₆)alkyl optionally substituted with one or more substituents R^{a}. Even more particularly, Z³ in R₇ is (C₁-C₆)alkyl optionally substituted with one or more substituents R^{a}. Even more particularly, R₈ and R₉ are independently selected from the group consisting of H, halogen, and (C₁-C₆)alkyl optionally substituted with one or more halogen atoms. Even more particularly, R₈ and R₉ are independently H or halogen. Even more particularly, R₈ and R₉ are independently H or F. Even more particularly, R₈ and R₉ are H.

In another embodiment, optionally in combination with one or more features of the various embodiments described above or below, the invention relates to a compound of formula (I) as previously described, wherein R is a radical of formula (D) (i.e. a compound (ID)); the dotted line means the presence of a ring system A; R⁷ is absent; and R₈ and R₉, together with the carbon atom to which they are attached, form a known ring system A comprising a 3- to 7-membered partially unsaturated carbocyclic or heterocyclic monocyclic ring, more particularly a 3- to 7-membered heterocyclic monocyclic ring, wherein the ring A is saturated or it contains at least one unsaturation between the carbon atom to which R₈ and R₉ are attached and the contiguous carbon atom; and the ring system is optionally substituted with one or more substituents R^{a}, and/or one or more substituents Z¹ optionally substituted as previously defined. Even more particularly, Z¹ is (C₁-C₆)alkyl optionally substituted with one or more substituents R^{a}.

In another embodiment, optionally in combination with one or more features of the various embodiments described above or below, the invention relates to a compound of formula (I) as previously described, wherein R is a radical of formula (D) (i.e. a compound (ID)); the dotted line means the presence of a ring system A; R₇ is Cy¹ optionally substituted with one or more substituents Z² optionally substituted as previously defined; and R₈ and R₉, together with the carbon atom to which they are attached form a known ring system A comprising a 3- to 7-membered saturated or partially unsaturated carbocyclic monocyclic ring optionally substituted as previously defined, more particularly substituted with one or more substituents Z¹, wherein Z¹ in ring A is optionally substituted as previously defined. Even more particularly, Cy¹ in R₇ is a known ring system selected from group consisting of 3- to 7-membered saturated or partially unsaturated carbocyclic or heterocyclic monocyclic ring; and 3- to 7-membered saturated or partially unsaturated carbocyclic or heterocyclic monocyclic ring, which is fused, bridged-fused or spiro-fused, more particularly bridged-fused or spiro-fused, to a 3- to 7-membered saturated or partially unsaturated carbocyclic or heterocyclic monocyclic ring, wherein Cy¹ is optionally substituted as previously defined. Even more particularly, Cy¹ in R₇ is 3- to 7-membered saturated or partially unsaturated heterocyclic monocyclic ring, wherein Cy¹ is optionally substituted as previously defined. Even more particularly, Z² in R⁷ is (C₁-C₆)alkyl optionally substituted with one or more substituents R^{a}.

In another embodiment, optionally in combination with one or more features of the various embodiments described above or below, the invention relates to a compound of formula (I) as previously described, wherein R is a radical of formula (D) (i.e. a compound (ID)), the dotted line means the presence of a ring system A; R₇ is Z¹; more particularly, Z¹ is (C₁-C₆)alkyl, optionally substituted as previously defined; and R₈ and R₉, together with the carbon atom to which they are attached form a known ring system A comprising a 3- to 7-membered saturated or partially unsaturated carbocyclic monocyclic ring optionally substituted as previously defined, more particularly substituted with one or more substituents Z¹, wherein Z¹ in ring A is optionally substituted as previously defined. Even more particularly, Z¹ in R₇ is substituted with Cy³, wherein Cy³ is optionally substituted as previously defined. Even more particularly, Cy³ is optionally substituted with one or more substituents Z³ optionally substituted as previously defined. Even more particularly, Cy³ in R₇ is a known ring system selected from group consisting of phenyl; 5- or 6-membered heteroaromatic ring; 3- to 7-membered saturated or partially unsaturated carbocyclic or heterocyclic monocyclic ring; and 3- to 7-membered saturated or partially unsaturated carbocyclic or heterocyclic monocyclic ring, which is fused, bridged-fused or spiro-fused to a 3- to 7-membered saturated or partially unsaturated carbocyclic or heterocyclic monocyclic ring, wherein Cy³ is optionally substituted as previously defined. Even more particularly, Cy³ in R₇ is selected from 3- to 7-membered saturated or partially unsaturated carbocyclic or heterocyclic monocyclic ring; and 3- to 7-membered saturated or partially unsaturated carbocyclic or heterocyclic monocyclic ring, which is fused, bridged-fused or spiro-fused, more particularly bridged-fused or spiro-fused, to a 3- to 7-membered saturated or partially unsaturated carbocyclic or heterocyclic monocyclic ring, wherein Cy³ is optionally substituted as previously defined. Even more particularly, Z³ in R₇ is (C₁-C₆)alkyl optionally substituted with one or more substituents R^{a}. Even more particularly, Z³ in R₇ is (C₁-C₆)alkyl optionally substituted with one or more substituents R^{a}.

In another embodiment, optionally in combination with one or more features of the various embodiments described above or below, the invention relates to a compound of formula (I) as previously described, wherein R is a radical of formula (D), and the dotted line means the presence of a ring system A; R₇ is selected from the group consisting of H, R^{a}, and Z¹ optionally substituted with one or more substituents R^{a}; and R₈ and R₉, together with the carbon atom to which they are attached form a known ring system A as previously defined. More particularly, Z¹ in R₇ is (C₁-C₃)alkyl optionally substituted with one or more substituents R^{a}, and R^{a} in R₇ is selected from halogen, —OR^{b′} (in particular wherein R^{b} is H or (C₁-C₃)alkyl), and —CN. Even more particularly, R₈ and R₉, together with the carbon atom to which they are attached form a known ring system A comprising a 3- to 7-membered saturated or partially unsaturated carbocyclic or heterocyclic monocyclic ring optionally substituted with one or more substituents Z¹, wherein Z¹ in ring A is optionally substituted as previously defined, more particularly Z¹ in ring A is (C₁-C₆)alkyl optionally substituted with one or more substituents R^{a}.

In another embodiment, optionally in combination with one or more features of the various embodiments described above or below, the invention relates to a compound of formula (I) as previously described, wherein R is a radical of formula (D) (i.e. a compound (ID)) selected from the group consisting of the following moieties:

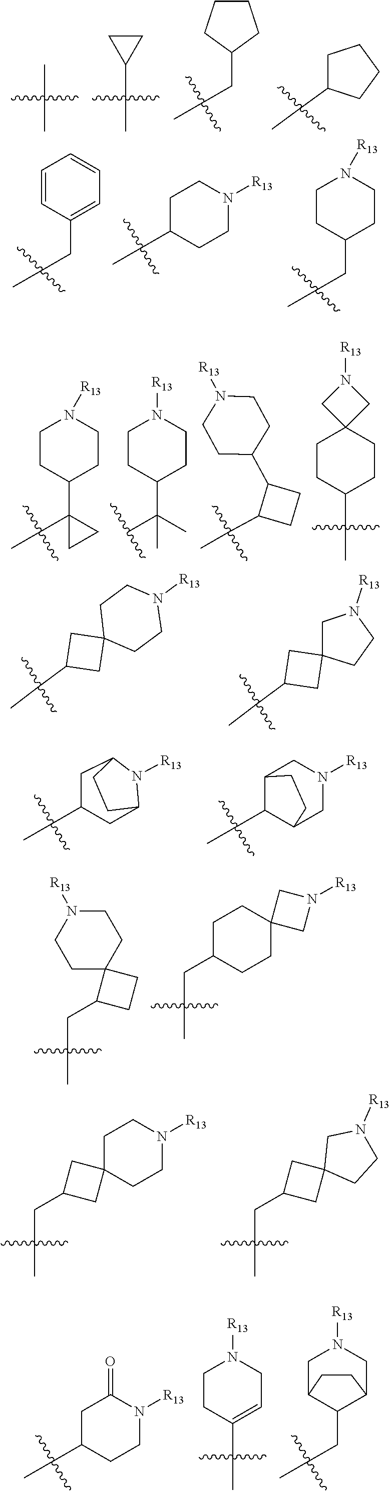

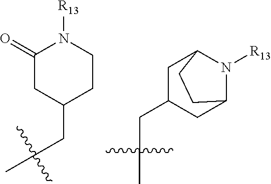

wherein R₁₃ is selected from the group consisting of H, methyl, isopropyl and cyclopropyl.

In another embodiment, optionally in combination with one or more features of the various embodiments described above or below, the invention relates to a compound of formula (I) as previously described, wherein R is a radical of formula (E) (i.e. a compound (IE)):

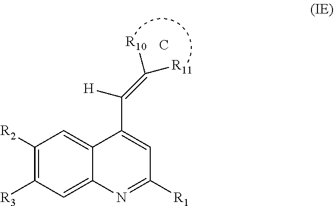

the dotted line means the presence of a ring system C; R₁₀ and R₁₁, together with the carbon atom to which they are attached, form a known ring system C comprising a 3- to 7-membered saturated or partially unsaturated carbocyclic or heterocyclic monocyclic ring, which is optionally fused, bridged-fused or spiro-fused, more particularly bridged-fused or spiro-fused, to a 3- to 7-membered saturated or partially unsaturated or aromatic carbocyclic or heterocyclic monocyclic ring; wherein the ring system C is optionally substituted as previously defined. More particularly, R₁₀ and R₁₁, together with the carbon atom to which they are attached, form a known ring system C comprising a 3- to 7-membered saturated or partially unsaturated carbocyclic or heterocyclic monocyclic ring, wherein the ring system C is optionally substituted as previously defined. Even more particularly, ring C is optionally substituted with one or more substituents R^{a} and/or Z¹, more particularly Z¹ is (C₁-C₆)alkyl, optionally substituted with one or more substituents R^{a}.

In another embodiment, optionally in combination with one or more features of the various embodiments described above or below, the invention relates to a compound of formula (I) as previously described, wherein R is a radical of formula (E) (i.e. a compound (IE)) selected from the group consisting of the following moieties:

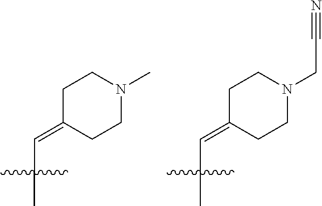

The embodiments defined above apply to all the compounds of formula (I), i.e., defined in any of the aspects and embodiments of the invention, when possible.

In another embodiment of the invention, in the compound of formula (I) R is a radical of formula (A), and the compound of formula (I) is selected from the group consisting of:

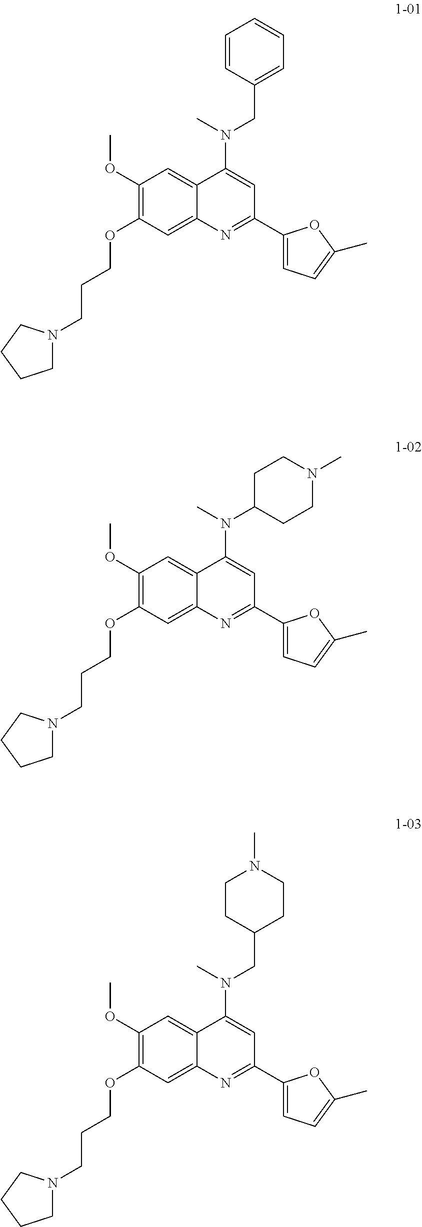

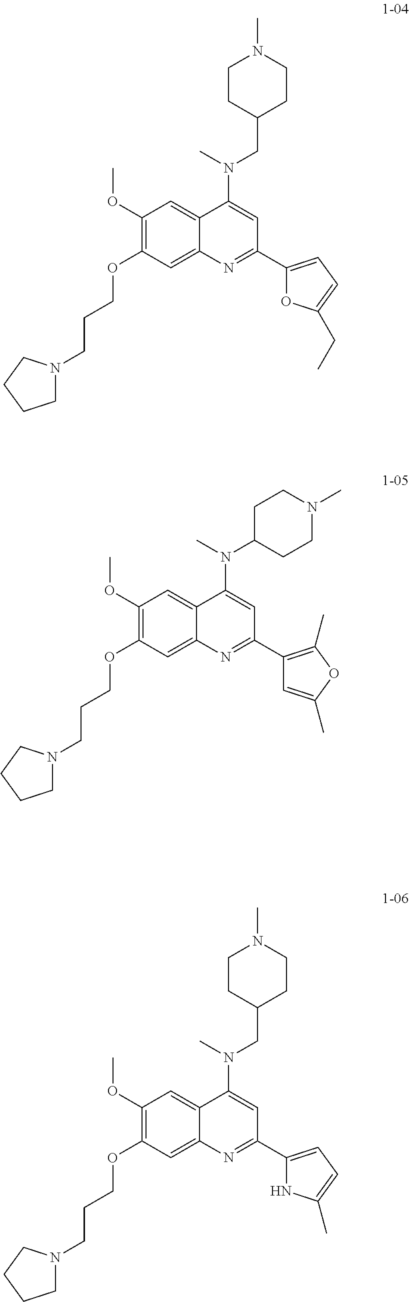

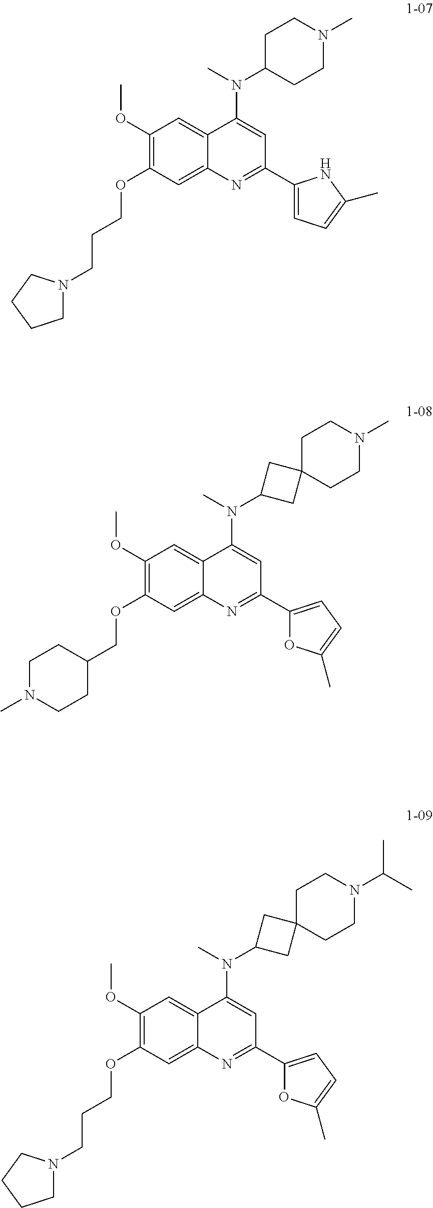

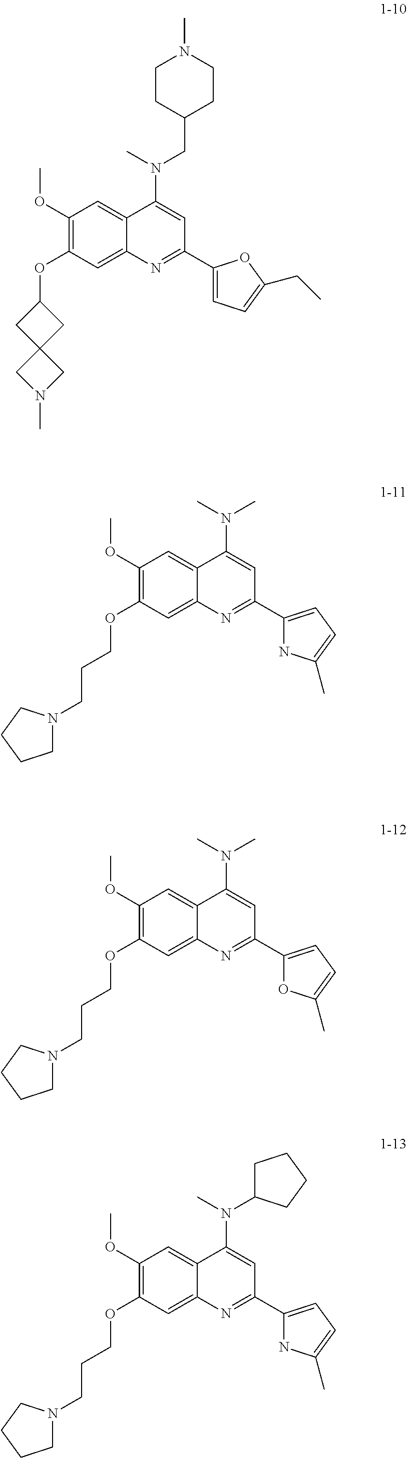

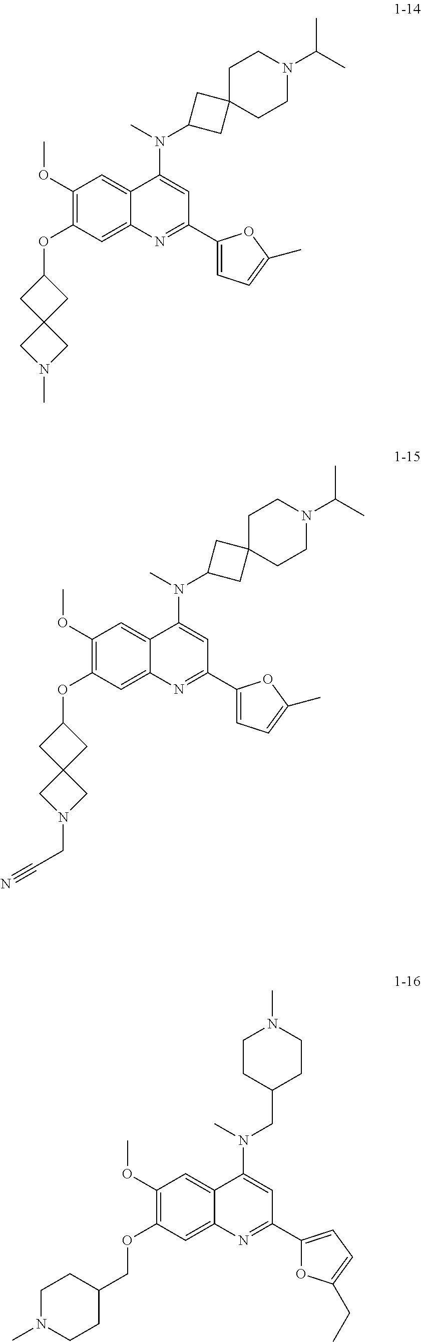

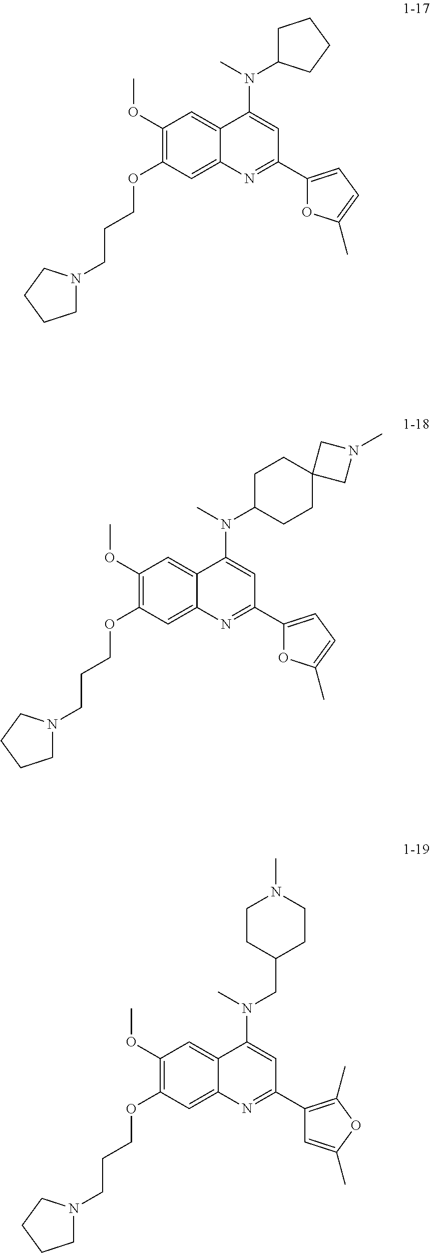

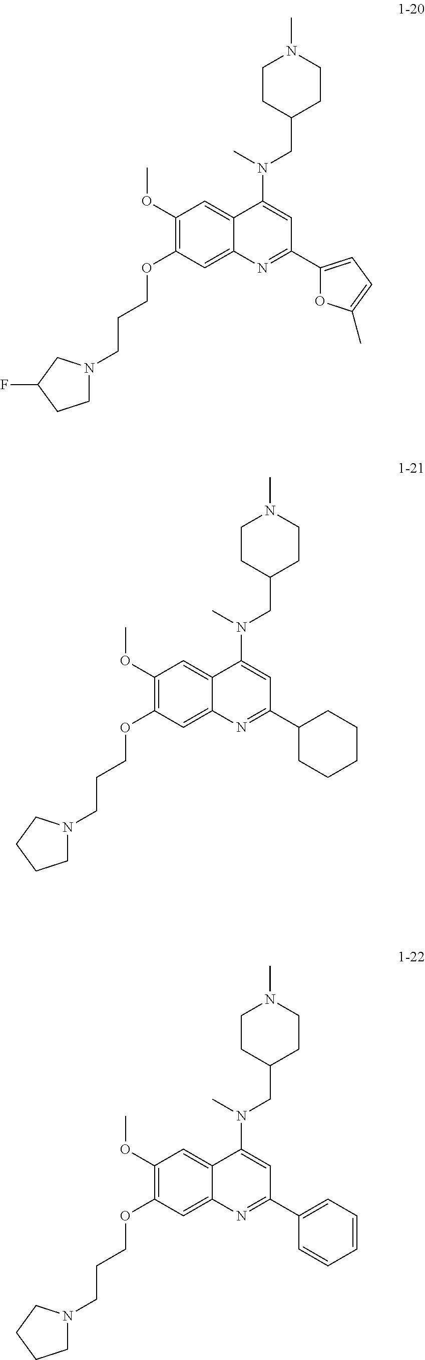

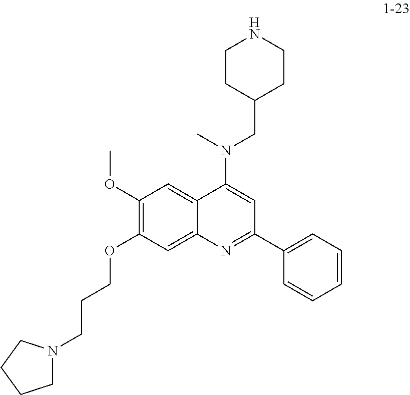

In another embodiment of the invention, in the compound of formula (I) R is a radical of formula (B), and the compound of formula (I) is selected from the group consisting of:

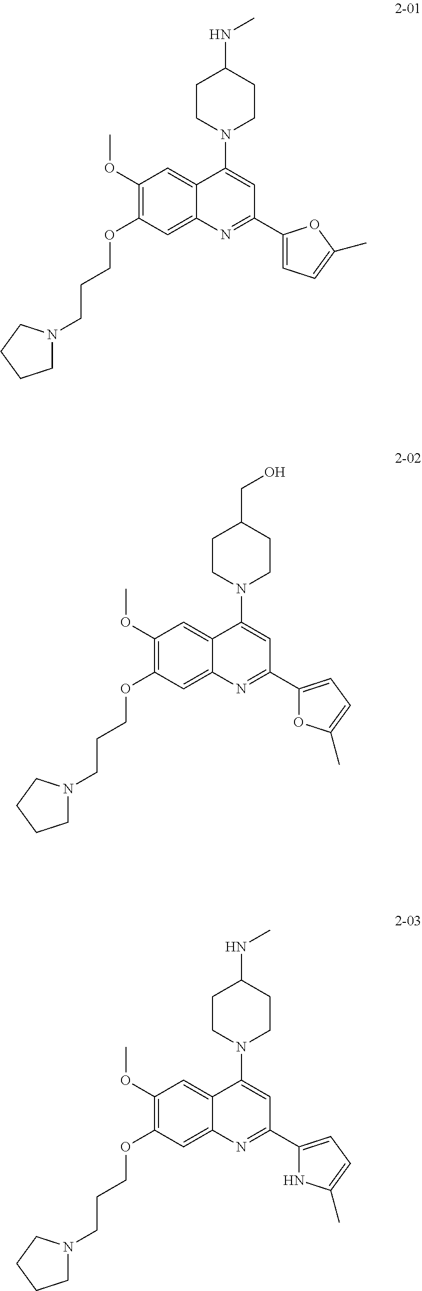

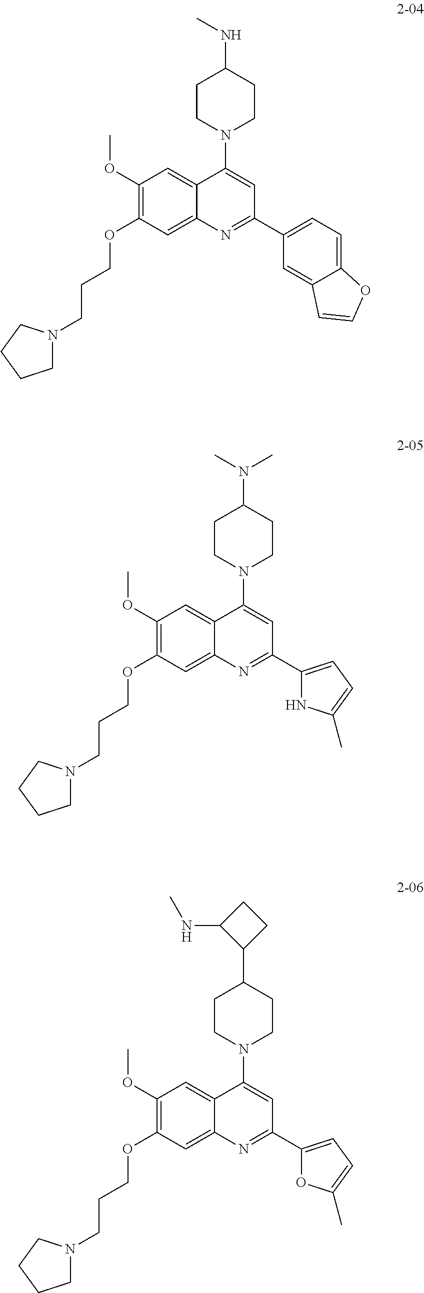

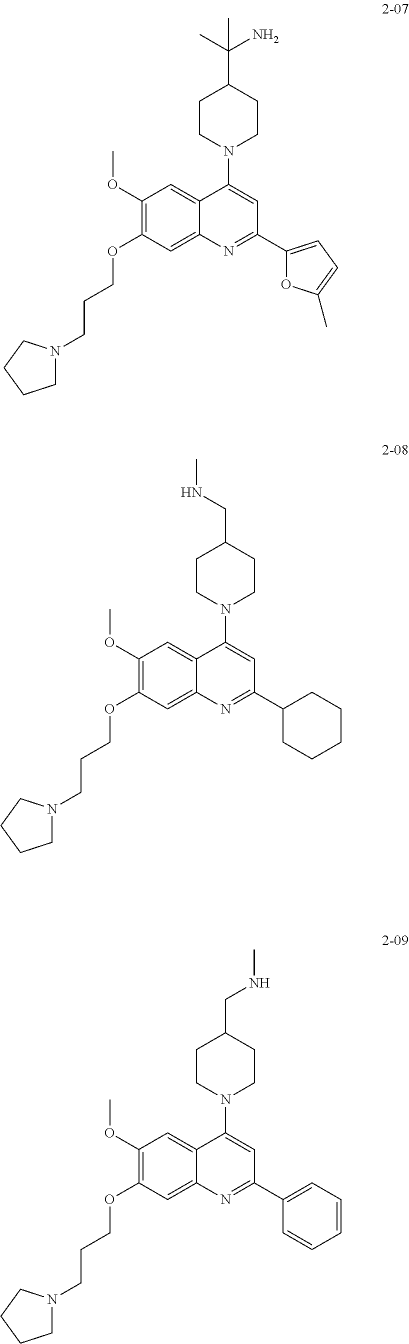

In another embodiment of the invention, in the compound of formula (I) R is a radical of formula (C), and the compound of formula (I) is selected from the group consisting of:

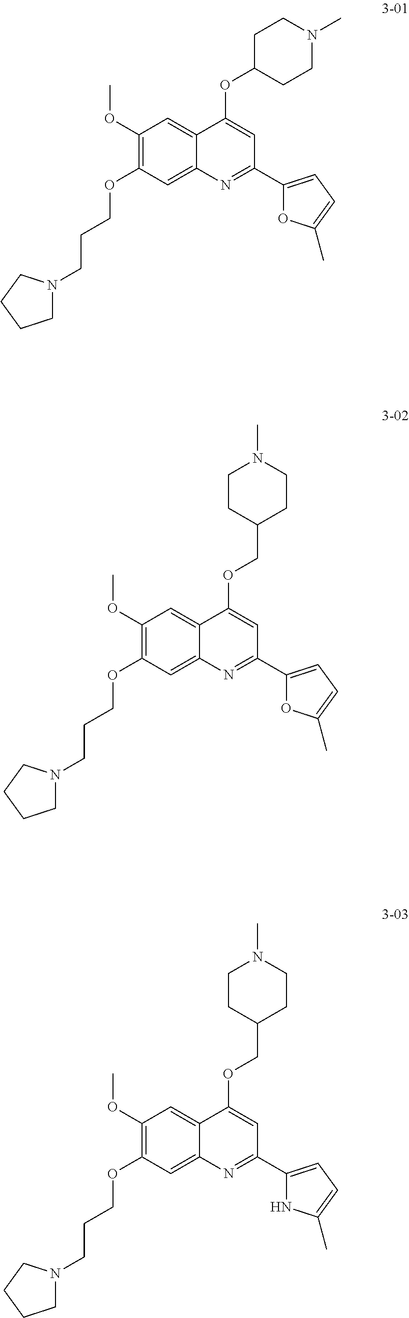

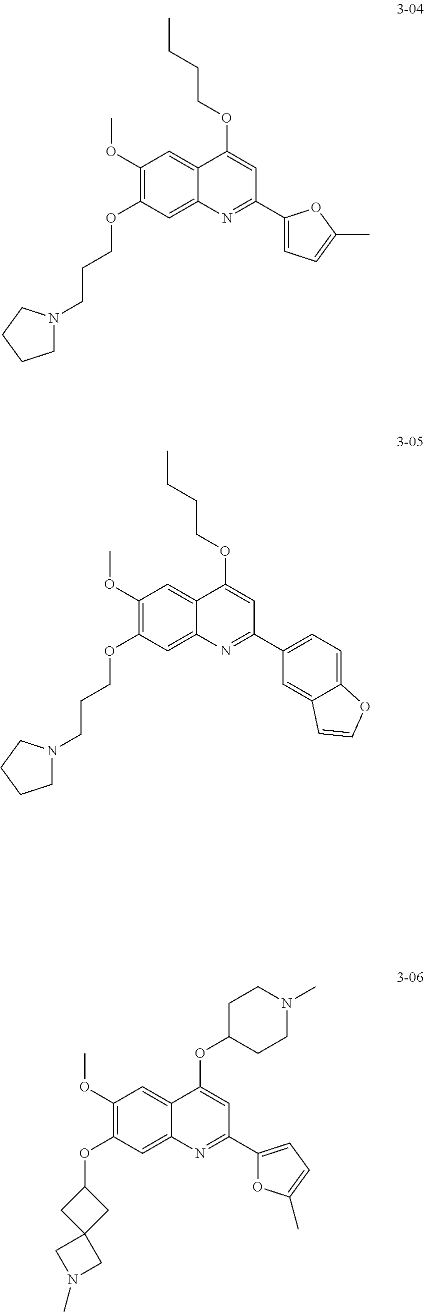

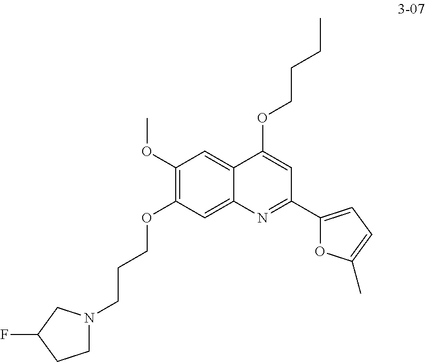

In another embodiment of the invention, in the compound of formula (I) R is a radical of formula (D), and the compound of formula (I) is selected from the group consisting of:

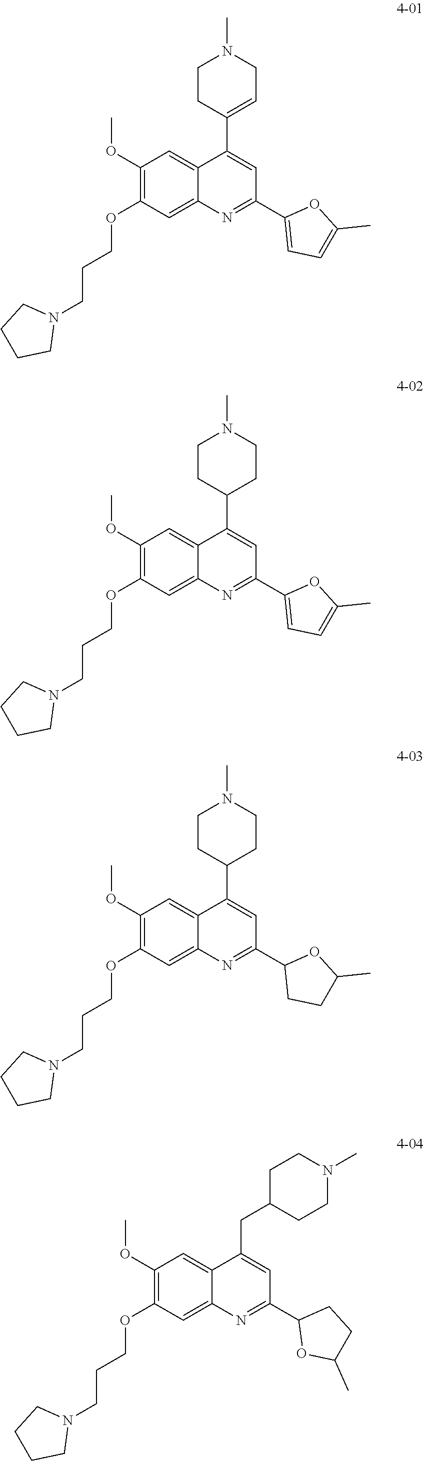

In another embodiment of the invention, in the compound of formula (I) R is a radical of formula (E), and the compound of formula (I) is selected from the group consisting of:

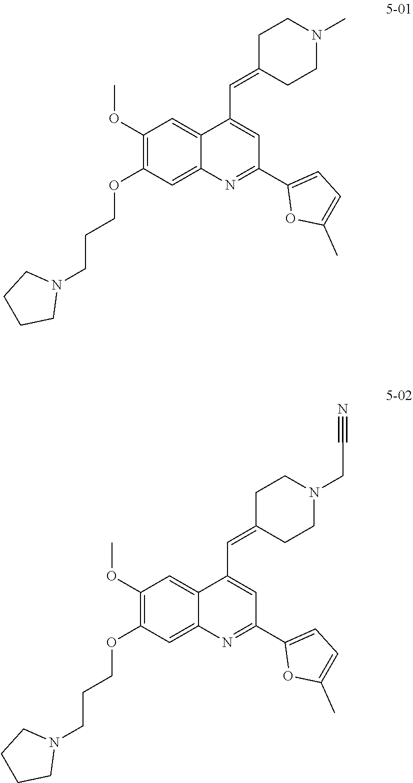

Processes for the preparation of compounds of formula (I) are also part of the invention as well as intermediates used in these processes.

Processes for the preparation of compounds of formula (I) are also part of the invention as well as intermediates used in these processes.

For example, a compound of formula (I) which is a compound of formula (IA) or a compound formula (IB) can be obtained from a compound of formula (II) by reacting them with a compound of formula (III) or a compound of formula (IV), respectively, as shown in the scheme below:

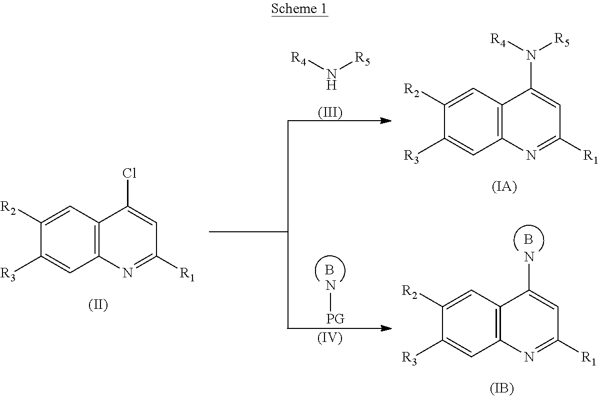

wherein R₁-R₅ and ring B are as previously defined, and PG is an amino protective group, such as a tert-butoxycarbonyl (BOC).

In both cases the reactions for obtaining a compound of formula (IA) or a compound of formula (IB) are carried out optionally in the presence of p-toluenesulfonic acid (PTSA), in a suitable solvent, such as tert-butanol at a suitable temperature, preferably heating at a temperature around 100-120 OC.

A compound of formula (I) which is a compound of formula (IC) can be obtained from a compound of formula (II), which is firstly converted into a compound of formula (V) and then subsequently reacted with a compound of formula (VI) as shown in the scheme below:

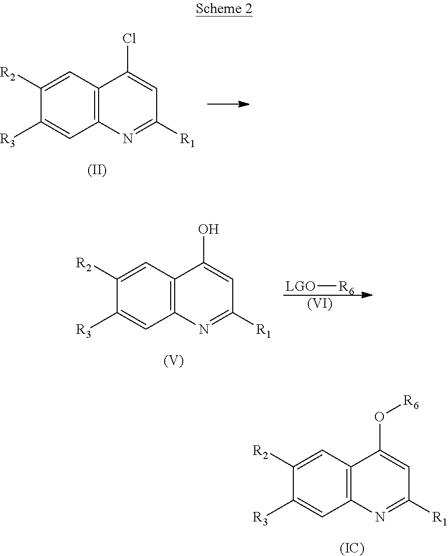

wherein R₁-R₃ and R₆ are as previously defined, and LG is a leaving group, such as a methanesulfonate (Ms).

The first conversion is carried out with a boronic derivative such as 4,4,5,5-tetramethyl-2-(4,4,5,5-tetramethyl-1,3,2-dioxaborolan-2-yl)-1,3,2-dioxaborolane, in the presence of a palladium catalyst, such as e.g. [1,1′-Bis(diphenylphosphino)ferrocene]palladium(II) dichloride (Pd(dppf)Cl₂) and KOAc, in a suitable solvent, such as e.g. dioxane, at a suitable temperature, preferably heating at a temperature around 100-120° C.; and then by reacting the intermediate obtained with hydrogen peroxide in a suitable solvent, such as dichloromethane.

The second conversion is carried out in the presence of a base, such as Cs₂CO₃, in a suitable solvent such as N,N-dimethylformamide (DMF) preferably heating at a temperature around 80-120° C.; and then the intermediate obtained is reacted with an acid such as HCl/EtOAc or HCl/MeOH. Finally the intermediate obtained is reacted with an aldehyde, such as e.g. (HCHO)ₙ in the presence of a reducing agent, such as NaBH(OAc)₃ or NaBH₃CN, in the presence of HCOOH in a suitable solvent, such as methanol, at a suitable temperature, preferably heating at 40-70° C.

Alternatively, a compound of formula (II) can be directly converted into a compound of formula (IC) by reaction with a compound of formula (VI′) HO—R₆, optionally in the presence of p-toluenesulfonic acid (PTSA) at a suitable temperature, preferably heating at a temperature around 100-120 OC.

A compound of formula (I) which is a compound of formula (ID), wherein the dotted line means the presence of a ring system A; R⁷ is absent; and R₈ and R₉, together with the carbon atom to which they are attached, form a known ring system A containing at least one unsaturation between the carbon atom to which R₈ and R₉ are attached and the contiguous carbon atom (i.e. a compound of formula (ID′)) can be obtained by reacting a compound of formula (II) with a boronic derivative of formula (VII), as shown in the scheme below:

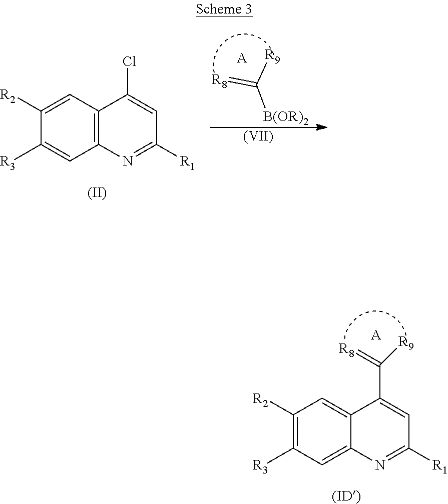

wherein R₁-R₃ and, R₈-R₉ and ring A are as previously defined, and each R is H, (C₁-C₆)alkyl or, alternatively, two R groups together with the B atom to which they are attached may form a cycle. This conversion is carried out in the presence of a palladium catalyst, such as e.g.

Tetrakis(triphenylphosphine)-palladium(0) (Pd(PPh₃)₄) and KOAc or K₂CO₃, in a suitable solvent, such as e.g. dioxane optionally mixed with water, at a suitable temperature, preferably heating at a temperature around 100-120° C.

A compound of formula (I) which is a compound of formula (IE) can be obtained by reacting a compound of formula (II) with a boronic derivative of formula (VIII), as shown in the scheme below:

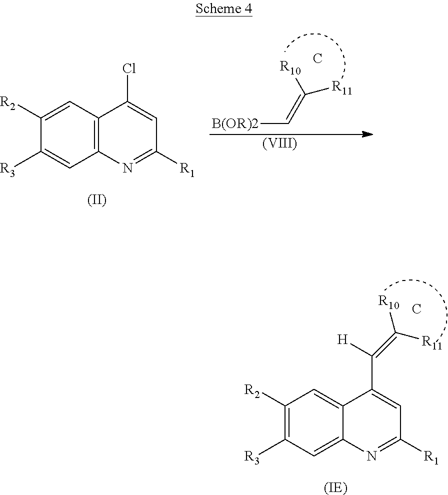

wherein R₁-R₃ and, R₁₀-R₁₁ and ring C are as previously defined, and each R is H, (C₁-C₆)alkyl or, alternatively, two R groups together with the B atom to which they are attached may form a cycle.

This conversion is carried out in the same conditions as previously described for the conversion of a compound of formula (II) into a compound of formula (ID′).

Compound (IE) may be converted into a compound of formula (ID), wherein the dotted line means the absence of a ring system A. This conversion is carried out by hydrogenation, e.g. in the presence of Pd/C in a suitable solvent such as methanol, at a suitable temperature, preferably, room temperature.

A compound of formula (II) can be obtained from a quinoline of formula (XII) which is firstly converted into a compound of formula (XI). This compound is then reacted with a compound of formula (X) to give a compound of formula (IX) which is converted into a compound (II).

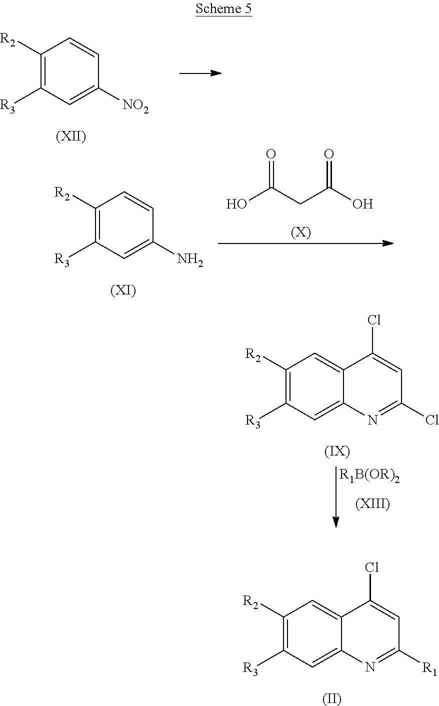

wherein R₁-R₃ are as previously defined, and each R is H, (C₁-C₆)alkyl or, alternatively, two R groups together with the B atom to which they are attached may form a cycle.

The reduction of the compound of formula (XII) into a compound of formula (XI) is carried out by hydrogenation, e.g. in the presence of Pd/C in a suitable solvent such as methanol, whereas the conversion of a compound of formula (XI) into a compound of formula (IX) is carried out in the presence of a halogenating agent, such as e.g. POCl₃, at a suitable temperature, preferably heating.

The conversion of a compound of formula (IX) into a compound of formula (II) is carried out in the presence of a palladium catalyst, such as e.g. Tetrakis(triphenylphosphine)palladium(0) (Pd(PPh₃)₄) and a base, such as e.g. K₂CO₃ or Na₂CO₃, in a suitable solvent, such as e.g. dioxane optionally mixed with water, at a suitable temperature, preferably heating, particularly at about 100-120° C.

Alternatively, the reactions described above can be carried out in a different order. Thus, for example the above described reactions carried out on intermediates already containing substituents R₁-R₃ can also be performed on analogue intermediates containing one or more precursors of substituents R₁-R₃, which are subsequently transformed into groups R₁-R₃.

Compounds of formula (I) may also be converted into other compounds of formula (I) by reaction well known in the art. The compounds of formulas (III), (IV), (VI), (VII), (VIII), (X), and (XIII) are commercially available or can be obtained by conventional synthetic processes.

The present invention also relates to a pharmaceutical or veterinary composition comprising an effective amount of a compound of formula (I) as defined above, or a pharmaceutically or veterinary acceptable salt thereof, or any stereoisomer either of the compound of formula (I) or of their pharmaceutically or veterinary acceptable salts, together with pharmaceutically or veterinary acceptable excipients or carriers.

The expression “therapeutically effective amount” as used herein, refers to the amount of a compound that, when administered, is sufficient to prevent development of, or alleviate to some extent, one or more of the symptoms of the disease which is addressed. The specific dose of the compound of the invention to obtain a therapeutic benefit may vary depending on the particular circumstances of the individual patient including, among others, the size, weight, age and sex of the patient, the nature and stage of the disease, the aggressiveness of the disease, and the route of administration. For example, a dose of from about 0.01 to about 300 mg/kg may be used.

The expression “pharmaceutically or veterinary acceptable excipients or carriers” refers to pharmaceutically or veterinary acceptable materials, compositions or vehicles. Each component must be pharmaceutically or veterinary acceptable in the sense of being compatible with the other ingredients of the pharmaceutical or veterinary composition. It must also be suitable for use in contact with the tissue or organ of humans and animals without excessive toxicity, irritation, allergic response, immunogenicity or other problems or complications commensurate with a reasonable benefit/risk ratio.

The election of the pharmaceutical or veterinary formulation will depend upon the nature of the active compound and its route of administration. Any route of administration may be used, for example oral, parenteral and topical administration.

For example, the pharmaceutical or veterinary composition may be formulated for oral administration and may contain one or more physiologically compatible carriers or excipients, in solid or liquid form. These preparations may contain conventional ingredients such as binding agents, fillers, lubricants, and acceptable wetting agents.

The pharmaceutical or veterinary composition may be formulated for parenteral administration in combination with conventional injectable liquid carriers, such as water or suitable alcohols. Conventional pharmaceutical or veterinary excipients for injection, such as stabilizing agents, solubilizing agents, and buffers, may be included in such compositions. These pharmaceutical or veterinary compositions may be injected intramuscularly, intraperitoneally, or intravenously.

The pharmaceutical composition may be formulated for topical administration. Formulations include creams, lotions, gels, powders, solutions and patches wherein the compound is dispersed or dissolved in suitable excipients.

The pharmaceutical compositions may be in any form, including, among others, tablets, pellets, capsules, aqueous or oily solutions, suspensions, emulsions, or dry powdered forms suitable for reconstitution with water or other suitable liquid medium before use, for immediate or retarded release.

The appropriate excipients and/or carriers, and their amounts, can readily be determined by those skilled in the art according to the type of formulation being prepared.

As mentioned above, the compounds of the invention having the 2,4,6,7-tetrasubstituted quinoline core and being substituted as previously defined, are inhibitors of DNMTs. For the purposes of the invention, this means that the compounds as defined above are capable of inhibiting one or more DNMTs selected from the group consisting of DNMT1, DNMT3A and DNMT3B, particularly DNMT1, with an IC₅₀ value≤10 μM, preferably ≤1 μM, more preferably ≤500 nM, when the inhibition of DNMTs is measured in enzymatic assays as the ones described in the present invention.

In one embodiment, optionally in combination with one or more features of the various embodiments described above or below, the invention relates to a compound of formula (I) which is additionally inhibitor of G9a. For the purposes of the invention, this means that the compound as defined above is capable of inhibiting G9a with an IC₅₀ value≤10 μM, preferably ≤1 μM, more preferably ≤500 nM, when the inhibition of G9a is measured in enzymatic assays as the ones described in the present invention, and also capable of inhibiting one or more DNMTs as mentioned above.

Thus, the invention relates to a compound of formula (I) or a pharmaceutical composition comprising the compound of formula (I) as defined above, for use as a medicament.

Moreover, the invention relates to a compound of formula (I) or a pharmaceutical composition comprising the compound of formula (I) as defined above, for use in the treatment of cancer, fibrosis and/or immunomodulation; in particular cancer, fibrosis and/or immunomodulation mediated by the inhibition of one or more DNMTs selected from the group consisting of DNMT1, DNMT3A and DNMT3B, particularly DNMT1.

Thus, this aspect of the invention relates to the use of a compound of formula (I) or a pharmaceutical composition comprising the compound of formula (I) as defined above, for the manufacture of a medicament for the treatment and/or prevention of cancer, fibrosis and/or immunomodulation; in particular cancer, fibrosis and/or immunomodulation mediated by the inhibition of one or more DNMTs selected from the group consisting of DNMT1, DNMT3A and DNMT3B, particularly DNMT1.

It may also be formulated as a method for the treatment and/or prevention of cancer, fibrosis and/or immunomodulation; in particular cancer, fibrosis and/or immunomodulation mediated by the inhibition of one or more DNMTs selected from the group consisting of DNMT1, DNMT3A and DNMT3B, particularly DNMT1, comprising administering an effective amount of the previously defined compound of formula (I) or a pharmaceutical composition comprising the compound of formula (I) as defined above, and one or more pharmaceutically or veterinary acceptable excipients or carriers, in a subject in need thereof, including a human.

In one embodiment, optionally in combination with one or more features of the various embodiments described above or below, cancer, fibrosis and/or immunomodulation are mediated by the dual inhibition of histone methyltransferase G9a and of one or more DNMTs selected from the group consisting of DNMT1, DNMT3A and DNMT3B, particularly DNMT1.

For the purposes of the invention, the term “treatment” of the disease refers to stopping or delaying of the disease progress, when the drug is used in the subject exhibiting symptoms of disease onset. The term “prevention” refers to stopping or delaying of symptoms of disease onset, when the drug is used in the subject exhibiting no symptoms of disease onset but having high risk of disease onset.

In one embodiment, optionally in combination with one or more features of the various embodiments described above or below, the cancer is selected from the group consisting of a hematogical cancer and a solid tumor. More particularly, the hematogical cancer is selected from the group consisting of leukemia including Acute Lymphocytic Leukemia (ALL) and acute myeloid leukemia, lymphoma including Diffuse Large B-cell lymphoma (DLBCL) and mantle cell lymphomam and multiple myeloma; and the solid tumor is selected from the group consisting of bladder cancer, breast cancer, cervical cancer, colorectal cancer, glioblastoma, hepatocarcinoma, lung cancer including small-cell lung cancer, non small-cell lung cancer, melanoma, pancreatic cancer, prostate cancer and renal cancer.

In another embodiment, optionally in combination with one or more features of the various embodiments described above or below, the cancer is selected from the group consisting of Acute Lymphocytic Leukemia (ALL), Diffuse Large B-cell lymphoma (DLBCL), bladder cancer, breast cancer, cervical cancer, colorectal cancer, glioblastoma, hepatocarcinoma, melanoma, pancreatic cancer, prostate cancer, renal cancer, small-cell lung cancer, non small-cell lung cancer, acute myeloid leukemia, mantle cell lymphoma and multiple myeloma.

Throughout the description and claims the word “comprise” and variations of thereof, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. The following examples are provided by way of illustration, and they are not intended to be limiting of the present invention. Furthermore, the present invention covers all possible combinations of particular and preferred embodiments described herein.

### Examples

General Procedure for Preparative HPLC Purification Method:

The HPLC measurement was performed using Gilson 281 from 233 pump (binary), an autosampler, and a UV detector. The fractions was detected by LC-MS. The MS detector was configured with an electrospray ionization source. The source temperature was maintained at 300-350° C.

HPLC Methods (Purification Methods):

General Conditions for Methods 1-2, 5-14, 17-42:

Reverse phase HPLC was carried out on Luna C18 (100×30 mm; 5 μm). Solvent A: water with 0.1% trifluoroacetic acid; Solvent B: acetonitrile. UV detector.

General Conditions for Methods 3, 15:

Reverse phase HPLC was carried out on Luna C18 (100×30 mm; 4 μm). Solvent A: water with 0.075% trifluoroacetic acid; Solvent B: acetonitrile with 0.075% trifluoroacetic acid. UV detector.

General Conditions for Method 4:

Reverse phase HPLC was carried out on Waters Xbridge (150×25 mm; 5 μm). Solvent A: water; Solvent B: acetonitrile. UV detector.

General Conditions for Method 16:

Reverse phase HPLC was carried out on Waters Xbridge Prep OBD (150×30 mm; 5 μm). Solvent A: aqueous NH₄HCO₃ 10 mM; Solvent B: acetonitrile. UV detector.

| Method | Gradient (at room temperature) |
|---|---|
| 1 | 15% of B to 45% of B within 12 min at 25 mL/min; then 100% B at 25 mL/min over 4 min. |
| 2 | 1% of B to 25% of B within 12 min at 20 mL/min; then 100% B at 20 mL/min over 2 min. |
| 3 | 20% of B to 40% of B within 6 min at 25 mL/min; then 40% B at 25 mL/min over 2 min. |
| 4 | 45% of B to 60% of B within 12 min at 20 mL/min; then 100% B at 20 mL/min over 5 min. |
| 5 | 3% of B to 43% of B within 10 min at 25 mL/min; then 100% B at 25 mL/min over 3 min. |
| 6 | 10% of B to 35% of B within 12 min at 20 mL/min; then 100% B at 20 mL/min over 2 min. |
| 7 | 10% of B to 40% of B within 12 min at 20 mL/min; then 100% B at 20 mL/min over 2 min. |
| 8 | 1% of B to 55% of B within 8 min at 20 mL/min; then 100% B at 20 mL/min over 2 min. |
| 9 | 2% of B to 32% of B within 12 min at 20 mL/min; then 100% B at 20 mL/min over 2 min. |
| 10 | 10% of B to 30% of B within 12 min at 20 mL/min; then 100% B at 20 mL/min over 2 min. |
| 11 | 3% of B to 33% of B within 12 min at 25 mL/min; then 100% B at 25 mL/min over 3 min. |
| 12 | 5% of B to 35% of B within 15 min at 11 mL/min; then 100% B at 11 mL/min over 10 min. |
| 13 | 1% of B to 50% of B within 10 min at 20 mL/min; then 100% B at 20 mL/min over 2 min. |
| 14 | 8% of B to 35% of B within 10 min at 80 mL/min; then 100% B at 80 mL/min over 20 min. |
| 15 | 25% of B to 45% of B within 6 min at 20 mL/min; then 40% B at 25 mL/min over 3 min. |
| 16 | 5% of B to 35% of B within 12 min at 20 mL/min; then 100% B at 20 mL/min over 2 min. |
| 17 | 8% of B to 35% of B within 10 min at 80 mL/min; then 100% B at 80 mL/min over 20 min. |
| 18 | 15% of B to 45% of B within 12 min at 20 mL/min; then 100% B at 20 mL/min over 2 min. |
| 19 | 15% of B to 45% of B within 2 min at 20 mL/min; then 100% B at 20 mL/min over 12 min. |
| 20 | 1% of B to 50% of B within 10 min at 20 mL/min; then 100% B at 20 mL/min over 2 min. |
| 21 | 3% of B to 40% of B within 12 min at 20 mL/min; then 100% B at 20 mL/min over 2 min. |
| 22 | 1% of B to 20% of B within 2 min at 20 mL/min; then 100% B at 20 mL/min over 2 min. |
| 23 | 5% of B to 30% of B within 12 min at 80 mL/min; then 100% B at 80 mL/min over 2 min. |
| 24 | 25% of B to 50% of B within 12 min at 20 mL/min; then 100% B at 20 mL/min over 2 min. |
| 25 | 1% of B to 50% of B within 12 min at 20 mL/min; then 100% B at 20 mL/min over 2 min. |
| 26 | 1% of B to 26% of B within 12 min at 20 mL/min; then 100% B at 20 mL/min over 2 min. |
| 27 | 15% of B to 25% of B within 12 min at 20 mL/min; then 100% B at 20 mL/min over 2 min. |
| 28 | 20% of B to 40% of B within 12 min at 20 mL/min; then 100% B at 20 mL/min over 2 min. |
| 29 | 25% of B to 55% of B within 12 min at 20 mL/min; then 100% B at 20 mL/min over 3 min. |
| 30 | 10% of B to 25% of B within 12 min at 20 mL/min; then 100% B at 20 mL/min over 2 min. |
| 31 | 1% of B to 21% of B within 12 min at 20 mL/min; then 100% B at 20 mL/min over 2 min. |
| 32 | 13% of B to 23% of B within 12 min at 20 mL/min; then 100% B at 20 mL/min over 2 min. |
| 33 | 1% of B to 35% of B within 12 min at 20 mL/min; then 100% B at 20 mL/min over 2 min. |
| 34 | 5% of B to 40% of B within 12 min at 20 mL/min; then 100% B at 20 mL/min over 2 min. |
| 35 | 10% of B to 40% of B within 12 min at 20 mL/min; then 100% B at 20 mL/min over 5 min. |
| 36 | 20% of B to 60% of B within 12 min at 20 mL/min; then 100% B at 20 mL/min over 2 min. |
| 37 | 10% of B to 45% of B within 12 min at 20 mL/min; then 100% B at 20 mL/min over 5 min. |
| 38 | 5% of B to 55% of B within 12 min at 20 mL/min; then 100% B at 20 mL/min over 2 min. |
| 39 | 18% of B to 38% of B within 12 min at 20 mL/min; then 100% B at 20 mL/min over 2 min. |
| 40 | 10% of B to 45% of B within 12 min at 20 mL/min; then 100% B at 20 mL/min over 2 min. |
| 41 | 15% of B to 30% of B within 12 min at 20 mL/min; then 100% B at 20 mL/min over 2 min. |
| 42 | 16% of B to 46% of B within 12 min at 20 mL/min; then 100% B at 20 mL/min over 4 min. |

General Procedure for HPLC Analysis

HPLC-analysis was performed using a Shimadzu LC-20AB or LC-20AD with a Luna-C18(2) column (2.0×50 mm, 5 μm) at 40° C. and UV detection.

Method 1:

Solvent A: water with 0.056% TFA; Solvent B: acetonitrile with 0.056% TFA. Gradient: After 0.01 minutes at the initial condition of 100% A, solvent B was increased to 60% over 4 minutes, maintained at 60% for 0.8 minutes, then a linear gradient to initial conditions was applied for 0.02 minutes and maintained for 0.68 minutes to re-equilibrate the column, giving a cycle time of 5.90 minutes. Flow rate was 0.8 mL/min from 0.01 to 5.21 minutes, increased to 1.2 mL/min in 0.02 minutes and maintained until the end of the run.

Method 2:

Solvent A: water with 0.056% TFA; Solvent B: acetonitrile with 0.056% TFA. Gradient: After 0.1 minutes at the initial condition of 90% A and 10% B, solvent B was increased to 80% over 4 minutes, maintained at 80% for 0.9 minutes, then a linear gradient to initial conditions was applied for 0.02 minutes and maintained for 0.58 minutes to re-equilibrate the column, giving a cycle time of 5.50 minutes. Flow rate was 0.8 mL/min from 0.01 to 4.90 minutes, increased to 1.2 mL/min in 0.03 minutes and maintained until the end of the run.

Method 3:

Solvent A: water with 0.037% TFA; Solvent B: acetonitrile with 0.018% TFA. Gradient: After 0.01 minutes at the initial condition of 90% A and 10% B, solvent B was increased to 80% over 4 minutes, maintained at 80% for 0.9 minutes, then a linear gradient to initial conditions was applied for 0.02 minutes and maintained for 0.58 minutes to re-equilibrate the column, giving a cycle time of 5.50 minutes. Flow rate was 0.8 mL/min from 0.01 to 4.90 minutes, increased to 1.2 mL/min in 0.03 minutes and maintained until the end of the run.

The following abbreviations have been used in the examples: HPLC: High-performance liquid chromatography; TLC: thin layer chromatography; MW: microwaves; calc.: calculated; rt: room temperature; Rt: Retention time; min: minute; Boc: tert-butoxycarbonyl; DMAP: 4-Dimethylaminopyridine; DCM: dichloromethane; DMF: dimethylformamide; DMSO: dimethylsulfoxide; eq: equivalent; ESI-MS: electrospray ionization mass spectrometry; Et₃N: triethylamine; TFA: trifluoroacetic acid; THF: tetrahydrofuran; DEAD: diethylazodicarboxylate; BINAP: 2,2′bis(diphenylphospinio)-1,1′-binaphthyl; EtOAc: ethyl acetate; EtOH: ethanol; MeOH: methanol; t-BuOH: tert-butanol; n-BuLi: n-Butyllithium; Ph: phenyl; PTSA: p-Toluenesulfonic acid; UV: ultraviolet; MsCl: methanesulphonyl chloride; PG: protective group; Bn: Benzyl; Prep: Preparative; i-PROH: isopropyl alcohol.

### Preparation of Reagent R-02b: 2-(5-ethyl-2-furyl)-4,4,5,5-tetramethyl-1,3,2-dioxaborolane

To a solution of 2-ethylfuran (1.92 g, 20 mmol) in THF (100 mL) was added n-BuLi (8.8 mL, 22 mmol) at −78° C. slowly and the mixture was stirred at −25° C. for 2 hours. Then 2-isopropoxy-4,4,5,5-tetramethyl-1,3,2-dioxaborolane (3.72 g, 20 mol) was added into the reaction mixture at −78° C. and the solution was stirred at room temperature overnight. The reaction mixture was quenched with water and extracted with EtOAC. The organic layer was concentrated under vacuum to give the desired reagent R-02b (1 g, 22%). ESI-MS (M+1): 223 calc. for C₁₂H₁₉BO₃: 222.1.

### Preparation of Reagent R-02d: 2-methyl-5-(4,4,5,5-tetramethyl-1,3,2-dioxaborolan-2-yl)-1H-pyrrole

A 40-mL flask assembled a magnetic stirring bar, a septum inlet, and a condenser was charged with (1Z,5Z)-cycloocta-1,5-diene; 2,4-dimethyl-BLAHbicyclo[1.1.0]butane (160 mg, 0.241 mmol), 4-tert-butyl-2-(4-tert-butyl-2-pyridyl)pyridine (130 mg, 0.483 mmol) and 4,4,5,5-tetramethyl-2-(4,4,5,5-tetramethyl-1,3,2-dioxaborolan-2-yl)-1,3,2-dioxaborolane (1.53 g, 6.04 mmol) and then flushed with nitrogen. Dry hexane (60 mL) and 2-methyl-1H-pyrrole (980 mg, 12.08 mmol) were added, and the mixture was stirred at 30° C. for 2 hours. Then the reaction mixture was concentrated under vacuum and purified by column chromatography to give reagent R-02d (1.1 g, 44%) as yellow solid. ESI-MS (M+1): 208.1 calc. for C₁₁H₁₈BNO₂: 207.1.

### Preparation of Reagent R-03d: N,7-dimethyl-7-azaspiro[3.5]nonan-2-amine

To a solution of commercially available tert-butyl 2-oxo-7-azaspiro[3.5]-nonane-7-carboxylate (1.90 g, 7.94 mmol) in 1,2-dichloromethane (20 mL) were added NaBH₃CN (1.50 g, 23.82 mmol), N-methyl-1-phenyl-methanamine (1.15 g, 9.53 mmol, 1.23 mL) and CH₃COOH (477 mg, 7.94 mmol, 0.454 mL) at 20° C. under N₂ and the reaction mixture was stirred at 25° C. for 16 hours. Then, the reaction mixture was concentrated under reduced pressure at 40° C. The residue was diluted with water and extracted with ethyl acetate. The combined organic phase was washed with brine, dried with anhydrous Na₂SO₄, filtered and concentrated under vacuum to give a crude that was purified by prep-HPLC (General Procedure, Method 1) to give intermediate tert-butyl 2-[benzyl(methyl)amino]-7-azaspiro[3.5]nonane-7-carboxylate (1.00 g, 36%) as a yellow solid. Then a mixture of this intermediate (1.00 g, 2.90 mmol) in HCl/EtOAc (5 mL, 2 N) was stirred at 20° C. for 2 hours. The mixture was concentrated under reduced pressure at 40° C. to give crude N-benzyl-N-methyl-7-azaspiro[3.5]nonan-2-amine (700 mg, 85%) as colorless oil which was used in the next step without further purification. To a solution of this intermediate (300 mg, 1.23 mmol) in MeOH (2 mL) were added NaBH₃CN (231 mg, 3.68 mmol), (HCHO)ₙ (332 mg, 3.68 mmol) and HCOOH (177 mg, 3.68 mmol) at 20° C. under N₂ and the reaction mixture was stirred at 60° C. for 16 hours. Then, the reaction mixture was filtered and the filtrate was concentrated under vacuum to give the crude product which was further purified by prep-HPLC (General Procedure, Method 2) to afford intermediate N-benzyl-N,7-dimethyl-7-azaspiro[3.5]nonan-2-amine (150 mg, 47%) as colorless oil. Finally, to a solution of this intermediate (190 mg, 0.735 mmol) in MeOH (20 mL) was added Pd/C (10%, 50 mg) under N₂ atmosphere. The suspension was degassed and purged with H₂ for 3 times. The reaction mixture was stirred under H₂ (50 Psi) at 30° C. for 2 hours. Then, the reaction mixture was filtered and the filtrate was concentrated to dryness to give reagent R-03d (100 mg, 81%) as colorless oil. ESI-MS (M+1): 169.2 calc. for C₁₀H₂₀N₂: 168.2.

### Preparation of Reagent R-03e: 7-isopropyl-N-methyl-7-azaspiro[3.5]nonan-2-amine

A solution of N-benzyl-N-methyl-7-azaspiro[3.5]nonan-2-amine (300 mg, 1.23 mmol) in i-PROH (10 mL), prepared as described above for reagent R-03d, were added acetone (429 mg, 7.38 mmol, 0.542 mL), NaBH₃CN (232 mg, 3.69 mmol) and CH₃COOH (222 mg, 3.69 mmol, 0.211 mL) at 20° C. under N₂. The mixture was stirred at 60° C. for 16 hours. The mixture was filtered and the filtrate was concentrated under vacuum. The crude product was purified by prep-HPLC (General Procedure, Method 2) to afford intermediate N-benzyl-7-isopropyl-N-methyl-7-azaspiro[3.5]nonan-2-amine (200 mg, 698.20 μmol, 56.76% yield) as a yellow oil. Finally, to a solution of this intermediate (120 mg, 418.92 μmol) in MeOH (15 mL) was added Pd/C (30 mg, 10%) under N₂ atmosphere. The suspension was degassed and purged with H₂ for 3 times. Then the mixture was stirred under H₂ (50 Psi) at 30° C. for 2 hours. The reaction mixture was filtered and the filtrate was concentrated to dryness to give reagent R-03e (50 mg, 254.67 μmol, 60.79% yield) as a colorless oil. ESI-MS (M+1): 196.2 calc. for C₁₂H₂₄N₂: 197.3.

### Preparation of Reagent R-03g: N,2-dimethyl-2-azaspiro[3.5]nonan-7-amine

To a solution of commercially available tert-butyl 2-oxo-7-azaspiro[3.5]nonane-7-carboxylate (4 g, 16.71 mmol) in MeOH (100 mL) were added NaBH₃CN (1.58 g, 25.07 mmol), 1-(2,4-dimethoxyphenyl)-N-methyl-methanamine (3.03 g, 16.71 mmol) and CH₃COOH (100 mg, 1.67 mmol, 95.59 μL) at 20° C. under N₂. The mixture was stirred at 50° C. for 16 hours and then it was cooled to 20° C. and concentrated in reduced pressure at 40° C. The residue was poured into water (100 mL) and extracted with ethyl acetate (100 mL×3). The combined organic phase was washed with brine (100 mL×2), dried with anhydrous Na₂SO₄, filtered and concentrated in vacuum to afford crude intermediate tert-butyl 2-[(2,4-dimethoxyphenyl)methyl-methyl-amino]-7-azaspiro[3.5]nonane-7-carboxylate (7 g) as yellow oil. A mixture of this intermediate (7 g, 17.30 mmol) in HCl/MeOH (100 mL, 2M) was stirred at 18° C. for 3 hours. The mixture was concentrated in reduced pressure at 40° C. to afford crude intermediate N-[(2,4-dimethoxyphenyl)methyl]-N-methyl-7-azaspiro[3.5]nonan-2-amine (6 g) as yellow oil. Then, a mixture of this intermediate (1.00 g, 3.28 mmol), HCOOH (158 mg, 3.43 mmol), (HCHO)ₙ (888 mg, 9.85 mmol) and NaBH₃CN (679 mg, 10.81 mmol) in MeOH (20 mL) was degassed and purged with N₂ for 3 times, and then the mixture was stirred at 60° C. for 16 hours under N₂ atmosphere. The mixture was concentrated in reduced pressure at 40° C. and the residue was poured into water (50 mL) and extracted with ethyl acetate (50 mL×3). The combined organic phase was washed with brine (50 mL×2), dried with anhydrous Na₂SO₄, filtered and concentrated in vacuum to give the crude product, which was purified by prep-HPLC (General Procedure, Method 31) to afford N-[(2,4-dimethoxyphenyl)methyl]-N,7-dimethyl-7-azaspiro[3.5]nonan-2-amine (400 mg, 1.26 mmol, 38.30% yield) as colorless oil. Finally, to a solution of this intermediate (400 mg, 1.26 mmol) in MeOH (50 mL) was added Pd/C (10%, 50 mg) under H₂ atmosphere. The suspension was degassed and purged with H₂ for 3 times. The mixture was stirred under H₂ (50 Psi) at 30° C. for 16 hours. The reaction mixture was filtered and the filtrate was concentrated to give reagent R-03g (200 mg, 1.19 mmol, 94.33% yield) as colorless oil. ESI-MS (M+1): 169.3 calc. for C₁₀H₂₀N₂: 168.2.

### Preparation of Reagent R-04a: tert-butyl 4-(methylsulfonyloxymethyl)-piperidine-1-carboxylate

To a solution of commercially available tert-butyl 4-(hydroxymethyl)piperidine-1-carboxylate (3.00 g, 13.93 mmol) in DCM (100 mL) was added Et₃N (4.23 g, 41.79 mmol, 5.79 mL). Then MsCl (2.39 g, 20.90 mmol, 1.62 mL) was added to the above solution at 0° C. under N₂. The mixture was stirred at 20° C. for 16 hours. Then, the reaction mixture was poured into water and extracted with DCM. The combined organic phase was washed with brine, dried with anhydrous Na₂SO₄, filtered and concentrated under vacuum to give reagent R-04a (3.00 g, 73%) as a white solid. ESI-MS (M+1-56): 238.1 calc. for C₁₂H₂₃NO₅: 293.1.

### Preparation of Reagent R-06c: tert-butyl 3-(methylamino)-7-azaspiro[3.5]nonane-7-carboxylate

A mixture of commercially available tert-butyl 3-oxo-7-azaspiro[3.5]nonane-7-carboxylate (500 mg, 2.09 mmol), CH₃NH₂ in MeOH (1.15 g, 10.45 mmol, 30% purity), NaBH₃CN (197 mg, 3.14 mmol) and CH₃COOH (188.26 mg, 3.14 mmol, 179.30 μL) in MeOH (20 mL) was degassed and purged with N₂ for 3 times, and then the mixture was stirred at 40° C. for 12 hours under N₂ atmosphere. The reaction mixture was filtrated and the filtrate was concentrated under vacuum to give a residue, which was purified by column chromatography (SiO₂, Petroleum ether/Ethyl acetate=100:1 to 5:1; retention factor of 0.19) to afford reagent R-06c (280.00 mg, 1.10 mmol, 52.67% yield) as a white solid. ESI-MS (M+1): 255.3 calc. for C₁₄H₂₆N₂O₂: 254.2.

### Preparation of Reagent R-06d: tert-butyl 4-(1-amino-1-methyl-ethyl)piperidine-1-carboxylate

A suspension of anhydrous CeCl₃ (27.08 g, 109.86 mmol) in anhydrous THF (150 mL) was heated to 45° C. for 3 hours. The slurry was cooled to rt and treated with commercially available tert-butyl 4-cyanopiperidine-1-carboxylate (7.0 g, 33.29 mmol). After further cooling to −78° C., a 1.0 M solution of MeLi in THF (99.87 mL, 99.87 mmol) was added drop-wise over 30 min to give a brown slurry, which was aged for additional 30 min. Then the reaction mixture was stirred at 25° C. for 12 hours. The reaction mixture was quenched by addition of NH₃.H₂O 70 mL at −78° C., and then warmed to 25° C. for 1 hour. The reaction mixture was filtrated and the filtrate was concentrated under vacuum to give a residue, the residue was extracted with EtOAc (100 mL×3).

The combined organic layers were washed with water (50 mL×1), dried over Na₂SO₄, filtered and concentrated under reduced pressure to give a residue, which was purified by prep-HPLC (General Procedure, Method 34) to afford reagent R-06d (1.4 g, 5.78 mmol, 17.3% yield) as a pink solid. ESI-MS (M+H-56): 187.2.3 calc. for C₁₃H₂₆N₂O₂: 242.2.

### Preparation of Reagent R-08a (also referred herein to as R-04b): tert-butyl 6-methylsulfonyloxy-2-azaspiro[3.3]heptane-2-carboxylate

To a solution of commercially available tert-butyl 6-hydroxy-2-azaspiro[3.3]heptane-2-carboxylate (2.4 g, 11.25 mmol) in DCM (30 mL) was added Et₃N (1.71 g, 16.88 mmol) at 18° C. under N₂ and MsCl (1.29 g, 11.25 mmol) at 0° C. The mixture was stirred at 0° C. for 1 hour under N₂. Then, the reaction mixture was poured into water and the organic phase was separated. The aqueous phase was extracted with DCM and the combined organic phase was washed with brine, dried with anhydrous Na₂SO₄, filtered and concentrated in vacuum to give reagent R-08a (3.4 g, 100% crude) as yellow solid. ESI-MS (M+1): 292.2 calc. for C₁₂H₂₁NO₅S: 291.1.

### Preparation of Reagent R-10a: tert-butyl 4-[(4,4,5,5-tetramethyl-1,3,2-dioxaborolan-2-yl)methylene]piperidine-1-carboxylate

To a solution of tert-butyl 4-methylenepiperidine-1-carboxylate (500 mg, 2.53 mmol) in DCM (20 mL) was added [1,3-bis(2,4,6-trimethylphenyl)imidazolidin-2-ylidene]-dichloro-[(2-isopropoxyphenyl)methylene]ruthenium (159 mg, 0.25 mmol) and 4,4,5,5-tetramethyl-2-vinyl-1,3,2-dioxaborolane (390 mg, 2.53 mmol) at 20° C. under N₂. The mixture was stirred at 65° C. for 16 hours and then, the reaction mixture was poured into water and extracted with DCM. The combined organic phase was washed with brine, dried with anhydrous Na₂SO₄, filtered and concentrated under vacuum to give a residue which was purified by silica gel column chromatography to give reagent R-10a (140 mg, 17%) as colorless oil.

Synthetic Route 1

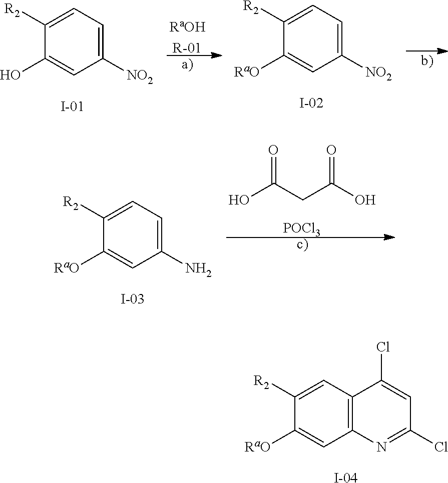

Conditions: a) PPh₃ (2.0 eq), DEAD (2.0 eq), R-01 (1.0 eq), 0° C., then rt, 5 h; b) Pd/C, MeOH, H₂ (1 atm), rt, 3 h; c) POCl₃, malonic acid (1.1 eq), rt, 4 h, then 90° C., overnight.

In the scheme above R₂ is O(C₁-C₆)alkyl and R^{a} is a hydrocarbon chain which contains nitrogen and/or oxygen atoms.

### Preparation of Intermediate I-02a: 1-[3-(2-methoxy-5-nitro-phenoxy)propyl]-pyrrolidine

To a solution of commercially available 2-methoxy-5-nitro-phenol (I-01a, 19.6 g, 0.12 mol) in THF (200 mL) was added PPh₃ (61 g, 0.23 mol), commercially available 3-pyrrolidin-1-yl-propan-1-ol (R-01a, 15 g, 0.12 mol) and DEAD (40 g, 0.23 mol) at 0° C. and the solution was stirred at room temperature for 5 hours. The reaction mixture was concentrated and extracted with EtOAc. The combined organic layers were washed with brine, dried over anhydrous Na₂SO₄, filtered and concentrated to give the crude product which was purified by column chromatography to give intermediate I-02a (14 g, 42%) as a yellow solid. ESI-MS (M+1): 281 calc. for C₁₄H₂₀N₂O₄: 280.1.

### Preparation of Intermediate I-03a: 4-methoxy-3-(3-pyrrolidin-1-ylpropoxy)aniline

To a solution of intermediate I-02a (14 g, 0.05 mol) in MeOH (200 mL) was added Pd/C (3 g). The solution was stirred at room temperature for 3 hours in H₂ atmosphere (1 atm). Then, the solution was filtrated and concentrated to give intermediate I-03a (12 g, 96%) as a yellow oil. ESI-MS (M+1): 251 calc. for C₁₄H₂₂N₂O₂: 250.1.

### Preparation of Intermediate I-04a: 2,4-dichloro-6-methoxy-7-(3-pyrrolidin-1-ylpropoxy)quinoline

To a solution of intermediate I-03a (12.4 g, 0.049 mol) in POCl₃ (200 mL) was added malonic acid (5.67, 0.055 mol) at room temperature. After stirring at room temperature for 4 hours, the solution was heated at 90° C. overnight. Then, the solution was concentrated and poured into ice-water and extracted with EtOAc. The combined organic layers were washed with brine, dried over anhydrous Na₂SO₄, filtered and concentrated to give intermediate I-04a (10 g, 58%) as a pale yellow solid. ESI-MS (M+1): 355 calc. for C₁₇H₂₀Cl₂N₂O₂: 354.1.

Synthetic Route 2

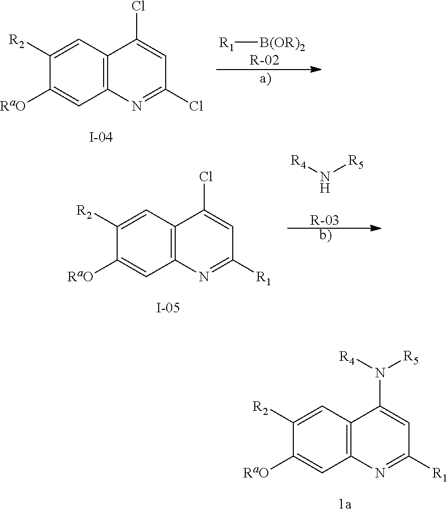

Conditions: a) R-02 (1.0-1.2 eq), Pd(PPh₃)₄(0.1-0.2 eq), Na₂CO₃ or K₂CO₃ (2.0-3.0 eq), 1,4-dioxane/H₂O (1:1, 3:1, 10:1 or 15:1), 90-120° C., MW, 2-4 h or conventional heating 16 h; b) R-03 (1.0-10 eq), PTSA (1.1-1.5 eq), t-BuOH, 120-130° C., MW or conventional heating, 16-72 h.

In the scheme above R₂ is O(C₁-C₆)alkyl, R^{a} is a hydrocarbon chain which contains nitrogen and/or oxygen atoms, R₁ is aryl or heteroaryl, R is H, (C₁-C₆)alkyl or, alternatively, two R groups together with the B atom to which they are attached may form a cycle, and R₄ and R₅ are H, a cycle (Cy) or a hydrocarbon chain which optionally contains nitrogen oxygen and/or fluor atoms.

### Preparation of Intermediate I-05a: 4-chloro-6-methoxy-2-(5-methyl-2-furyl)-7-(3-pyrrolidin-1-ylpropoxy)quinoline

To a solution of intermediate I-04a (600 mg, 1.7 mmol) in 1,4-dioxane/H₂O (15:1, 16 mL) were added Na₂CO₃ (0.54 g, 5.1 mmol), Pd(PPh₃)₄(0.22 g, 0.17 mmol) and commercially available 4,4,5,5-tetramethyl-2-(5-methyl-2-furyl)-1,3,2-dioxaborolane (R-02a, 0.39 g, 1.87 mmol). The solution was stirred at 110° C. for 4 hours under Microwave. Then, the mixture was quenched with water and extracted with EtOAc. The combined organic phase was washed with brine, dried over anhydrous Na₂SO₄ and concentrated to give the crude product which was purified by prep-HPLC (General procedure, Method 3) to give intermediate I-05a (400 mg, 59%) as a yellow solid. ESI-MS (M+1): 401.2 calc. for C₂₂H₂₅ClN₂O₃: 400.1.

Following the same synthetic route for intermediate I-05a and using the same reagents and intermediates unless otherwise indicated in the table below, the following intermediates were obtained:

| Intermediate | | | |
|---|---|---|---|
| I-05 | Yield | [M + 1]⁺ | Intermediate/reagent |
| I-05b | 85% | 415.3 | I-04a/2-(5-ethyl-2-furyl)-4,4,5,5-tetramethyl-1,3,2- |
| | | | dioxaborolane (R-02b) |
| I-05c | 78% | 415.3 | I-04a/2-(2,5-dimethyl-3-furyl)-4,4,5,5-tetramethyl- |
| | | | 1,3,2-dioxaborolane (R-02c) |
| I-05d | 38% | 400.2 | I-04a/2-methyl-5-(4,4,5,5-tetramethyl-1,3,2- |
| | | | dioxaborolan-2-yl)-1H-pyrrole (R-02d) |
| I-05e | 53% | 437.2 | I-04a/benzofuran-5-ylboronic acid (R-02e) |
| I-05f | 89% | 401.1 | I-04a/2-(cyclohexen-1-yl)-4,4,5,5-tetramethyl-1,3,2- |
| | | | dioxaborolane (R-02f) |
| I-05g | 54% | 397.1 | I-04a/4,4,5,5-tetramethyl-2-phenyl-1,3,2- |
| | | | dioxaborolane (R-02g) |

### Preparation of Compound I-01: N-benzyl-6-methoxy-N-methyl-2-(5-methyl-2-furyl)-7-(3-pyrrolidin-1-ylpropoxy)quinolin-4-amine

To a solution of intermediate I-05a (100 mg, 249 μmol) and commercially available N-methyl-1-phenyl-methanamine (R-03a, 33 mg, 274 μmol) in t-BuOH (5 mL) was added p-toluenesulfonic acid (47 mg, 274 μmol) and the mixture was stirred at 120° C. for 72 hours in microwave reactor. Then, the reaction was concentrated in vacuum to give a residue which was purified by prep-HPLC (General procedure, Method 4) to give compound I-01 (11 mg, 9%) as a yellow solid. ESI-MS (M+1): 486.3 calc. for C₃₀H₃₅N₃O₃: 485.3. HPLC analytical method 1, Rt=3.34 min.

Following the same synthetic route for compound I-01 and using the same reagents and intermediates unless otherwise indicated in the table below, the following compounds were obtained:

| | Method | | Method | | |
|---|---|---|---|---|---|
| | Prep- | | Analytical | | |
| Example | HPLC | [M + 1]⁺ | HPLC | Rt | Intermediate/Reagent |
| 1-02 | 5 | 493.3 | 2 | 1.40 | I-05a/N,1-dimethylpiperidin-4- |
| | | | | | amine (R-03b) |
| 1-03 | 5 | 507.3 | 2 | 1.39 | I-05a/N-methyl-1-(1-methyl-4- |
| | | | | | piperidyl)methanamine (R-03c) |
| 1-04 | 6 | 521.4 | 2 | 1.44 | I-05b/N-methyl-1-(1-methyl-4- |
| | | | | | piperidyl)methanamine (R-03c) |
| 1-05 | 7 | 507.4 | 2 | 1.37 | I-05c/N,1-dimethylpiperidin-4- |
| | | | | | amine (R-03b) |
| 1-06 | 8 | 506.4 | 2 | 1.58 | I-05d/N-methyl-1-(1-methyl-4- |
| | | | | | piperidyl)methanamine (R-03c) |
| 1-07 | 9 | 492.3 | 2 | 1.50 | I-05d/N,1-dimethylpiperidin-4- |
| | | | | | amine (R-03b) |
| 1-09 | 27 | 561.4 | 3 | 1.52 | I-05a/7-isopropyl-N-methyl-7- |
| | | | | | azaspiro[3.5]nonan-2-amine (R- |
| | | | | | 03e) |
| 1-13 | 28 | 463.3 | 3 | 2.27 | I-05d/N- |
| | | | | | methylcyclopentanamine (R-03f) |
| 1-17 | 29 | 464.2 | 3 | 2.41 | I-05a/N- |
| | | | | | methylcyclopentanamine (R-03f) |
| 1-18 | 30 | 533.4 | 3 | 1.56 | I-05a/N,2-dimethyl-2- |
| | | | | | azaspiro[3.5]nonan-7-amine (R- |
| | | | | | 03g) |
| 1-19 | 32 | 521.4 | 3 | 1.64 | I-05c/N-methyl-1-(1-methyl-4- |
| | | | | | piperidyl)methanamine (R-03c) |
| 1-22 | 38 | 503.3 | 3 | 1.38 | I-05g/N-methyl-1-(1-methyl-4- |
| | | | | | piperidyl)methanamine (R-03c) |
| 1-11 | 28 | 409.2 | 3 | 1.86 | I-05d/N-methylmethanamine |
| | | | | | (R-03h) |
| 1-12 | 39 | 410.2 | 3 | 1.69 | I-05a/N-methylmethanamine |
| | | | | | (R-03h) |

### Preparation of Compound I-21: 2-cyclohexyl-6-methoxy-N-methyl-N-[(1-methyl-4-piperidyl)methyl]-7-(3-pyrrolidin-1-ylpropoxy)quinolin-4-amine

Following the same synthetic route as for compound I-01, and starting from intermediate I-05f and reagent N-methyl-1-(1-methyl-4-piperidyl)methanamine (R-03c), intermediate compound 2-(cyclohexen-1-yl)-6-methoxy-N-methyl-N-[(1-methyl-4-piperidyl)methyl]-7-(3-pyrrolidin-1-ylpropoxy)quinolin-4-amine (60 mg, crude) was obtained as a yellow solid. ESI-MS (M+1): 507.4 calc. for C₃₁H₄₆N₄O₂: 506.4. Then, to a solution of this intermediate (60 mg, 118.41 μmol) in MeOH (10 mL) was added Pd/C (10%, 10 mg) under H₂ atmosphere. The suspension was degassed and purged with H₂ for 3 times. The mixture was stirred under H₂ (30 Psi) at 20° C. for 3 hours, filtered and concentrated in vacuum. The crude product was purified by prep-HPLC (General procedure, Method 37) to give compound I-21 (21.8 mg, 36% yield) as yellow syrup. ESI-MS (M+1): 509.5 calc. for C₃₁H₄₈N₄O₂: 508.4. HPLC analytical method 3, Rt=1.63 min.

### Preparation of Compound I-23: 6-methoxy-N-methyl-2-phenyl-N-(4-piperidylmethyl)-7-(3-pyrrolidin-1-ylpropoxy)quinolin-4-amine

Following the same synthetic route as for compound I-01, and starting from intermediate I-05g and commercially available reagent tert-butyl 4-(methylaminomethyl)piperidine-1-carboxylate (R-03g, also referred herein to as R-06f), intermediate compound tert-butyl 4-[[[6-methoxy-2-phenyl-7-(3-pyrrolidin-1-ylpropoxy)-4-quinolyl]-methyl-amino]methyl]piperidine-1-carboxylate was obtained as yellow solid. ESI-MS (M+1): 589.4 calc. for C₃₅H₄₈N₄O₄: 588.4. Then, to a solution of this intermediate (70 mg, 99.60 umol, 1.00 eq, TFA) in DCM (4 mL) was added TFA (1.08 g, 9.45 mmol, 699.98 uL) at 0° C. The mixture was stirred at 20° C. for 2 hours and concentrated in reduced pressure at 40° C. The crude product was purified by prep-HPLC (General procedure, Method 10) to give compound I-23 (19.2 mg, 39% yield) as yellow solid. ESI-MS (M+1): 489.3 calc. for C₃₀H₄₀N₄O₂: 488.3. HPLC analytical method 3, Rt=1.37 min.

Synthetic Route 3

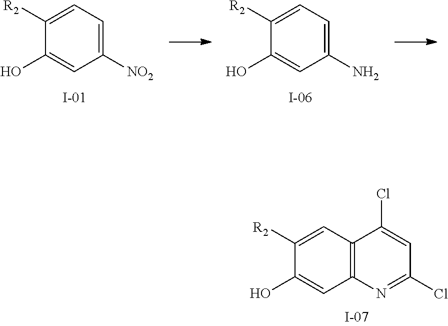

Conditions: a) Pd/C, MeOH, H₂ (40 psi), rt, 24 h; b) POCl₃, malonic acid (2.0 eq), rt, 10 minutes, then 95° C., 12 h.

In the scheme above R₂ O(C₁-C₆)alkyl.

### Preparation of Intermediate I-06a: 5-amino-2-methoxy-phenol

To a solution of commercially available 2-methoxy-5-nitro-phenol: 1-01a (40 g, 236.5 mmol) in MeOH (300 mL) was added Pd/C (3 g) under Ar. The suspension was degassed under vacuum and purged with H₂ several times. The reaction mixture was stirred under H₂ (40 psi) at room temperature for 24 hours. Then, the mixture was filtered and the filtrate was concentrated to give intermediate I-06a (25g, 76%) as a yellow solid. ESI-MS (M+1): 140.1 calc. for C₇H₉NO₂: 139.06.

### Preparation of Intermediate I-07a: 2,4-dichloro-6-methoxy-quinolin-7-ol

To a mixture of intermediate I-06a (4.91 g, 35.29 mmol) and malonic acid (7.34 g, 70.57 mmol) was added POCl₃ (70 mL) in one portion at room temperature under N₂ atmosphere. The mixture was stirred at room temperature for 10 minutes and then heated at 95° C. for 12 hours. Then, the mixture was cooled to room temperature and concentrated under reduced pressure at 60° C., to remove POCl₃. The residue was poured into water and stirred for 20 minutes. The aqueous phase was extracted with EtOAc. The organic phase was separated, washed with brine, dried with anhydrous Na₂SO₄, filtered and concentrated in vacuum. The residue was purified by silica gel chromatography to afford intermediate I-07a (2.10 g, 24% yield). ESI-MS (M+1): 244.0 calc. for C₂₃H₂₇ClN₂O₃: 242.9.

Synthetic Route 4

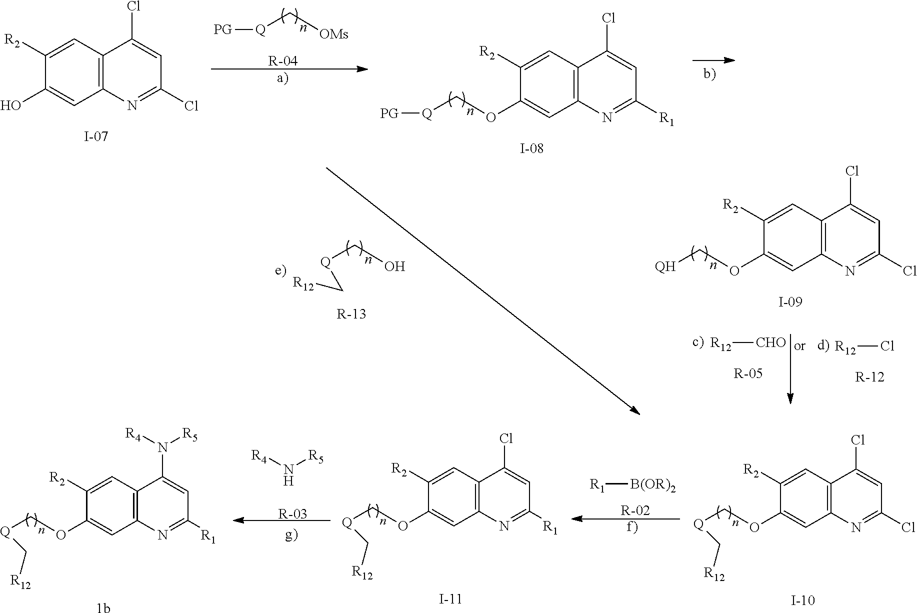

Conditions: a) Cs₂CO₃ (1.0-2.0 eq), R-04 (1.5 eq), DMF, 70-100° C., 16 h; b) HCl/EtOAc (2.0 M), 20° C., 2-4 h or TFA/DCM, 20° C., 1-2 h; c) HCOOH (1.0 eq), R-05 (3.0 eq), NaBH₃CN (3.0 eq), MeOH, 60° C., 16 h; d) KI, K₂CO₃, CH₃CN, 85° C., R-12 e) PPh₃ (1.30 eq), DEAD (1.30 eq), R-13 (1 eq), 20° C., 16 h; f) K₂CO₃ (2.0 eq), R-02 (0.6-0.7 eq), Pd(PPh₃)₄(0.1 eq), 1,4-dioxane/H₂O (10:2), 110° C., 16 h; g) PTSA (1.5 eq), R-03 (1.5 eq), t-BuOH or n-BuOH, 110-130° C., 16 h.

In the scheme above R₂ is O(C₁-C₆)alkyl, R^{a} is a hydrocarbon chain which contains nitrogen and/or oxygen atoms, R₁ is aryl or heteroaryl, R is H, (C₁-C₆)alkyl or, alternatively, two R groups together with the B atom to which they are attached may form a cycle, and R₄ and R₅ are H, a cycle (Cy) or a hydrocarbon chain which optionally contains nitrogen oxygen and/or fluor atoms, R₁₂ is H or (C₁-C₆)alkyl, PG is a protective group, ( )ₙ is a —CH2-linker repeated n times and Q is a moiety containing a nitrogen atom.

### Preparation of Intermediate I-08a: tert-butyl 4-[(2,4-dichloro-6-methoxy-7-quinolyl)oxymethyl]piperidine-1-carboxylate

To a solution of intermediate I-07a (400 mg, 1.64 mmol) in DMF (20 mL) were added Cs₂CO₃ (534 mg, 1.64 mmol) and tert-butyl 4-(methylsulfonyloxymethyl)piperidine-1-carboxylate (R-04a, 721 mg, 2.46 mmol) at 20° C. under N₂.The mixture was stirred at 70° C. for 16 hours and then, the mixture was concentrated under reduced pressure at 40° C. The resulting residue was poured into water and extracted with ethyl acetate. The combined organic phase was washed with brine, dried with anhydrous Na₂SO₄, filtered and concentrated under vacuum to give intermediate I-08a (500 mg, 69%) as a yellow solid. ESI-MS (M+1): 441.2 calc. for C21H₂₆Cl₂N₂O₄: 440.1.

### Preparation of Intermediate I-09a: 2,4-dichloro-6-methoxy-7-(4-piperidylmethoxy)quinoline

A mixture of intermediate I-08a (500 mg, 1.17 mmol) and HCl/EtOAc (20 mL, 2.0 M) was stirred at 20° C. for 4 hours under N₂. Then, the mixture was concentrated under reduced pressure at 40° C. to give intermediate I-09a (500 mg, crude) as a yellow solid. ESI-MS (M+1): 341.1 calc. for C₁₆H₁₈Cl₂N₂O₂: 340.1.

### Preparation of Intermediate I-10a: 2,4-dichloro-6-methoxy-7-[(1-methyl-4-piperidyl)methoxy]quinoline

To a solution of intermediate I-09a (500 mg, 1.47 mmol) in MeOH (30 mL) were added HCOOH (70 mg, 1.47 mmol), (HCHO)ₙ (R-05a, 396 mg, 4.40 mmol) and NaBH₃CN (276 mg, 4.40 mmol) at 20° C. under N₂ and the mixture was stirred at 60° C. for 16 hours. Then, the mixture was cooled to 20° C. and concentrated under reduced pressure at 40° C. The residue was poured into water and extracted with ethyl acetate. The combined organic phase was washed with brine, dried with anhydrous Na₂SO₄, filtered and concentrated in vacuum to give intermediate I-10a (600 mg, crude) as a yellow solid. ESI-MS (M+1): 355.4 calc. for C₁₇H₂₀Cl₂N₂O₂: 354.1.

### Preparation of Intermediate I-11 a: 4-chloro-6-methoxy-2-(5-methyl-2-furyl)-7-[(1-methyl-4-piperidyl)methoxy]quinoline

To a solution of intermediate I-10a (100 mg, 0.28 mmol) in 1,4-dioxane/H₂O (10:2, 12 mL) were added K₂CO₃ (78 mg, 0.56 mmol), 4,4,5,5-tetramethyl-2-(5-methyl-2-furyl)-1,3,2-dioxaborolane (R-02a, 35 mg, 0.169 mmol) and Pd(PPh₃)₄(33 mg, 0.028 mmol) at 20° C. under N₂ and the mixture was stirred at 110° C. for 16 hours. Then, the mixture was concentrated under reduced pressure at 40° C. to give a residue which was diluted with water and extracted with ethyl acetate. The combined organic phase was washed with brine, dried with anhydrous Na₂SO₄, filtered and concentrated in vacuum to give the crude product which was purified by prep-HPLC (General Procedure, Method 18) to afford intermediate I-11a (50 mg, 73%) as yellow solid. ESI-MS (M+1): 401.2 calc. for C₂₂H₂₅ClN₂O₃: 400.2.

### Preparation of Compound I-08: 6-methoxy-N-methyl-N-(7-methyl-7-azaspiro[3.5]nonan-2-yl)-2-(5-methyl-2-furyl)-7-[(1-methyl-4-piperidyl)methoxy]quinolin-4-amine

To a solution of intermediate I-11a (50 mg, 0.124 mmol) in t-BuOH (5 mL) were added PTSA (32 mg, 0.187 mmol) and N,7-dimethyl-7-azaspiro[3.5]nonan-2-amine (R-03d, 31 mg, 0.187 mmol) at 20° C. under N₂ and the reaction mixture was stirred at 110° C. for 16 hours. Then, the mixture was concentrated under vacuum at 40° C. to give the crude product which was further purified by prep-HPLC (General Procedure, Method 10) to afford compound I-08 (10.8 mg, 16%) as a yellow solid. ESI-MS (M+1): 533.4 calc. for C₃₂H₄₄N₄O₃: 532.3. HPLC analytical method 2, Rt=1.61 min.

Following the same synthetic route for compound I-08 and using the same reagents and intermediates unless otherwise indicated in the table below, the following compounds were obtained:

| | Method | | Method | | |
|---|---|---|---|---|---|
| | Prep- | | Analytical | | |
| Example | HPLC | [M + 1]⁺ | HPLC | Rt | Intermediate/Reagent |
| 1-10 | 40 | 519.3 | 3 | 1.66 | I-07a/tert-butyl 6- |
| | | | | | methylsulfonyloxy-2- |
| | | | | | azaspiro[3.3]heptane-2-carboxylate |
| | | | | | (R-04b)/(HCHO)ₙ (R-05a)/2-(5- |
| | | | | | ethyl-2-furyl)-4,4,5,5-tetramethyl- |
| | | | | | 1,3,2-dioxaborolane (R-02b)/N- |
| | | | | | methyl-1-(1-methyl-4- |
| | | | | | piperidyl)methanamine (R-03c) |
| 1-14 | 41 | 559.4 | 3 | 1.66 | I-07a/tert-butyl 6- |
| | | | | | methylsulfonyloxy-2- |
| | | | | | azaspiro[3.3]heptane-2-carboxylate |
| | | | | | (R-04b)/(HCHO)ₙ (R-05a)/ |
| | | | | | 4,4,5,5-tetramethyl-2-(5-methyl-2- |
| | | | | | furyl)-1,3,2-dioxaborolane (R-02a)/ |
| | | | | | 7-isopropyl-N-methyl-7- |
| | | | | | azaspiro[3.5]nonan-2-amine (R- |
| | | | | | 03e) |
| 1-15 | 41 | 584.4 | 3 | 1.80 | I-07a/tert-butyl 6- |
| | | | | | methylsulfonyloxy-2- |
| | | | | | azaspiro[3.3]heptane-2-carboxylate |
| | | | | | (R-04b)/2-chloroacetonitrile (R- |
| | | | | | 12a)/4,4,5,5-tetramethyl-2-(5- |
| | | | | | methyl-2-furyl)-1,3,2-dioxaborolane |
| | | | | | (R-02a)/7-isopropyl-N-methyl-7- |
| | | | | | azaspiro[3.5]nonan-2-amine (R- |
| | | | | | 03e) |
| 1-16 | 42 | 521.4 | 3 | 1.70 | I-07a/tert-butyl 4- |
| | | | | | (methylsulfonyloxymethyl)piperidine- |
| | | | | | 1-carboxylate (R-04a)/(HCHO)ₙ |
| | | | | | (R-05a)/2-(5-ethyl-2-furyl)-4,4,5,5- |
| | | | | | tetramethyl-1,3,2-dioxaborolane |
| | | | | | (R-02b)/N-methyl-1-(1-methyl-4- |
| | | | | | piperidyl)methanamine (R-03c) |
| 1-20 | 33 | 525.3 | 3 | 1.58 | I-07a/3-(3-fluoropyrrolidin-1- |
| | | | | | yl)propan-1-ol (R-13a)/ |
| | | | | | 4,4,5,5-tetramethyl-2-(5-methyl-2- |
| | | | | | furyl)-1,3,2-dioxaborolane (R-02a)/ |
| | | | | | N-methyl-1-(1-methyl-4- |
| | | | | | piperidyl)methanamine (R-03c) |

Synthetic Route 5

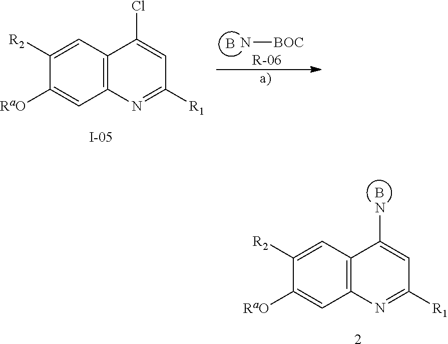

Conditions: a) R-06 (1.2-1.5 eq), PTSA (1.2-2.0 eq), t-BuOH, 120-130° C., 12-48 h.

In the scheme above R₂ is O(C₁-C₆)alkyl, R^{a} is a hydrocarbon chain which contains nitrogen and/or oxygen atoms, R₁ is aryl or heteroaryl and B is as previously defined.

### Preparation of Compound 2-01: 1-[6-methoxy-2-(5-methyl-2-furyl)-7-(3-pyrrolidin-1-ylpropoxy)-4-quinolyl]-N-methyl-piperidin-4-amine

A mixture of intermediate I-05a (300 mg, 0.75 mmol), commercially available tert-butyl 4-(methylamino)piperidine-1-carboxylate (R-06a, 192 mg, 0.9 mmol) and PTSA (155 mg, 0.9 mmol) in t-BuOH (7.5 mL) was degassed and purged with N₂ for 3 times, and then the mixture was stirred at 120° C. for 48 hours under N₂ atmosphere. Then, the reaction mixture was concentrated in vacuum to give crude product which was purified by prep-HPLC (General Procedure, Method 11) to give compound 2-01 (61.1 mg, 17%) as yellow oil. ESI-MS (M+1): 479.3 calc. for C₂₈H₃₈N₄O₃: 478.3. HPLC analytical method 2, Rt=1.31 min.

Following the same synthetic route for compound 2-01 and using the same reagents and intermediates unless otherwise indicated in the table below, the following compounds were obtained:

| | Method | | Method | | |
|---|---|---|---|---|---|
| | Prep- | | Analytical | | |
| Example | HPLC | [M + 1]⁺ | HPLC | Rt | Intermediate/Reagent |
| 2-02 | 12 | 480.3 | 2 | 1.76 | I-05a/tert-butyl 4-(hydroxymethyl)- |
| | | | | | piperidine-1-carboxylate (R-06b) |
| 2-03 | 9 | 478.3 | 2 | 1.44 | I-05d/tert-butyl 4-(methylamino)- |
| | | | | | piperidine-1-carboxylate (R-06a) |
| 2-04 | 13 | 515.3 | 2 | 1.49 | I-05e/tert-butyl 4-(methylamino)- |
| | | | | | piperidine-1-carboxylate (R-06a) |
| 2-06 | 16 | 519.4 | 3 | 1.37 | I-05a/tert-butyl 3-(methylamino)-7- |
| | | | | | azaspiro[3.5]nonane-7-carboxylate |
| | | | | | (R-06c) |
| 2-07 | 7 | 507.3 | 3 | 1.60 | I-05a/tert-butyl 4-(1-amino-1- |
| | | | | | methyl-ethyl)piperidine-1- |
| | | | | | carboxylate (R-06d) |
| 2-09 | 10 | 489.3 | 3 | 1.36 | I-05g/tert-butyl 4- |
| | | | | | (methylaminomethyl)piperidine-1- |
| | | | | | carboxylate (R-06f) |

### Preparation of Compound 2-05: 1-[6-methoxy-2-(5-methyl-1H-pyrrol-2-yl)-7-(3-pyrrolidin-1-ylpropoxy)-4-quinolyl]-N,N-dimethyl-piperidin-4-amine

To a solution of compound 2-03 (10 mg, 0.021 mmol) in MeOH (3 mL) were added HCOOH (1 mg, 0.021 mmol), NaBH₃CN (4 mg, 0.063 mmol) and (HCHO)ₙ (5 mg, 0.063 mmol) at 20° C. under N₂ and the mixture was stirred at 60° C. for 2 hours. Then, the reaction mixture was concentrated under vacuum and the residue was purified by prep-HPLC (General Procedure, Method 14) to afford compound 2-05 (3.40 mg, 32%) as a yellow solid. ESI-MS (M+1): 492.3 calc. for C₂₉H₄₁N₅O₂: 491.3. HPLC analytical method 2, Rt=1.35 min.

### Preparation of Compound 2-08: 1-[1-[2-cyclohexyl-6-methoxy-7-(3-pyrrolidin-1-ylpropoxy)-4-quinolyl]-4-piperidyl]-N-methyl-methanamine

A mixture of intermediate I-05f (500 mg, 1.25 mmol), tert-butyl 4-(methylaminomethyl)piperidine-1-carboxylate (R-06f, 428 mg, 1.88 mmol), PTSA (322 mg, 1.88 mmol) in n-BuOH (30 mL) was degassed and purged with N₂ for 3 times, and then the mixture was stirred at 130° C. for 16 hours under N₂ atmosphere. The mixture was cooled to 20° C. and concentrated in reduced pressure at 40° C. to give the crude product which was purified by prep-HPLC (General Procedure, Method 36) to afford intermediate compound 1-[1-[2-(cyclohexen-1-yl)-6-methoxy-7-(3-pyrrolidin-1-ylpropoxy)-4-quinolyl]-4-piperidyl]-N-methyl-methanamine, which was concentrated under vacuum and freeze-drying (60 mg, 121.78 μmol, 9.74% yield) as yellow oil. ESI-MS (M+1): 493.4 calc. for C₃₀H₄₄N₄O₂: 492.3. To a solution of this intermediate (60 mg, 121.78 μmol) in MeOH (10 mL) was added Pd/C (10%, 10 mg) under H₂ atmosphere. The suspension was degassed and purged with H₂ for 3 times. The mixture was stirred under H₂ (30 Psi) at 20° C. for 2 hours. The mixture was filtered and concentrated in vacuum to give crude product, which was purified by prep-HPLC (General Procedure, Method 35) to give compound 2-08 (23.3 mg, 38.7%) as a yellow syrup. ESI-MS (M+1): 495.5 calc. for C₃₀H₄₆N₄O₂: 494.4. HPLC analytical method 3, Rt=1.52 min.

Synthetic Route 6

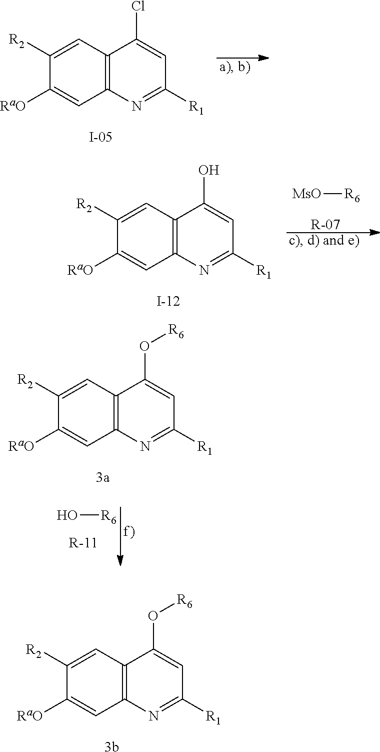

Conditions: a) 4,4,5,5-tetramethyl-2-(4,4,5,5-tetramethyl-1,3,2-dioxaborolan-2-yl)-1,3,2-dioxaborolane (1.1 eq), KOAc (2.5 eq), Pd(dppf)Cl₂ (0.2 eq), 1,4-dioxane, 110° C., 16 h; b) H₂O₂, DCM, 15° C., 16 h; c) R-07a (1.5-2.0 eq), Cs₂CO₃ (2.0-3.0 eq), DMF, 80-120° C., 12-16 h; d) HCl/EtOAc or HCl/MeOH (2.0 M), 15-40° C., 5-12 h; e) (HCHO)ₙ (2.0-3.0 eq), NaBH(OAc)₃ or NaBH₃CN (2.5-3.0 eq), HCOOH (0.1-1.0 eq), MeOH, 40-70° C., 12-16 h; f) PTSA (1.5-3.0 eq), R-11, 120° C., 12 h.

In the scheme above R₂ is O(C₁-C₆)alkyl, R^{a} is a hydrocarbon chain which contains nitrogen and/or oxygen atoms, R₁ is aryl or heteroaryl, and R₆ a cycle (Cy) or a hydrocarbon chain which optionally contains nitrogen oxygen and/or fluor atoms. Conversion f) above can also be performed onto intermediate I-11 to give an analogous compound 3b.

### Preparation of Intermediate I-12a: 6-methoxy-2-(5-methyl-2-furyl)-7-(3-pyrrolidin-1-ylpropoxy)quinolin-4-ol

A mixture of intermediate I-05a (300 mg, 0.75 mmol), 4,4,5,5-tetramethyl-2-(4,4,5,5-tetramethyl-1,3,2-dioxaborolan-2-yl)-1,3,2-dioxaborolane (209 mg, 0.82 mmol), KOAc (183 mg, 1.87 mmol) and Pd(dppf)Cl₂ (109 mg, 0.15 mmol) in 1,4-dioxane (20 mL) was degassed and purged with N₂ for 3 times, and then the mixture was stirred at 100° C. for 16 hours under N₂ atmosphere. Then, the reaction mixture was concentrated in vacuum to give a residue which was purified by silica gel column chromatography to give intermediate 6-methoxy-2-(5-methyl-2-furyl)-7-(3-pyrrolidin-1-ylpropoxy)-4-(4,4,5,5-tetramethyl-1,3,2-dioxaborolan-2-yl)quinoline (350 mg, 95%) as a black solid. To a solution of this intermediate (350 mg, 0.71 mmol) in DCM (15 mL) was added H₂O₂(241 mg, 2.13 mmol) and the mixture was stirred at 15° C. for 16 hours. Then, the reaction mixture was poured into 20 mL water and extracted with DCM. The organic layers was washed with saturated aqueous Na₂SO₃ solution, checked by potassium iodide-starch test paper, dried over Na₂SO₄ and concentrated in vacuum to give crude intermediate I-12a (300 mg, crude) which was used into the next step without further purification as a black solid. ESI-MS (M+1): 383.3 calc. for C₂₂H₂₆N₂O₄: 382.2.

### Preparation of Compound 3-01: 6-methoxy-2-(5-methyl-2-furyl)-4-[(1-methyl-4-piperidyl)oxy]-7-(3-pyrrolidin-1-ylpropoxy)quinoline

To a solution of intermediate I-12a (300 mg, 0.78 mmol) and commercially available tert-butyl 4-methylsulfonyloxypiperidine-1-carboxylate (R-07a, 328 mg, 1.18 mmol) in DMF (10 mL) was added Cs₂CO₃ (766 mg, 2.35 mmol) and the mixture was stirred at 80° C. for 16 hours. Then, the reaction mixture was concentrated in vacuum to give a residue which was purified by TLC to give intermediate tert-butyl 4-[[6-methoxy-2-(5-methyl-2-furyl)-7-(3-pyrrolidin-1-ylpropoxy)-4-quinolyl]oxy]piperidine-1-carboxylate (120 mg, 27%) as a yellow solid. Then, this intermediate (120 mg, 0.21 mmol) was dissolved in HCl/EtOAc (2.0 M, 20 mL) and stirred at 15° C. for 5 hours. Then, the reaction mixture was concentrated in vacuum to give crude product 6-methoxy-2-(5-methyl-2-furyl)-4-(4-piperidyloxy)-7-(3-pyrrolidin-1-ylpropoxy)quinoline (100 mg, crude) as a yellow solid. Finally this intermediate (50 mg, 0.107 mmol) was dissolved in MeOH (5 mL) and (HCHO)ₙ (29 mg, 0.32 mmol), NaBH(OAc)₃ (68 mg, 0.32 mmol) and HCOOH (5 mg, 0.107 mmol) were added under N₂ atmosphere. The mixture was stirred at 70° C. for 16 hours.

Then, the reaction mixture was concentrated in vacuum to give a residue which was purified by prep-HPLC (General procedure, Method 15) to afford compound 3-01 (26.3 mg, 51%) as a yellow solid. ESI-MS (M+1): 480.4 calc. for C₂₈H₃₇N₃O₄: 479.3.

Following the same synthetic route for compound 3-01 and using the same reagents and intermediates unless otherwise indicated in the table below, the following compounds were obtained:

| | Method | | Method | | |
|---|---|---|---|---|---|
| | Prep- | | Analytical | | |
| Example | HPLC | [M + 1]⁺ | HPLC | Rt | Intermediate/Reagent |
| 3-02 | 16 | 494.4 | 2 | 1.32 | I-05a/tert-butyl 4-(methylsulfonyloxy- |
| | | | | | ethyl)piperidine-1-carboxylate (R-07b) |
| 3-03 | 17 | 493.3 | 1 | 2.75 | I-05d/tert-butyl 4-(methylsulfonyloxy- |
| | | | | | methyl)piperidine-1-carboxylate (R-07b) |

### Preparation of Compound 3-04: 7-(3-(pyrrolidin-1-yl)propoxy)-4-butoxy-6-methoxy-2-(5-methylfuran-2-yl)quinoline

A mixture of intermediate I-05a (50 mg, 0.125 mmol) and PTSA (64.43 mg, 0.374 mmol) in n-BuOH (R-11a, 10 mL) was degassed and purged with N₂ for 3 times, and then the mixture was stirred at 120° C. for 12 hours under N₂ atmosphere. Then, the reaction mixture was concentrated under vacuum and purified by prep-HPLC (General Procedure, Method 18) to give compound 3-04 (4.70 mg, 8%) as a yellow solid. ESI-MS (M+1): 439.3 calc. for C₂₆H₃₄N₂O₄: 438.3. HPLC analytical method 2, Rt=2.01 min.

Following the same synthetic route for compound 3-04 and using the same reagents and intermediates unless otherwise indicated in the table below, the following compounds were obtained:

| | Method | | Method | | |
|---|---|---|---|---|---|
| | Prep- | | Analytical | | |
| Example | HPLC | [M + 1]⁺ | HPLC | Rt | Intermediate |
| 3-05 | 18 | 475.3 | 2 | 2.40 | I-05e |
| 3-07 | 28 | 457.3 | 3 | 2.38 | I-05h (4-chloro-7-[3-(3- |
| | | | | | fluoropyrrolidin-1-yl)propoxy]-6- |
| | | | | | methoxy-2-(5-methyl-2- |
| | | | | | furyl)quinoline); this intermediate |
| | | | | | was prepared by synthetic route 4 |
| | | | | | starting from intermediate I-07a |
| | | | | | and by reacting it firstly with |
| | | | | | 4,4,5,5-tetramethyl-2-(5-methyl-2- |
| | | | | | furyl)-1,3,2-dioxaborolane (R-02a) |
| | | | | | (step f), and then reacting the |
| | | | | | obtained intermediate with 3-(3- |
| | | | | | fluoropyrrolidin-1-yl)propan-1-ol |
| | | | | | (R-13a) (step e). |

Synthetic Route 7

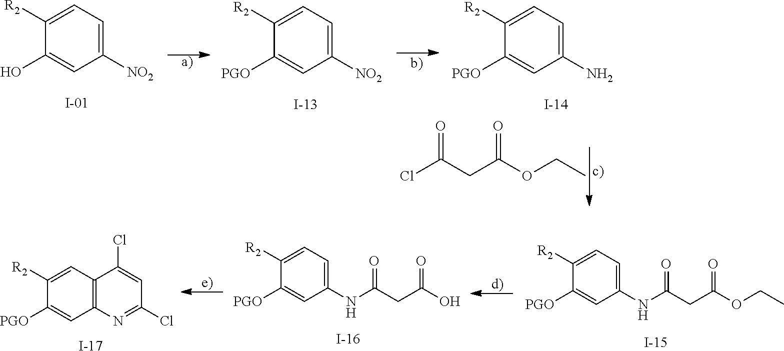

Conditions: a) BrBn (1.5 eq), CH₃CN, 15° C., 40 h; b) NH₄Cl (3.0 eq), Fe (8.0 eq), EtOH/H₂O (1:1), 100° C., 2 h; c) pyridine (3.5 eq), DMAP (0.1 eq), 3-chloro-3-oxo-propanoate (0.9 eq), DCM, −78° C., then 18° C., 2 h; d) NaOH (2.0 eq), THF/MeOH/H₂O (10:3:3), 18° C., 5 h; e) POCl₃, 100° C., 2 h.

In the scheme above R₂ is O(C₁-C₆)alkyl and PG is a protective group.

### Preparation of Intermediate I-13a: 2-benzyloxy-1-methoxy-4-nitro-benzene

A mixture of 2-methoxy-5-nitro-phenol (I-01a, 300 g, 1.77 mmol) and BrBn (454.08 g, 2.66 mmol) in CH₃CN (3 L) was stirred at 15° C. for 40 hours. Then, the residue was poured into water (15 L) and filtered through a Celite pad. The filter cake was concentrated in vacuum to give intermediate I-13a (400 g, 87%) as brown solid. ESI-MS (M+1): 260.1 calc. for C₁₄H₁₃NO₄: 259.1.

### Preparation of Intermediate I-14a: 3-benzyloxy-4-methoxy-aniline

To a solution of intermediate I-13a (200 g, 771 mmol) in EtOH/H₂O (2 L, 1:1) were added NH₄Cl (123.79 g, 2.31 mol) and Fe (344.67 g, 6.17 mol) in batches and the mixture was stirred at 100° C. for 2 hours. Then, the mixture was filtered through a Celite pad. The filtrate was concentrated in vacuum to give a residue which was diluted with 1 L water and 1 L EtOAc. Organic phase was separated and aqueous phase was extracted with EtOAc. The combined organic layers were dried over Na₂SO₄ and concentrated in vacuum to give intermediate I-14a (166 g, 94%) as yellow solid which was used in the next step without further purification.

### Preparation of Intermediate I-15a: ethyl 3-(3-benzyloxy-4-methoxy-anilino)-3-oxo-propanoate

To a solution of intermediate I-14a (166 g, 724 mmol) in DCM (2 L) were added pyridine (200 g, 2.53 mol) and DMAP (8.85 g, 72.40 mmol) at 18° C. under N₂. Then ethyl 3-chloro-3-oxo-propanoate (98.11 g, 651.64 mmol) in DCM (100 mL) was added drop wise to the above solution at −78° C. and the mixture was stirred at 18° C. for 2 hours. Then, the residue was poured into water (1 L). The aqueous phase was extracted with DCM and the combined organic phase was washed with brine twice, dried with anhydrous Na₂SO₄, filtered and concentrated in vacuum to give intermediate I-15a (250 g, crude) as red solid. ESI-MS (M+1): 344.2 calc. for C₁₉H₂₁NO₅: 343.1.

### Preparation of Intermediate I-16a: 3-(3-benzyloxy-4-methoxy-anilino)-3-oxo-propanoic acid

To a solution of intermediate I-15a (250 g, 728 mmol) in THF/MeOH/H₂O (1.6 L, 10:3:3) was added NaOH (58.25 g, 1.46 mol) and the mixture was stirred at 18° C. for 5 hours. Then, the reaction mixture was concentrated under reduced pressure to remove the organic phase. The residue was poured into water and the pH was adjusted to 5 by progressively adding aqueous 2 M HCl. The solution was filtered and the filter cake was dried in air to give intermediate I-16a (165 g, 72%) as yellow solid. ESI-MS (M+1): 316.2 calc. for C₁₇H₁₇NO₅: 315.1.

### Preparation of Intermediate I-17a: 7-benzyloxy-2,4-dichloro-6-methoxy-quinoline

Intermediate I-16a (56 g, 177 mmol) was added slowly into POCl₃ (811 g, 5.29 mol) in batches at 18° C. and the resulting mixture was stirred at 100° C. for 2 hours. Then, the solution was concentrated under reduced pressure at 45° C. and the residue was poured into cold water and stirred for 10 minutes. The aqueous phase was extracted with DCM and the combined organic phase was washed with brine, dried with anhydrous Na₂SO₄, filtered and concentrated in vacuum. The residue was purified by column chromatography to give the pure intermediate I-17a (8 g, 14%) as a white solid. ESI-MS (M+1): 334.1 calc. for C₁₇H₁₃Cl₂NO₂: 333.0.

Synthetic Route 8

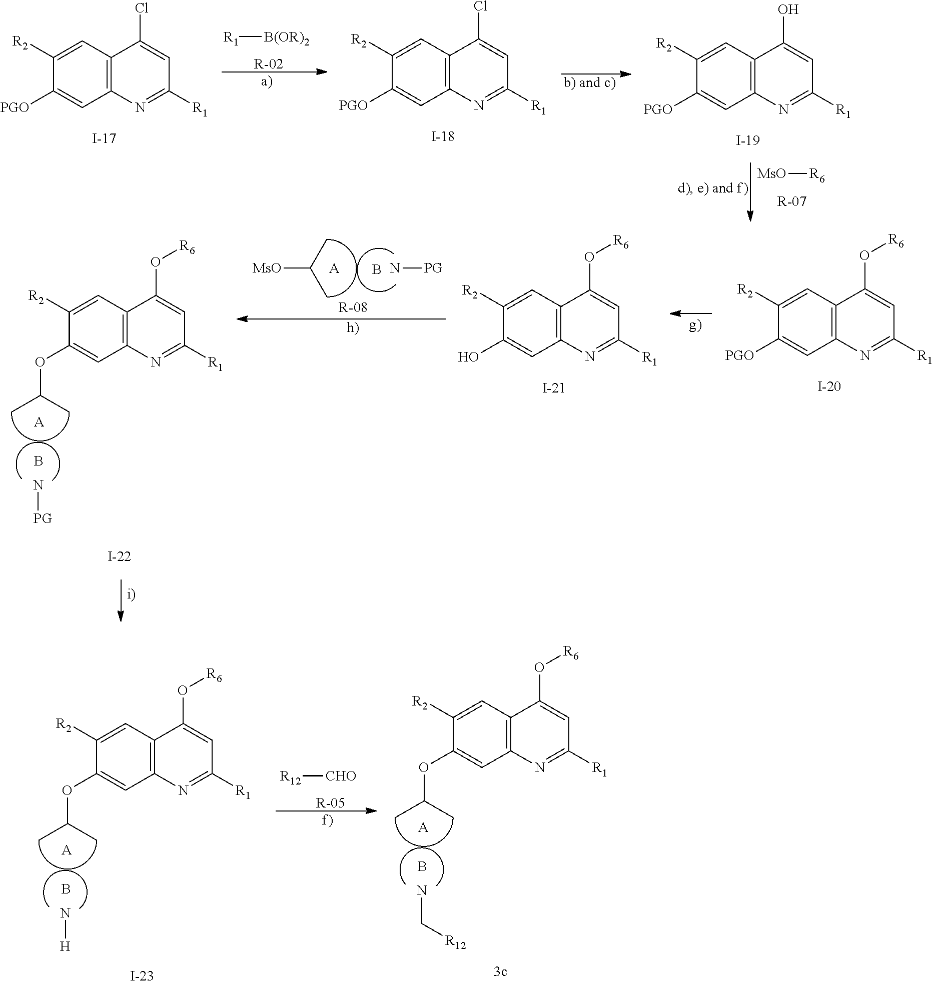

Conditions: a) R-02 (1.1 eq), K₂CO₃ (2.0 eq), Pd(PPh₃)₄(0.1 eq), 1,4-dioxane/H₂O (1:1), 110° C., 16 h; b) 4,4,5,5-tetramethyl-2-(4,4,5,5-tetramethyl-1,3,2-dioxaborolan-2-yl)-1,3,2-dioxaborolane (1.1 eq), KOAc (3.0 eq), Pd(dppf)Cl₂ (0.1 eq), 1,4-dioxane, 100° C., 12 h; c) NaOH (3.0 eq), H₂O₂(3.0 eq), THF/H₂O (20:3), 25° C., 12 h; d) R-07 (1.5 eq), Cs₂CO₃ (2.0 eq), DMF, 120° C., 12 h; e), HCl/MeOH (2.0 M), 25° C., 12 h; f) (HCHO)ₙ (2.0-3.0 eq), NaBH₃CN (2.5-3.0 eq), HCOOH (0.1-1.0 eq), MeOH, 40-60° C., 12-16 h; g) Pd/C, MeOH, H₂ (15 Psi), 25° C., 6 h; h) R-08 (1.5 eq), Cs₂CO₃ (2.0 eq), DMF, 100° C., 16 h; i) TFA (1.0 eq), DCM, 20° C., 5 h.

In the scheme above R₂ is O(C₁-C₆)alkyl, R₁ is aryl or heteroaryl, R is H, (C₁-C₆)alkyl or, alternatively, two R groups together with the B atom to which they are attached may form a cycle, R₆ a cycle (Cy) or a hydrocarbon chain which optionally contains nitrogen oxygen and/or fluor atoms, R₁₂ is H or (C₁-C₆)alkyl and PG is a protective group.

### Preparation of Intermediate I-18a: 7-benzyloxy-4-chloro-6-methoxy-2-(5-methyl-2-furyl)quinoline

To a solution of intermediate I-17a (8 g, 24 mmol) in 1,4-dioxane/H₂O (300 mL, 1:1) were added K₂CO₃ (6.62 g, 48 mmol), Pd(PPh₃)₄(2.77 g, 2.39 mmol) and 4,4,5,5-tetramethyl-2-(5-methyl-2-furyl)-1,3,2-dioxaborolane (R-02a, 5.98 g, 28.73 mmol) at 18° C. and the resulting mixture was degassed and purged with N₂ for 3 times. The mixture was stirred at 110° C. for 16 hours under N₂ and then concentrated in reduced pressure at 45° C. The residue was poured into water and the aqueous phase was extracted with EtOAc. The combined organic phase was washed with brine, dried with anhydrous Na₂SO₄, filtered and concentrated in vacuum. The residue was purified by column chromatography to give pure intermediate I-18a (6.7 g, 74%) as a brown solid. ESI-MS (M+1): 380.2 calc. for C₂₂H₁₈ClNO₃: 379.1.

### Preparation of Intermediate I-19a: 7-benzyloxy-6-methoxy-2-(5-methyl-2-furyl)quinolin-4-ol

A mixture of intermediate I-18a (500 mg, 1.32 mmol), 4,4,5,5-tetramethyl-2-(4,4,5,5-tetramethyl-1,3,2-dioxaborolan-2-yl)-1,3,2-dioxaborolane (401 mg, 1.58 mmol), KOAc (387 mg, 3.95 mmol) and Pd(dppf)Cl₂ (96 mg, 0.13 mmol) in 1,4-dioxane (30 mL) was degassed and purged with N₂ for 3 times, and then the mixture was stirred at 100° C. for 12 hours under N₂ atmosphere. Then, the reaction mixture was filtrated and the filtrate was concentrated under vacuum to give intermediate 7-benzyloxy-6-methoxy-2-(5-methyl-2-furyl)-4-(4,4,5,5-tetramethyl-1,3,2-dioxaborolan-2-yl)quinoline (500 mg, 80%) as a black solid. A mixture of this intermediate (500 mg, 1.06 mmol), NaOH (127 mg, 3.18 mmol) and H₂O₂(108 mg, 3.18 mmol) in THF/H₂O (23 mL, 20:3) was degassed and purged with N₂ for 3 times, and then the mixture was stirred at 25° C. for 12 hours under N₂ atmospheres. Then, the reaction mixture was quenched by addition of Na₂SO₃ (2 g), stirred for additional 30 minutes, followed by addition of HCl (2 M, 5 mL) and then extracted with EtOAc. The combined organic layers were dried over Na₂SO₄, filtered and concentrated under reduced pressure to give intermediate I-19a (250 mg, 65%) as a yellow solid. ESI-MS (M+1): 362.2 calc. for C₂₂H₁₉NO₄: 361.1.

### Preparation of Intermediate I-20a: 7-benzyloxy-6-methoxy-2-(5-methyl-2-furyl)-4-[(1-methyl-4-piperidyl)oxy]quinoline

A mixture of intermediate I-19a (500 mg, 1.38 mmol), commercially available tert-butyl 4-methylsulfonyloxypiperidine-1-carboxylate (R-07a, 580 mg, 2.08 mmol) and Cs₂CO₃ (902 mg, 2.77 mmol) in DMF (5.00 mL) was degassed and purged with N₂ for 3 times. The mixture was stirred at 120° C. for 12 hours under N₂ atmosphere. Then, the reaction mixture was filtrated and the filtrate was concentrated under vacuum to give intermediate tert-butyl 4-[[7-benzyloxy-6-methoxy-2-(5-methyl-2-furyl)-4-quinolyl]oxy]piperidine-1-carboxylate (500 mg, crude) as a yellow solid which was used in the next step without further purification. Then, a mixture of this intermediate (500 mg, 0.918 mmol) in HCl/MeOH (20.00 mL, 2 M) was degassed and purged with N₂ for 3 times and the mixture was stirred at 25° C. for 12 hours under N₂ atmosphere. Then, the reaction mixture was concentrated under vacuum to give intermediate 7-benzyloxy-6-methoxy-2-(5-methyl-2-furyl)-4-(4-piperidyloxy)quinoline (400 mg, 98%) as a yellow solid. Finally, a mixture of this intermediate (500 mg, 1.12 mmol), (HCHO)ₙ (202 mg, 2.24 mmol), HCOOH (5.38 mg, 0.112 mmol) and NaBH₃CN (176 mg, 2.80 mmol) in MeOH (10 mL) was degassed and purged with N₂ for 3 times, and then the mixture was stirred at 40° C. for 12 hours under N₂ atmosphere. Then, the reaction mixture was concentrated under vacuum to give the crude product which was purified by prep-HPLC (General Procedure, Method 19) to give intermediate I-20a (260 mg, 51%) as a yellow solid. ESI-MS (M+1): 459.3 calc. for C₂₈H₃₀N₂O₄: 458.2.

### Preparation of Intermediate I-21a: 6-methoxy-2-(5-methyl-2-furyl)-4-[(1-methyl-4-piperidyl)oxy]quinolin-7-ol

A mixture of intermediate I-20a (250 mg, 0.54 mmol) and Pd/C (20 mg) in MeOH (10 mL) was degassed and purged with N₂ for 3 times. Then, the mixture was stirred at 25° C. for 6 hours under H₂ atmosphere (15 Psi). The reaction mixture was filtrated and the filtrate was concentrated under vacuum to give intermediate I-21a (190 mg, 945%) as a white solid. ESI-MS (M+1): 369.2 calc. for C₂₁H₂₄N₂O₄: 368.2.

### Preparation of Intermediate I-22a: tert-butyl 6-[[6-methoxy-2-(5-methyl-2-furyl)-4-[(1-methyl-4-piperidyl)oxy]-7-quinolyl]oxy]-2-azaspiro[3.3]heptane-2-carboxylate

To a solution of intermediate I-21a (200 mg, 0.54 mmol) in DMF (15 mL) were added Cs₂CO₃ (354 mg, 1.09 mmol) and tert-butyl 6-methylsulfonyloxy-2-azaspiro[3.3]heptane-2-carboxylate (R-08a, 238 mg, 0.81 mmol) at 20° C. under N₂ and the mixture was stirred at 100° C. for 16 hours. Then, the reaction mixture was filtered and concentrated under reduced pressure to afford intermediate I-22a (450 mg, crude) as a yellow solid. ESI-MS (M+1): 564.4 calc. for C₃₂H₄₁N₃O₆: 563.3.

### Preparation of Intermediate I-23a: 7-(2-azaspiro[3.3]heptan-6-yloxy)-6-methoxy-2-(5-methyl-2-furyl)-4-[(1-methyl-4-piperidyl)oxy]quinoline

To a solution of intermediate I-22a (300 mg, 0.53 mmol) in DCM (6 mL) were added TFA (61 mg, 0.53 mmol, 39.40 μL) at 0° C. and the mixture was stirred at 20° C. for 5 hours. Then, the reaction mixture was concentrated under reduced pressure to afford intermediate I-23a (650 mg, crude) as a yellow solid. ESI-MS (M+1): 464.3 calc. for C₂₇H₃₃N₃O₄: 463.3.

### Preparation of Compound 3-06: 6-methoxy-7-[(2-methyl-2-azaspiro[3.3]heptan-6-yl)oxy]-2-(5-methyl-2-furyl)-4-[(1-methyl-4-piperidyl)oxy]quinoline

To a solution of intermediate I-23a (650 mg, 1.40 mmol) in MeOH (10 mL) were added HCOOH (68 mg, 1.40 mmol), NaBH₃CN (265 mg, 4.21 mmol) and (HCHO)ₙ (R-05a, 379 mg, 4.21 mmol) and the reaction mixture was stirred at 60° C. for 16 hours. Then, the reaction was filtered and the filtrate was concentrated under reduced pressure to give a residue which was purified by prep-HPLC (General procedure, Method 20) to afford compound 3-06 (18 mg, 3%). ESI-MS (M+1): 478.3 calc. for C₂₈H₃₅N₃O₄: 477.3. HPLC analytical method 2, Rt=1.33 min.

Synthetic Route 9

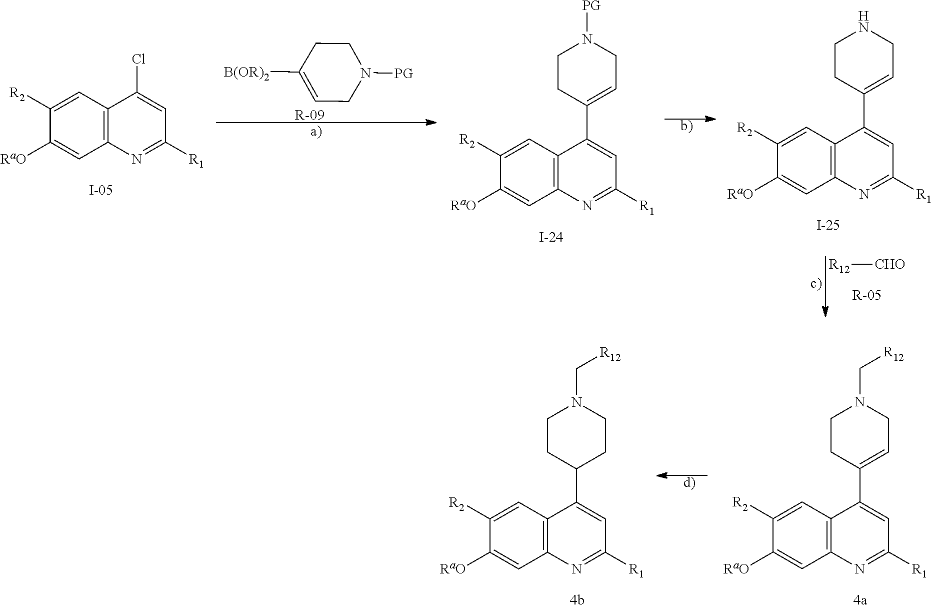

Conditions: a) R-09 (1.5 eq), KOAc (2.0 eq), Pd(PPh₃)₄(0.1 eq), 1,4-dioxane/H₂O (10:1), 120° C., 12 h; b) HCl/MeOH (2.0 M), 25° C., 12 h; c) R-05 (2.0 eq), HCOOH (0.1 eq), NaBH₃CN (2.0 eq), MeOH, 60° C., 12 h; d) Pd/C, MeOH, H₂ (15-50 Psi), 25-60° C., 12-15 h.

In the scheme above R₂ O(C₁-C₆)alkyl, R^{a} is a hydrocarbon chain which contains nitrogen and/or oxygen atoms, R₁ is aryl or heteroaryl, R is H, (C₁-C₆)alkyl or, alternatively, two R groups together with the B atom to which they are attached may form a cycle, R₁₂ is H or (C₁-C₆)alkyl and PG is a protective group.

### Preparation of Intermediate I-24a: tert-butyl 4-[6-methoxy-2-(5-methyl-2-furyl)-7-(3-pyrrolidin-1-ylpropoxy)-4-quinolyl]-3,6-dihydro-2H-pyridine-1-carboxylate

A mixture of I-05a (300 mg, 0.75 mmol), tert-butyl 4-(4,4,5,5-tetramethyl-1,3,2-dioxaborolan-2-yl)-3,6-dihydro-2H-pyridine-1-carboxylate (R-09a, 347 mg, 1.12 mmol), KOAc (147 mg, 1.50 mmol) and Pd(PPh₃)₄(86 mg, 0.075 mmol) in 1,4-dioxane/H₂O (11 mL, 10:1) was degassed and purged with N₂ for 3 times, and then the mixture was stirred at 120° C. for 12 hours under N₂ atmosphere. Then, the reaction mixture was filtrated and the filtrate was concentrated under vacuum to give intermediate I-24a (350 mg, crude) as a yellow solid. ESI-MS (M+1): 548.4 calc. for C₃₂H₄₁N₃O₅: 547.3.

### Preparation of intermediate I-25a: 6-methoxy-2-(5-methyl-2-furyl)-7-(3-pyrrolidin-1-ylpropoxy)-4-(1,2,3,6-tetrahydropyridin-4-yl)quinoline

A mixture of intermediate I-24a (300 mg, 0.55 mmol) and HCl/MeOH (10 mL, 2 M) was degassed and purged with N₂ for 3 times. Then the mixture was stirred at 25° C. for 12 hours under N₂ atmosphere. Then, the reaction mixture was concentrated under vacuum to give intermediate I-25a (220 mg, 89%) as a white solid. ESI-MS (M+1): 448.3 calc. for C₂₇H₃₃N₃O₃: 447.3.

### Preparation of Compound 4-01: 6-methoxy-4-(1-methyl-3,6-dihydro-2H-pyridin-4-yl)-2-(5-methyl-2-furyl)-7-(3-pyrrolidin-1-ylpropoxy)quinoline

A mixture of intermediate I-25a (220 mg, 0.49 mmol), (HCHO)ₙ (R-05a, 89 mg, 0.98 mmol), HCOOH (2.36 mg, 0.049 mmol) and NaBH₃CN (62 mg, 0.98 mmol) in MeOH (20 mL) was degassed and purged with N₂ for 3 times, then the mixture was stirred at 60° C. for 12 hours under N₂ atmosphere. Then, the reaction mixture was concentrated under vacuum to give a residue which was purified by prep-HPLC (General Procedure, Method 21) to give compound 4-01 (150 mg, 66%) as a yellow solid. ESI-MS (M+1): 462.3 calc. for C₂₈H₃₅N₃O₃: 461.3. HPLC analytical method 2, Rt=1.36 min.

### Preparation of Compound 4-02: 6-methoxy-2-(5-methyl-2-furyl)-4-(1-methyl-4-piperidyl)-7-(3-pyrrolidin-1-ylpropoxy)quinoline

A mixture of compound 4-01 (100 mg, 0.216 mmol) and Pd/C (50 mg) in MeOH (20 mL) was degassed and purged with H₂ for 3 times, then the mixture was stirred at 25° C. for 12 hours under H₂ atmosphere (15 Psi). Then, the reaction mixture was filtrated and the filtrate was concentrated under vacuum to give a residue which was purified by prep-HPLC (General Procedure, Method 22) to give compound 4-02 (40.3 mg, 40%) as a yellow solid. ESI-MS (M+1): 464.3 calc. for C₂₈H₃₇N₃O₃: 463.3. HPLC analytical method 2, Rt=1.28 min.

### Preparation of Compound 4-03: 6-methoxy-4-(1-methyl-4-piperidyl)-2-(5-methyltetrahydrofuran-2-yl)-7-(3-pyrrolidin-1-ylpropoxy)quinoline

A mixture of compound 4-01 (50 mg, 0.11 mmol) and Pd/C (50 mg) in MeOH (20 mL) was degassed and purged with H₂ for 3 times, then the mixture was stirred at 60° C. for 12 hours under H₂ atmosphere (50 Psi). Then, the reaction mixture was filtrated and the filtrate was concentrated under vacuum to give a residue which was purified by prep-HPLC (General Procedure, Method 23) to give compound 4-03 (15 mg, 29%) as a white solid. ESI-MS (M+1): 468.3 calc. for C₂₈H₄₁N₃O₃: 467.3. HPLC analytical method 2, Rt=1.06 min.

Synthetic Route 10

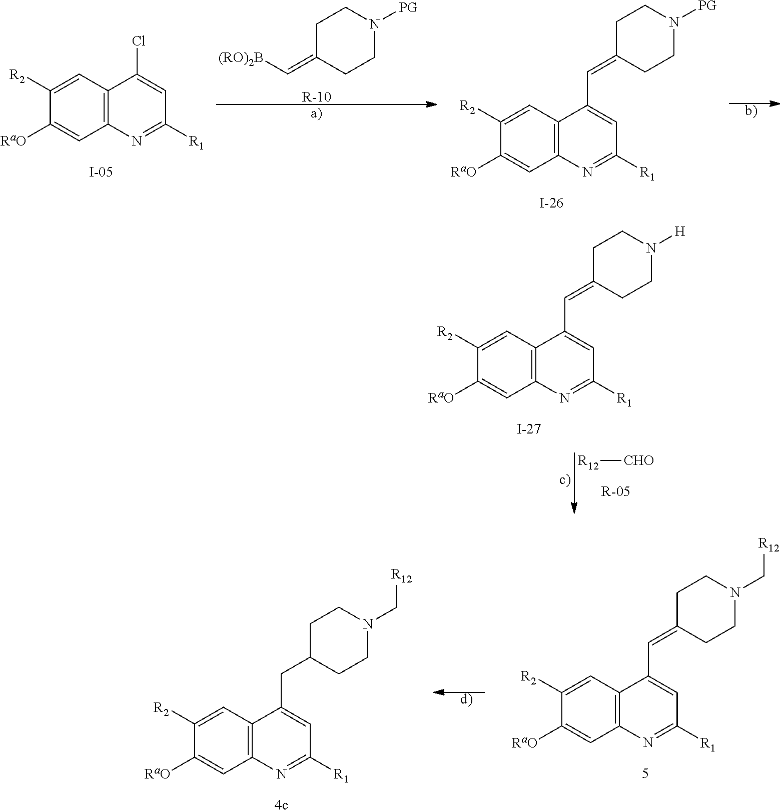

Conditions: a) R-10 (1.0 eq), K₂CO₃ (1.0 eq), Pd(PPh₃)₄(0.1 eq), 1,4-dioxane/H₂O (6:1), 110° C., 16 h; b) HCl/EtOAc (2.0 M), 15° C., 30 minutes; c) R-05 (3.0 eq), HCOOH (1.0 eq), NaBH₃CN (3.0 eq), MeOH, 60° C., 16 h; d) Pd/C, MeOH, H₂ (15 Psi), 20° C., 1 h.

In the scheme above R₂ O(C₁-C₆)alkyl, R^{a} is a hydrocarbon chain which contains nitrogen and/or oxygen atoms, R₁ is aryl or heteroaryl, R is H, (C₁-C₆)alkyl or, alternatively, two R groups together with the B atom to which they are attached may form a cycle, R₁₂ is H or (C₁-C₆)alkyl and PG is a protective group.

### Preparation of Intermediate I-26a: tert-butyl 4-[[6-methoxy-2-(5-methyl-2-furyl)-7-(3-pyrrolidin-1-ylpropoxy)-4-quinolyl]methylene]piperidine-1-carboxylate

To a solution of I-05a (180 mg, 0.449 mmol) in 1,4-dioxane/H₂O (3.5 mL, 6:1) were added Pd(PPh₃)₄(52 mg, 0.045 mmol), tert-butyl 4-[(4,4,5,5-tetramethyl-1,3,2-dioxaborolan-2-yl)methylene]piperidine-1-carboxylate (R-10a, 140 mg, 0.449 mmol) and K₂CO₃ (62 mg, 0.449 mmol) at 20° C. under N₂ atmospheres and the mixture was stirred at 110° C. for 16 hours. Then, the reaction mixture was concentrated under vacuum to give a residue which was poured into water and extracted with ethyl acetate. The combined organic phase was washed with brine, dried with anhydrous Na₂SO₄, filtered and concentrated under vacuum to give crude product which was further purified by prep-HPLC (General Procedure, Method 24) to give intermediate I-26a (120 mg, 48%) as a yellow solid. ESI-MS (M+1): 562.4 calc. for C₃₃H₄₃N₃O₅: 561.3.

### Preparation of Intermediate I-27a: 6-methoxy-2-(5-methyl-2-furyl)-4-(4-piperidylidenemethyl)-7-(3-pyrrolidin-1-ylpropoxy)quinoline

A mixture of intermediate I-26a (100 mg, 0.178 mmol) and HCl/EtOAc (5 mL, 2 M) was stirred at 15° C. for 30 minutes. Then, the reaction mixture was concentrated under reduced pressure at 40° C. to give intermediate I-27a (100 mg, crude) as a yellow solid. ESI-MS (M+1): 462.4 calc. for C₂₈H₃₅N₃O₃: 461.3.

### Preparation of Compound 5-01: 6-methoxy-2-(5-methyl-2-furyl)-4-[(1-methyl-4-piperidyl idene)methyl]-7-(3-pyrrolidin-1-ylpropoxy)quinoline and compound 5-02: 2-[4-[[6-methoxy-2-(5-methyl-2-furyl)-7-(3-pyrrolidin-1-ylpropoxy)-4-quinolyl]methylene]-1-piperidyl]acetonitrile

To a solution of I-27a (100 mg, 0.216 mmol) in MeOH (10 mL) were added HCOOH (10 mg, 0.216 mmol), NaBH₃CN (41 mg, 0.65 mmol) and (HCHO)ₙ (R-05a, 59 mg, 0.65 mmol) at 20° C. under N₂ and the mixture was stirred at 60° C. for 16 hours. Then, the mixture was concentrated under reduced pressure at 40° C. to give a residue was purified prep-HPLC (General Procedure, Method 25) to give Compound 5-01 (100 mg, crude) and compound 5-02 (20.5 mg, 19%) as a yellow solid. 20 mg of crude compound 5-01 was purified again by prep-HPLC (General Procedure, Method 25) to give 5.2 mg qualified sample (25.4% yield) as a yellow solid.

Compound 5-01: ESI-MS (M+1): 476.4 calc. for C₂₉H₃₇N₃O₃: 475.3. HPLC analytical method 2, Rt=1.41 min.

Compound 5-02: ESI-MS (M+1): 501.3 calc. for C₃₀H₃₆N₄O₃: 500.3. HPLC analytical method 2, Rt=1.86 min.

### Preparation of Compound 4-04: 6-methoxy-4-[(1-methyl-4-piperidyl)methyl]-2-(5-methyltetrahydrofuran-2-yl)-7-(3-pyrrolidin-1-ylpropoxy)quinoline

To a solution of compound 5-01 (17 mg, 0.031 mmol) in MeOH (15 mL) was added Pd/C (3 mg) under H₂ atmosphere and the suspension was degassed and purged with H₂ for 3 times. The mixture was stirred under H₂ (15 Psi) at 20° C. for 1 hour. Then, the mixture was filtered and the filtrate was concentrated under reduced pressure at 40° C. to give a residue which was purified by prep-HPLC (General Procedure, Method 26) to give compound 4-04 (2.90 mg, 19%) as a yellow solid. ESI-MS (M+1): 482.4 calc. for C₂₉H₄₃N₃O₃: 481.3. HPLC analytical method 2, Rt=1.20 min.

### Biological Tests

### G9a Enzyme Activity Assay

The biochemical assay to measure G9a enzyme activity relies on time-resolved fluorescence energy transfer (TR-FRET) between europium cryptate (donor) and XL665 (acceptor). TR-FRET is observed when biotinylated histone monomethyl-H3K9 peptide is incubated with cryptate-labeled anti-dimethyl-histone H3K9 antibody (CisBio Cat#61KB2KAE) and streptavidin XL665 (CisBio Cat#610SAXLA), after enzymatic reaction of G9a. The human G9a enzyme expressed in a baculovirus infected Sf9 cell expression system was obtained from BPS Biosciences (Cat. #51001). Enzyme activity assay was carried out in a white 384-well plate in a final volume of 20 μl, as follow:
4 μl of vehicle or studied compound 2.5× concentrated prepared in assay buffer (50 mM Tris-HCl, 10 mM NaCl, 4 mM DTT, 0.01% Tween-20 pH9). Final percentage of DMSO was 0.5%.
2 μl of 1 nM G9a enzyme diluted in assay buffer. Final concentration was 0.2 nM.
Start the reaction by adding 4 μl of substrate mixture containing 20 μM S-adenosylmethionine and 40 nM biotinylated histone monomethyl-H₃K9 peptide.
Reaction was carried out during 1 hour at room temperature.
Enzyme activity was stopped by adding 5 μl of cryptate-labeled anti-dimethyl-histone H₃K9 antibody. Final concentration 150 nM.
Then, add 5 μl of streptavidin XL665 beads. Final concentration of 16 μM.
Read the plate after 1 hour of incubation at room temperature.

For each well, fluorescence was measured at 620 nm and 665 nm. A ratio (665 nm/620 nm) was then calculated in order to minimize medium interferences. Positive control was obtained in the presence of the vehicle of the compounds. Negative control was obtained in the absence of G9a enzyme activity. Calculated IC₅₀ values were determined using GraphPrism using 4-parameters inhibition curve.

DNMT1 Enzyme Activity Assay

The biochemical assay to measure DNMT1 enzyme activity relies on time-resolved fluorescence energy transfer (TR-FRET) between lumi4-Tb (donor) and d2 (acceptor) using the EPIgeneous methyltransferase assay (CisBio Cat#62SAHPEB). TR-FRET is observed when antibody specific to S-adenosylhomocysteine labeled with Lumi4-Tb is incubated with d2-labeled S-adenosylhomocysteine. TR-FRET signal is inversely proportional to the concentration of SAH, product of DNMT1 enzyme activity, in the sample. The human DNMT1 was obtained from Reaction Biology Corp. (Cat# DMT-21-124).

Enzyme activity assay was carried out in a white 384-well plate in a final volume of 20 μl, as follow:
4 μl of vehicle or studied compound 2.5× concentrated prepared in assay buffer (50 mM Tris-HCl, 1 mM EDTA, 1 mM DTT, 0.1% Triton X-100, 5% glycerol pH 7.5). Final percentage of DMSO was 0.5%.
2 μl of 1 nM DNMT1 enzyme diluted in assay buffer. Final concentration was 20 nM.
Start the reaction by adding 4 μl of substrate mixture containing 1 μM S-adenosylmethionine and 1 μM poly-deoxy inosine poly-deoxy cytosine (pdI-pdC) DNA.
Reaction was carried out during 15 minutes at 37° C.
Enzyme activity was stopped by adding 2 μl of buffer one of the EPIgeneous methyltransferase assay.
After 10 minutes at room temperature, it was added 4 μl of antibody specific to S-adenosylhomocysteine labeled with Lumi4-Tb 50× diluted in buffer two of the EPIgeneous methyltransferase assay.
Add 4 μl of d2-labeled S-adenosylhomocysteine 31× diluted in buffer two of the EPIgeneous methyltransferase assay.
Read the plate after 1 hour of incubation at room temperature.

For each well, fluorescence was measured at 620 nm and 665 nm. A ratio (665 nm/620 nm) was then calculated in order to minimize medium interferences. Positive control was obtained in the presence of the vehicle of the compounds. Calculated IC₅₀ values were determined using GraphPrism using 4-parameters inhibition curve.

Table 1 shows the inhibition values for DNMT1 (IC₅₀) and G9a (IC₅₀) for selected compounds; where 1 μM≤IC₅₀≤10 μM (+), 500 nM≤IC₅₀<1 μM (++), 100 nM≤IC₅₀<500 nM (+++), IC₅₀<100 nM (++++) and IC₅₀>10 μM (N.A. not active)

TABLE 1

| | DNMT1 | G9a |
|---|---|---|
| Compound | IC₅₀(M) | IC₅₀ (M) |
| 1-02 | + | ++ |
| 1-03 | +++ | + |
| 1-04 | +++ | ++ |
| 1-05 | + | + |
| 1-06 | +++ | +++ |
| 1-07 | ++ | ++++ |
| 1-08 | + | N.A. |
| 1-09 | ++ | ++ |
| 1-10 | + | N.A. |
| 1-11 | + | +++ |
| 1-12 | + | + |
| 1-14 | + | N.A. |
| 1-15 | + | N.A. |
| 1-16 | + | N.A. |
| 1-18 | + | + |
| 1-19 | ++ | ++ |
| 1-20 | + | N.A. |
| 1-21 | + | +++ |
| 1-22 | ++ | + |
| 1-23 | ++ | +++ |
| 2-01 | ++ | + |
| 2-02 | + | + |
| 2-03 | ++ | +++ |
| 2-04 | + | N.A. |
| 2-05 | +++ | +++ |
| 2-06 | + | ++ |
| 2-07 | + | ++ |
| 2-08 | + | ++++ |
| 2-09 | + | +++ |
| 3-01 | + | + |
| 3-02 | ++ | + |
| 3-03 | ++ | +++ |
| 3-06 | + | N.A. |
| 4-02 | + | + |
| 5-01 | + | N.A. |

Cell Proliferation Assay

Cell proliferation was analyzed after 48 hours of in vitro treatment using the CellTiter 96 Aqueous One Solution Cell Proliferation Assay (Promega, Madison, W). This is a colorimetric method for determining the number of viable cells in proliferation.

For the assay, suspension cells were cultured by triplicate at a density of 1×10⁶ cells/ml in 96-well plates (100.000 cells/well, 100 μl/well), except for HepG2, Hep3B and PLC/PRF/5 cell lines which were cultured at a density of 3000 cells/well, 100 μl/well).

Adherent cells were obtained from 80-90% confluent flasks and 100 μl of cells were seeded at a density of 5000 cells/well in 96-well plates by triplicate. Before addition of the compounds, adherent cells were allowed to attach to the bottom of the wells for 12 hours. In all cases, only the 60 inner wells were used to avoid any border effects.

After 48 hours of treatment, plates with suspension cells were centrifuged at 800g for 10 minutes and medium was removed. The plates with adherent cells were flicked to remove medium. Then, cells were incubated with 100 μl/well of medium and 20 μl/well of CellTiter 96 Aqueous One Solution reagent. After 1-3 hours of incubation at 37° C., absorbance was measured at 490 nm in a 96-well plate reader. The background absorbance was measured in wells with only cell line medium and solution reagent. Data was calculated as a percentage of total absorbance of treated cell/absorbance of non treated cells.

Table 2 shows the functional response of selected compounds on established cell lines and primary cultures (GI₅₀, which is concentration of compound for 50% of maximal inhibition of cell proliferation); where, GI₅₀≥10 μM (+), 1 μM≤GI₅₀<10 μM (++), 100 nM≤GI₅₀<1 μM (+++) and GI₅₀<100 nM (++++). These cancer cell lines and primary cultures correspond to acute lymphocytic leukemia (ALL), CEMO-1 and to hepatocellular carcinoma cells (HCC), HepG2, Hep3B and PLC/PRF/5.

TABLE 2

| Example | CEMO-1 | HEPG2 | HEP3B | PLC/PRF/5 |
|---|---|---|---|---|
| 3-01 | ++ | ++ | ++ | ++ |
| 1-02 | + | ++ | ++ | |
| 1-03 | ++ | +++ | +++ | |
| 1-04 | +++ | +++ | +++ | |

Compounds in Table 2 inhibit proliferation of acute lymphocytic leukemia (ALL) and hepatocarcinoma (HCC) cell lines.

### REFERENCES CITED IN THE APPLICATION

Vilas-Zornoza A. et al., “Frequent and Simultaneous Epigenetic Inactivation of TP53 Pathway Genes in Acute Lymphoblastic Leukemia”, PLoS ONE 2011. 6(2): p. e17012.
Neary, R. et al, “Epigenetics and the overhealing wound: the role of DNA methylation in fibrosis”, Fibrogenesis & Tissue Repair, 2015, 8:18.
Lee S. et al., “DNA methyltransferase inhibition accelerates the immunomodulation and migration of human mesenchymal stem cells” Scientific Reports 2015, 5:8020.
Shankar S R. et al., “G9a, a multipotent regulator of gene expression”, Epigenetics, 2013. 8(1): p. 16-22.
Esteve P O. et al., “Direct interaction between DNMT1 and G9a coordinates DNA and histone methylation during replication”, Genes Dev 2006, 20:3089-3103.
Tachibana M. et al., “G9a/GLP complexes independently mediate H₃K9 and DNA methylation to silence transcription”, The EMBO Journal 2008, 27:2681-2690.
Auclair G. et al., “EHMT2 directs DNA methylation for efficient gene silencing in mouse embryos”, Genome Research 2015
Wozniak R J. et al., “5-Aza-2′-deoxycytidine-mediated reductions in G9A histone methyltransferase and histone H3 K9 di-methylation levels are linked to tumor suppressor gene reactivation”, Oncogene 2007, 26, 77-90.
Sharma S. et al., “Lysine methyltransferase G9a is not required for DNMT3A/3B anchoring to methylated nucleosomes and maintenance of DNA methylation in somatic cells”, Epigenetics Chromatin 2012, 5, 3.
Srimongkolpithak N., et al.: “Identification of 2,4-diamino-6,7-dimethoxy-quinoline derivatives as G9a inhibitors”, Med. Chem. Commun. 2014, 5, 1821-1828.

1. A compound of formula (I), or a pharmaceutically or veterinary acceptable salt thereof, or any stereoisomer or mixtures of stereoisomers, either of the compound of formula (I) or of any of its pharmaceutically or veterinary acceptable salts 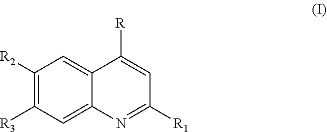
wherein
R is a radical selected from the group consisting of formula (A), formula (B), formula (C), formula (D), and formula (E): 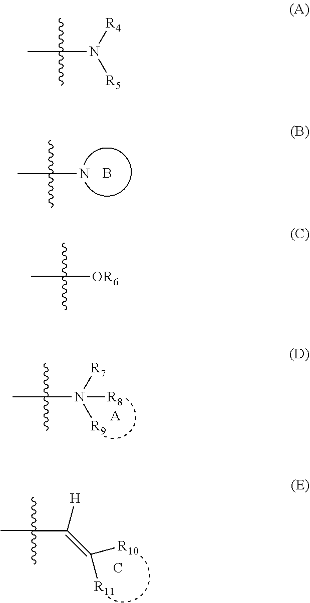
R₁ is a known ring system attached to the quinoline ring through a carbon atom, which is selected from the group consisting of:
(i) phenyl;
(ii) 5- or 6-membered heteroaromatic ring;
(iii) 3- to 7-membered saturated or partially unsaturated carbocyclic or heterocyclic monocyclic ring;
(iv) 3- to 7-membered saturated or partially unsaturated or aromatic carbocyclic or heterocyclic monocyclic ring, which is fused, bridged-fused or spiro-fused to a 3- to 7-membered saturated or partially unsaturated or aromatic carbocyclic or heterocyclic monocyclic ring;
(v) phenyl fused to a 6- to 14-membered saturated or partially unsaturated carbocyclic or heterocyclic bicyclic ring, wherein the rings of the bicyclic ring are spiro-fused; and
(vi) 5- to 6-membered heteroaromatic ring fused to a 6- to 14-membered saturated or partially unsaturated carbocyclic or heterocyclic bicyclic ring, wherein the rings of the bicyclic ring are spiro-fused;
wherein R₁ is optionally substituted with:
a) one Cy¹ or one Cy², and/or
b) one or more substituents R^{a}, and/or
c) one or more substituents Z¹ optionally substituted with one or more substituents R^{a} and/or one Cy¹;
wherein Cy¹ or Cy² are optionally substituted with one or more substituents independently selected from R^{a}, and Z² optionally substituted with one or more substituents R^{a};
R₂ is selected from the group consisting of R^{b}, halogen, —NO₂, —CN, —OR^{b′}, —OC(O)R^{b′}, —OC(O)OR^{b′}, —OC(O)NR^{b′}R^{b′}, —NR^{b′}R^{b′}, —NR^{b′}C(O)R^{b′}, —NR^{b′}C(O)OR^{b′}, —NR^{b′}C(O)NR^{b′}R^{b′}, —NR^{b′}S(O)₂R^{b′}, —NR^{b′}SO₂NR^{b′}R^{b′}R, —SR^{b′}, —S(O)R^{b′}, —S(O)OR^{b′}, —SO₂R^{b′}, —SO₂(OR^{b′}), —SO₂NR^{b′}R^{b′}, —SC(O)NR^{b′}R^{b′}, —C(O)R^{b′}, —C(O)OR^{b′}, —C(O)NR^{b′}R^{b′}, —C(O)NR^{b′}OR^{b′}, and —C(O)NR^{b′}SO₂R^{b′};
R₃ is selected from the group consisting of —OR^{d} and —OR^{e};
R₄ and R₆ are independently selected from the group consisting of Cy¹, and Z¹ optionally substituted with one or more substituents R^{a} and/or one Cy³;
wherein Cy¹ is optionally substituted with:
a) one Cy²; and/or
b) one or more substituents R^{a}, and/or
c) one or more substituents Z² optionally substituted with one or more substituents R^{a} and/or one Cy²;
wherein Cy² and Cy³ are optionally substituted with one or more substituents independently selected from R^{a}, and Z³ optionally substituted with one or more substituents R^{a};
R₅ is (C₁-C₆)alkyl optionally substituted with one or more halogen atoms or a 3- to 7-membered saturated or partially unsaturated carbocyclic monocyclic ring optionally substituted with one or more halogen atoms;
the dotted line means the presence or absence of a ring system A or C;
R₇ is absent or is selected from the group consisting of H, R^{a}, Cy¹, and Z¹ optionally substituted with one or more substituents R^{a} and/or one Cy³;
wherein Cy¹ is optionally substituted with:
a) one Cy²; and/or
b) one or more substituents R^{a}, and/or
c) one or more substituents Z² optionally substituted with one or more substituents R^{a} and/or one Cy²;
wherein Cy² and Cy³ are optionally substituted with one or more substituents independently selected from R^{a}, and Z³ optionally substituted with one or more substituents R^{a};
R₈ and R₉ are independently selected from the group consisting of H, halogen, (C₁-C₆)alkyl optionally substituted with one or more halogen atoms, and a 3- to 7-membered saturated or partially unsaturated carbocyclic monocyclic ring optionally substituted with one or more halogen atoms; or alternatively
R₈ and R₉, together with the carbon atom to which they are attached, form a known ring system A comprising a 3- to 7-membered saturated or partially unsaturated carbocyclic or heterocyclic monocyclic ring, which is optionally fused, bridged-fused or spiro-fused to a 3- to 7-membered saturated or partially unsaturated or aromatic carbocyclic or heterocyclic monocyclic ring; wherein the ring system A is optionally substituted with:
a) one Cy¹; and/or
b) one or more substituents R^{a}, and/or
c) one or more substituents Z¹ optionally substituted with one or more substituents R^{a} and/or one Cy³;
wherein Cy¹ and Cy³ are optionally substituted with one or more substituents independently selected from R^{a}, and Z² optionally substituted with one or more substituents R^{a};
R₁₀ and R₁₁ are independently selected from the group consisting of H, and Z¹ optionally substituted with one or more substituents R^{a} and/or one Cy³; wherein Cy³ are optionally substituted with one or more substituents independently selected from R^{a}, and Z³ optionally substituted with one or more substituents R^{a}; or alternatively
R₁₀ and R₁₁, together with the carbon atom to which they are attached, form a known ring system C comprising a 3- to 7-membered saturated or partially unsaturated carbocyclic or heterocyclic monocyclic ring, which is optionally fused, bridged-fused or spiro-fused to a 3- to 7-membered saturated or partially unsaturated or aromatic carbocyclic or heterocyclic monocyclic ring; wherein the ring system C is optionally substituted with:
a) one Cy¹; and/or
b) one or more substituents R^{a}, and/or
c) one or more substituents Z¹ optionally substituted with one or more substituents R^{a} and/or one Cy³;
wherein Cy¹ and Cy³ are optionally substituted with one or more substituents independently selected from R^{a}, and Z² optionally substituted with one or more substituents R^{a};
B is a known ring system comprising a 3- to 7-membered saturated or partially unsaturated heterocyclic monocyclic ring, which is optionally fused, bridged-fused or spiro-fused to a 3- to 7-membered saturated or partially unsaturated or aromatic carbocyclic or heterocyclic monocyclic ring; wherein the ring system B is optionally substituted with:
a) one Cy¹; and/or
b) one or more substituents R^{a}, and/or
c) one or more substituents Z¹ optionally substituted with one or more substituents R^{a} and/or one Cy³;
wherein Cy¹ and Cy³ are optionally substituted with one or more substituents independently selected from R^{a}, and Z² optionally substituted with one or more substituents R^{a};
each R^{a} is independently selected from the group consisting of halogen, —NO₂, —CN, —OR^{b′}, —OC(Y)R^{b′}, —OC(Y)OR^{b′}, —OC(Y)NR^{b′}R^{b′}, —NR^{b′}R^{b′}, —NR^{b′}C(Y)R^{b′}, —NR^{b′}C(Y)OR^{b′}, —NR^{b′}C(Y)NR^{b′}R^{b′}, —NR^{b′}S(O)₂R^{b′}, —NR^{b′}SO₂NR^{b′}R^{b′}, —SR^{b′}, —S(O)R^{b′}, —S(O)OR^{b′}, —SO₂R^{b′}, —SO₂(OR^{b′}), —SO₂NR^{b′}R^{b′}, —SC(Y)NR^{b′}R^{b′}, —C(Y)R^{b′}, —C(Y)OR^{b′}, —C(Y)NR^{b′}R^{b′}, —C(Y)NR^{b′}OR^{b′}, and —C(O)NR^{b′}SO₂R^{b′};
each R^{b′} is independently H or R^{b};
each R^{b} is independently selected from the group consisting of (C₁-C₆)alkyl, (C₂-C₆)alkenyl, (C₂-C₆)alkynyl, (C₂-C₆)hydrocarbon chain having one or more double bonds and one or more triple bonds, and 3- to 7-membered saturated or partially unsaturated or aromatic carbocyclic or heterocyclic monocyclic ring, wherein each R^{b} is optionally substituted with one or more halogen atoms,
R^{d} is Cy¹ optionally substituted with:
a) one Cy²; and/or
b) one or more substituents R^{a}, and/or
c) one or more substituents Z¹ optionally substituted with one or more substituents R^{a} and/or one Cy²;
wherein Cy² is optionally substituted with one or more substituents independently selected from R^{a}, and Z² optionally substituted with one or more substituents R^{a};
R^{e} is a moiety comprising at least 4 carbon atoms which is selected from the group consisting of (C₁-C₁₂)alkyl, (C₂-C₁₂)alkenyl, (C₂-C₁₂)alkynyl, and (C₂-C₆)hydrocarbon chain having one or more double bonds and one or more triple bonds; wherein R^{e} is optionally substituted with one or more substituents R^{a} and/or one Cy³; wherein Cy³ is optionally substituted with:
a) one Cy⁴; and/or
b) one or more substituents R^{a}, and/or
c) one or more substituents Z³ optionally substituted with one or more substituents R^{a} and/or one Cy⁴;
wherein Cy⁴ is optionally substituted with one or more substituents independently selected from R^{a}, and Z⁴ optionally substituted with one or more substituents R^{a};
Y is O, S, or NR^{b′};
Z¹, Z², Z³ and Z⁴ are independently selected from the group consisting of (C₁-C₁₂)alkyl, (C₂-C₁₂)alkenyl, (C₂-C₁₂)alkynyl, and (C₂-C₆)hydrocarbon chain having one or more double bonds and one or more triple bonds;
Cy¹ and Cy³ are independently a known ring system selected from the group consisting of phenyl; 5- or 6-membered heteroaromatic ring; 3- to 7-membered saturated or partially unsaturated carbocyclic or heterocyclic monocyclic ring; and 3- to 7-membered saturated or partially unsaturated or aromatic carbocyclic or heterocyclic monocyclic ring, which is fused, bridged-fused or spiro-fused to a 3- to 7-membered saturated or partially unsaturated or aromatic carbocyclic or heterocyclic monocyclic ring;
Cy², Cy⁴ are independently a known ring system selected from the group consisting of phenyl; 3- to 7-membered saturated or partially unsaturated carbocyclic or heterocyclic monocyclic ring; and 5- or 6-membered heteroaromatic ring;
wherein in the carbocyclic rings all ring members are carbon atoms; and in the heterocyclic and heteroaromatic rings one or more ring members are selected from N, O, and S; and wherein in all saturated or partially unsaturated rings one or two members of the rings are optionally C(O) and/or C(NH) and/or C[N(C₁-C₄)alkyl].

2. The compound of formula (I) according to claim 1, wherein R^{e} is a moiety comprising at least 5 carbon atoms.

3. The compound of formula (I) according to claim 1, wherein R is a radical selected from the group consisting of formula (A) and formula (B).

4. The compound of formula (I) according to claim 1, wherein R is a radical selected from the group consisting of formula (A) and formula (B), wherein:
i) when R is a radical of formula (A),
R₄ is selected from the group consisting of:
a) Cy¹ optionally substituted with one or more substituents Z², and
b) Z¹ substituted with Cy³,
wherein Z² and Cy³ in R₄ are optionally substituted as defined in claim 1; and
R₅ is (C₁-C₆)alkyl optionally substituted with one or more halogen atoms; and
ii) when R is a radical of formula (B), ring B is a known ring system comprising a 3- to 7-membered saturated or partially unsaturated heterocyclic monocyclic ring optionally substituted with:
a) one or more substituents R^{a}, and/or
b) one or more substituents Z¹;
wherein Z¹ in ring B is optionally substituted as defined in claim 1.

5. The compound of formula (I) according to claim 1, wherein R₁ is a known ring system selected from the group consisting of:
(i) phenyl;
(ii) 5- or 6-membered heteroaromatic ring;
(iii) 3- to 7-membered saturated or partially unsaturated carbocyclic or heterocyclic monocyclic ring; and
(iv) 3- to 7-membered saturated or partially unsaturated or aromatic carbocyclic or heterocyclic monocyclic ring, which is fused, bridged-fused or spiro-fused to a 3- to 7-membered saturated or partially unsaturated or aromatic carbocyclic or heterocyclic monocyclic ring;
wherein R₁ is optionally substituted as defined in claim 1.

6. The compound of formula (I) according to claim 1, wherein R₁ is a known ring system selected from the group consisting of:
(ii) 5- or 6-membered heteroaromatic ring;
(iii) 3- to 7-membered saturated or partially unsaturated heterocyclic monocyclic ring;
(iv) 5- to 6-membered aromatic carbocyclic or heterocyclic monocyclic ring, which is fused to a 5- to 6-membered aromatic carbocyclic or heterocyclic monocyclic ring;
wherein R₁ is optionally substituted as defined in claim 1.

7. The compound of formula (I) according to claim 1, wherein R₂ is selected from halogen, —CN and —OR^{b′}.

8. The compound of formula (I) according to claim 1, wherein in R₃ R^{d} and R^{e} contain at least one N atom.

9. The compound of formula (I) according to claim 1, wherein R₃ is a moiety of formula (XIV): 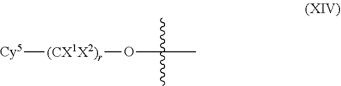
wherein
Cy⁵ is a 3- to 7-membered saturated or partially unsaturated or aromatic carbocyclic or heterocyclic monocyclic ring or a 3- to 7-membered saturated or partially unsaturated or aromatic carbocyclic or heterocyclic monocyclic ring, which is fused, bridged-fused or spiro-fused to a 3- to 7-membered saturated or partially unsaturated or aromatic carbocyclic or heterocyclic monocyclic ring, and Cy₅ is optionally substituted with one or more substituents selected from halogen and (C₁-C₃)alkyl optionally substituted with one or more halogen atoms,
X¹ and X² are independently H or halogen, and
r is a value selected from 0 to 6.

10. The compound of formula (I) according to claim 1, wherein R is a radical of formula (C), and R₆ is selected from the group consisting of:
a) Cy¹ optionally substituted with one or more substituents Z², and
b) Z¹ substituted with Cy³;
wherein Z² and Cy³ in R₆ are optionally substituted as defined in claim 1.

11. The compound of formula (I) according to claim 1, wherein R is a radical of formula (D), and
i) the dotted line means the absence of a ring system A;
R₇ is selected from the group consisting of:
a) Cy¹ optionally substituted with one or more substituents Z², and
b) Z¹ substituted with Cy³;
wherein Z² and Cy³ in R₇ are optionally substituted as defined in claim 1; and
R₈ and R₉ are independently selected from the group consisting of H, halogen, (C₁-C₆)alkyl optionally substituted with one or more halogen atoms, and a 3- to 7-membered saturated or partially unsaturated carbocyclic monocyclic ring optionally substituted with one or more halogen atoms; or alternatively
ii) the dotted line means the presence of a ring system A;
R₇ is selected from the group consisting of:
a) Cy¹ optionally substituted with one or more substituents Z², and
b) Z¹ substituted with Cy³;
wherein Z² and Cy³ in R₇ are optionally substituted as defined in claim 1; and
R₈ and R₉, together with the carbon atom to which they are attached form a known ring system A comprising a 3- to 7-membered saturated or partially unsaturated carbocyclic monocyclic ring optionally substituted as defined in claim 1; or alternatively
iii) the dotted line means the presence of a ring system A;
R₇ is selected from the group consisting of H, R^{a}, and Z¹ optionally substituted with one or more substituents R^{a}; and
R₈ and R₉, together with the carbon atom to which they are attached form a known ring system A comprising a 3- to 7-membered saturated or partially unsaturated carbocyclic or heterocyclic monocyclic ring optionally substituted with one or more substituents Z¹, wherein Z¹ in ring A is optionally substituted as defined in claim 1.

12. The compound of formula (I) according to claim 1, wherein R is a radical of formula (E), and R₁₀ and R₁₁, together with the carbon atom to which they are attached, form a known ring system C comprising a 3- to 7-membered saturated or partially unsaturated carbocyclic or heterocyclic monocyclic ring, wherein the ring system C is optionally substituted as defined in claim 1.

13. A pharmaceutical or veterinary composition which comprises a therapeutically effective amount of a compound of formula (I) as defined in claim 1, or a pharmaceutically or veterinary acceptable salt thereof, or any stereoisomer or mixtures of stereoisomers, either of the compound of formula (I) or of its pharmaceutically or veterinary acceptable salt, together with one or more pharmaceutically or veterinary acceptable excipients or carriers.

14. A method for the treatment and/or prevention of cancer, fibrosis and/or immunomodulation, comprising administering an effective amount of a compound of formula (I) as defined in claim 1, and one or more pharmaceutically or veterinary acceptable excipients or carriers, in a subject in need thereof, including a human.

15. The method according to claim 14, wherein the cancer is selected from the group consisting of Acute Lymphocytic Leukemia (ALL), Diffuse Large B-cell lymphoma (DLBCL), bladder cancer, breast cancer, cervical cancer, colorectal cancer, glioblastoma, hepatocarcinoma, melanoma, pancreatic cancer, prostate cancer, renal cancer, small-cell lung cancer, non small-cell lung cancer, acute myeloid leukemia, mantle cell lymphoma and multiple myeloma.

16. The compound of formula (I) according to claim 1, wherein R₁ is a known ring system selected from the group consisting of:
(i) phenyl;
(ii) 5- or 6-membered heteroaromatic ring;
(iii) 3- to 7-membered saturated or partially unsaturated carbocyclic or heterocyclic monocyclic ring; and
(iv) 3- to 7-membered saturated or partially unsaturated or aromatic carbocyclic or heterocyclic monocyclic ring, which is fused, bridged-fused or spiro-fused to a 3- to 7-membered saturated or partially unsaturated or aromatic carbocyclic or heterocyclic monocyclic ring;
wherein R₁ is optionally substituted as defined in claim 1; R₂ is selected from halogen, —CN and —OR^{b′}; and in R₃ R^{d} and R^{e} contain at least one N atom.

17. The compound of formula (I) according to claim 1, wherein R₁ is a known ring system selected from the group consisting of:
(i) phenyl;
(ii) 5- or 6-membered heteroaromatic ring;
(iii) 3- to 7-membered saturated or partially unsaturated carbocyclic or heterocyclic monocyclic ring; and
(iv) 3- to 7-membered saturated or partially unsaturated or aromatic carbocyclic or heterocyclic monocyclic ring, which is fused, bridged-fused or spiro-fused to a 3- to 7-membered saturated or partially unsaturated or aromatic carbocyclic or heterocyclic monocyclic ring;
wherein R₁ is optionally substituted as defined in claim 1; R₂ is selected from halogen, —CN and —OR^{b′}; and R₃ is a moiety of formula (XIV): 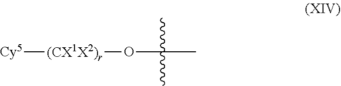
wherein
Cy⁵ is a 3- to 7-membered saturated or partially unsaturated or aromatic carbocyclic or heterocyclic monocyclic ring or a 3- to 7-membered saturated or partially unsaturated or aromatic carbocyclic or heterocyclic monocyclic ring, which is fused, bridged-fused or spiro-fused to a 3- to 7-membered saturated or partially unsaturated or aromatic carbocyclic or heterocyclic monocyclic ring, and Cy₅ is optionally substituted with one or more substituents selected from halogen and (C₁-C₃)alkyl optionally substituted with one or more halogen atoms,
X¹ and X² are independently H or halogen, and
r is a value selected from 0 to 6.

18. The compound of formula (I) according to claim 2, wherein R is a radical selected from the group consisting of formula (A) and formula (B).

19. The compound of formula (I) according to claim 1, wherein R^{e} is a moiety comprising at least 5 carbon atoms, wherein R is a radical selected from the group consisting of formula (A) and formula (B), and wherein R₁ is a known ring system selected from the group consisting of:
(i) phenyl;
(ii) 5- or 6-membered heteroaromatic ring;
(iii) 3- to 7-membered saturated or partially unsaturated carbocyclic or heterocyclic monocyclic ring; and
(iv) 3- to 7-membered saturated or partially unsaturated or aromatic carbocyclic or heterocyclic monocyclic ring, which is fused, bridged-fused or spiro-fused to a 3- to 7-membered saturated or partially unsaturated or aromatic carbocyclic or heterocyclic monocyclic ring;
wherein R₁ is optionally substituted as defined in claim 1; R₂ is selected from halogen, —CN and —OR^{b′}; and in R₃ R^{d} and R^{e} contain at least one N atom.

20. The compound of formula (I) according to claim 1, wherein R^{e} is a moiety comprising at least 5 carbon atoms, wherein R is a radical selected from the group consisting of formula (A) and formula (B), and wherein R₁ is a known ring system selected from the group consisting of:
(i) phenyl;
(ii) 5- or 6-membered heteroaromatic ring;
(iii) 3- to 7-membered saturated or partially unsaturated carbocyclic or heterocyclic monocyclic ring; and
(iv) 3- to 7-membered saturated or partially unsaturated or aromatic carbocyclic or heterocyclic monocyclic ring, which is fused, bridged-fused or spiro-fused to a 3- to 7-membered saturated or partially unsaturated or aromatic carbocyclic or heterocyclic monocyclic ring;
wherein R₁ is optionally substituted as defined in claim 1; R₂ is selected from halogen, —CN and —OR^{b′}; and R₃ is a moiety of formula (XIV): 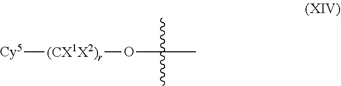
wherein
Cy⁵ is a 3- to 7-membered saturated or partially unsaturated or aromatic carbocyclic or heterocyclic monocyclic ring or a 3- to 7-membered saturated or partially unsaturated or aromatic carbocyclic or heterocyclic monocyclic ring, which is fused, bridged-fused or spiro-fused to a 3- to 7-membered saturated or partially unsaturated or aromatic carbocyclic or heterocyclic monocyclic ring, and Cy₅ is optionally substituted with one or more substituents selected from halogen and (C₁-C₃)alkyl optionally substituted with one or more halogen atoms,
X¹ and X² are independently H or halogen, and
r is a value selected from 0 to 6.
